(19) United States
(12) Patent Application Publication
(10) Pub. No.: US 2024/0277915 A1
(43) Pub. Date: Aug. 22, 2024

(54) CLOSED-LOOP ADIPOSE TRANSPLANT SYSTEMS AND KITS, CONTROLLERS, AND METHODS RELATED THERETO

(71) Applicant: THE METHODIST HOSPITAL SYSTEM, Houston, TX (US)

(72) Inventors: Aldona J. SPIEGEL, Houston, TX (US); Andres Felipe DOVAL, Houston, TX (US); Robert BENKOWSKI, Fort Worth, TX (US); Antonio Armando ARAUJO, Fort Worth, TX (US)

(21) Appl. No.: 18/569,862

(22) PCT Filed: Jun. 16, 2022
(86) PCT No.: PCT/US2022/033824

§ 371 Date: Dec. 13, 2023

Related U.S. Application Data
(60) Provisional application No. 63/211,043, filed on Jun. 16, 2021.

(51) Int. Cl. A61M 1/00

(52) U.S. Cl. CPC A61M 1/892

(57) ABSTRACT

Disclosed herein are kits of consumable parts for a closed-loop adipose transplant system, closed-loop adipose transplant systems, controllers therefor, and methods of use thereof.

### CROSS-REFERENCE TO RELATED APPLICATIONS

This application claims the benefit of priority to U.S. Provisional Application No. 63/211,043 filed Jun. 16, 2021, which is hereby incorporated herein by reference in its entirety.

### BACKGROUND

Autologous fat grafting is a surgical procedure that involves harvesting, processing, and transferring adipose tissue from one anatomical region of the patient to another.

Fat grafting is mainly used for the treatment of volume and contour abnormalities and congenital breast deformities. It is a technically demanding and time-consuming procedure involving several steps to harvest, process, and transfer fat.

Currently there are few devices to facilitate fat graft transfer, all have significant limitations. These limitations include intraoperative inefficiency, inability to graft and process at the same time, ergonomics, manual injection, and quality of fat harvested.

The devices, methods, and systems discussed herein addresses these and other needs.

### SUMMARY

In accordance with the purposes of the disclosed devices, methods, and systems as embodied and broadly described herein, the disclosed subject matter relates to kits of consumable parts for a closed-loop adipose transplant system, closed-loop adipose transplant systems, controllers therefor, and methods of use thereof.

Additional advantages of the disclosed devices, systems, and methods will be set forth in part in the description which follows, and in part will be obvious from the description. The advantages of the disclosed devices, systems, and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1 is a schematic view of an example closed-loop adipose transplant system as disclosed herein according to one implementation.

FIG. 2 is a schematic view of an example closed-loop adipose transplant system as disclosed herein according to one implementation.

FIG. 3 is a schematic view of an example adipose separation module 200 as disclosed herein according to one implementation.

FIG. 4 is a schematic view of an example adipose separation module 200 as disclosed herein according to one implementation.

FIG. 5 is a schematic view of an example adipose separation module 200 as disclosed herein according to one implementation.

FIG. 6 is a schematic view of an example rotary implement 230 as disclosed herein according to one implementation.

FIG. 7 is a schematic view of an example rotary implement 230 as disclosed herein according to one implementation.

FIG. 8 is a schematic view of an example rotary implement 230 as disclosed herein according to one implementation.

FIG. 9 is a schematic view of an example rotary implement 230 as disclosed herein according to one implementation.

FIG. 10 is a schematic view of an example rotary implement 230 as disclosed herein according to one implementation.

FIG. 11 is a schematic view of an example adipose separation module 200 as disclosed herein according to one implementation.

FIG. 12 is a schematic view of an example adipose separation module 200 disposed on a platform 226 as disclosed herein according to one implementation.

FIG. 13 is a schematic view of an example adipose separation module 200 as disclosed herein according to one implementation.

FIG. 14 is a schematic view of an example adipose separation module 200 as disclosed herein according to one implementation.

FIG. 15 is a schematic view of an example closed-loop adipose transplant system as disclosed herein according to one implementation.

FIG. 16 is a schematic view of an example closed-loop adipose transplant system as disclosed herein according to one implementation.

FIG. 17 is a schematic view of an example effluent separation module 300 as disclosed herein according to one implementation.

FIG. 18 is a schematic view of an example effluent separation module 300 as disclosed herein according to one implementation.

FIG. 19 is a schematic view of an example effluent separation module 300 as disclosed herein according to one implementation.

FIG. 20 is a schematic view of an example rotary implement 330 as disclosed herein according to one implementation.

FIG. 21 is a schematic view of an example rotary implement 330 as disclosed herein according to one implementation.

FIG. 22 is a schematic view of an example rotary implement 330 as disclosed herein according to one implementation.

FIG. 23 is a schematic view of an example rotary implement 330 as disclosed herein according to one implementation.

FIG. 24 is a schematic view of an example rotary implement 330 as disclosed herein according to one implementation.

FIG. 25 is a schematic view of an example effluent separation module 300 as disclosed herein according to one implementation.

FIG. 26 is a schematic view of an example effluent separation module 300 as disclosed herein according to one implementation.

FIG. 27 is a schematic view of an example effluent separation module 300 as disclosed herein according to one implementation.

FIG. 28 is a schematic view of an example effluent separation module 300 as disclosed herein according to one implementation.

FIG. 29 is a schematic view of an example closed-loop adipose transplant system as disclosed herein according to one implementation.

FIG. 30 is a schematic view of an example closed-loop adipose transplant system as disclosed herein according to one implementation.

FIG. 31 is a schematic view of an example closed-loop adipose transplant system as disclosed herein according to one implementation.

FIG. 32 is a schematic view of an example closed-loop adipose transplant system as disclosed herein according to one implementation.

FIG. 33 is a schematic view of an example closed-loop adipose transplant system as disclosed herein according to one implementation.

FIG. 34 is a schematic view of an example closed-loop adipose transplant system as disclosed herein according to one implementation.

FIG. 35 is a schematic view of an example ergonomic injection handle 400 as disclosed herein according to one implementation.

FIG. 36 is an example of a user interface as disclosed herein according to one implementation.

FIG. 37 is an example of a user interface as disclosed herein according to one implementation.

FIG. 38 is a schematic illustrating of an example computing device.

FIG. 39 is an example logic flow chart.

FIG. 40 is a schematic diagram of an example system as disclosed herein according to one implementation.

FIG. 41 is a schematic diagram of an example system as disclosed herein according to one implementation.

FIG. 42 is an image of an example roller pump.

FIG. 43 is a schematic partial cross-sectional plan view showing the tip clearance.

FIG. 44 is a schematic partial cross-sectional plan view showing the tip clearance.

FIG. 45 is a schematic diagram of an example fat filtration module 200 including a sliding ring 224 as disclosed herein according to one implementation.

FIG. 46 is a schematic cross-sectional plan view of an example fat filtration module 200 including a sliding ring 224 as disclosed herein according to one implementation.

FIG. 47 is a schematic diagram of an example filter cage 222 as disclosed herein according to one implementation.

FIG. 48 is and exploded view of an example fat filtration module 200 including an auger 230 and filter cage 222 as disclosed herein according to one implementation.

FIG. 49 is an image of an example pressure sensor.

FIG. 50 is an example block diagram for motor/pump control.

FIG. 51 is a schematic cross-sectional plan view of an adipose filtration module 200 including a gear pump as disclosed herein according to one implementation.

FIG. 52 is a schematic cross-sectional plan view of an adipose filtration module 200 including the rotary implement 230 of FIG. 10 as disclosed herein according to one implementation.

FIG. 53 a schematic cross-sectional view of an adipose filtration module 200 including the rotary implement 230 of FIG. 10 as disclosed herein according to one implementation.

FIG. 54 a schematic cross-sectional view of an adipose filtration module 200 including the rotary implement 230 of FIG. 7 as disclosed herein according to one implementation.

FIG. 55 is a schematic diagram of an example system as disclosed herein according to one implementation.

FIG. 56 is a schematic diagram of an example auger-motor assembly as disclosed herein according to one implementation.

FIG. 57 is a schematic diagram of an example auger-motor assembly with a filter cage as disclosed herein according to one implementation.

FIG. 58 is a schematic cross-sectional plan view showing the clearance between the auger and the cage.

FIG. 59 is an image of an example system as disclosed herein according to one implementation.

FIG. 60 is a schematic diagram of an injector 908 as disclosed herein according to one implementation.

FIG. 61 is a schematic diagram of an example system as disclosed herein according to one implementation.

### DETAILED DESCRIPTION

The devices, methods, and systems described herein may be understood more readily by reference to the following detailed description of specific aspects of the disclosed subject matter and the Examples included therein.

Before the present devices, methods, and systems are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included. As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

### Kits and Systems

Disclosed herein are kits of consumable parts for a closed-loop adipose transplant system (e.g., a closed-loop autologous fat grafting system).

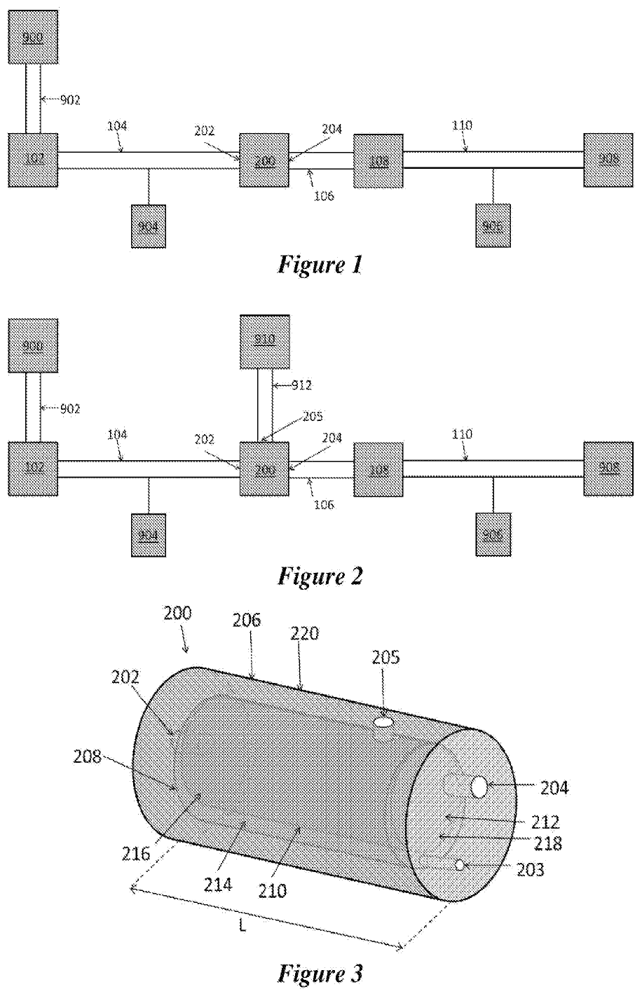

Referring now to FIG. 1, disclosed herein are kits 100 of consumable parts for a closed-loop adipose transplant system including a liposuction cannula 900, a first tube 902, a first pump 904, a second pump 906, and an injector 908, the kit 100 comprising: a collection canister 102; a second tube 104; an adipose separation module 200 having an inlet 202 and an outlet 204; a third tube 106; a collection reservoir 108; and a fourth tube 110; wherein the consumable parts of the kit 100 together form a continuous, closed fluid pathway for the adipose tissue from the liposuction cannula 900 to the collection canister 102 through the first tube 902, from the collection canister 102 to the inlet 202 of the adipose separation module 200 through the second tube 104, from the inlet 202 to the outlet 204 through the adipose separation module 200, from the outlet 204 of the adipose separation module 200 to the collection reservoir 108 through the third tube 106, and from the collection reservoir 108 to the injector 908 through the fourth tube 110.

The collection canister 102 is configured to receive and collect a mixture comprising adipose tissue harvested from a first anatomical region by the liposuction cannula 900 via the first tube 902.

The collection canister 102 can, for example, comprise any suitable container for collecting adipose tissue, such as those known in the art. In some examples, the collection canister 102 can comprise a suction canister or a vacutainer, such as a liposuction vacutainer. The collection canister 102 can, for example, comprise any suitable material, such as those known in the art. For example, the collection canister 102 can comprise glass, a polymer, or a combination thereof. For example, the collection canister 102 can comprise a polymer liner disposed inside of a rigid canister.

The collection canister 102 is a closed container configured to be fluidly coupled to the first tube 902 and the second tube 104. For example, the collection canister 102 can have an interior volume defined by a wall, wherein the wall has one or more ports independently configured to receive the first tube 902 and/or the second tube 104, such that the interior volume of the collection canister 102 is fluidly connected to the first tube 902 and the second tube 104. In some examples, the collection canister 102 can comprise a body and a lid, wherein the lid is configured to be coupled to the body (e.g., removably coupled), such that when the lid and the body are coupled they define the interior volume. The lid can, for example, have a first port and a second port, the first port being configured to receive the first tube 904 and the second port being configured to receive the second tube 104.

The second tube 104 is configured to fluidly connect the collection canister 102 to the inlet 202 of the adipose separation module 200. The second tube 104 is further configured to fluidly connect the first pump 904 to the collection canister 102 and the adipose separation module 200, such that the second tube 104 communicates a pressure applied by the first pump 904, the pressure being sufficient to: transport the mixture through the second tube 104 from the collection canister 102 to the inlet 202 of the adipose separation module 200. In some examples, the pressure applied by the first pump 904 is sufficient to overcome the liposuction pressure in the collection canister 102.

The adipose separation module 200 is configured to: receive the mixture from the collection canister 102 through the inlet 202 and separate the adipose tissue from the mixture, thereby concentrating the adipose tissue from the mixture.

The third tube 106 is configured to fluidly connect the outlet 204 of the adipose separation module 200 to the collection reservoir 108.

The collection reservoir 108 is configured to receive and collect the concentrated adipose tissue from outlet 204 of the adipose separation module 200 via the third tube 106.

The collection reservoir 108 can, for example, comprise any suitable container for collecting adipose tissue, such as those known in the art. In some examples, the collection reservoir 108 can comprise a sterile medical collection bag.

The collection reservoir 108 can, for example, comprise any suitable material, such as those known in the art. For example, the collection reservoir 108 can comprise glass, a polymer, or a combination thereof. The collection reservoir 108 can, for example, be rigid or flexible. In some examples, the collection reservoir 108 can expand as it is filled with adipose tissue.

The collection reservoir 108 is a closed container configured to be fluidly coupled to the third tube 106 and the fourth tube 110. For example, the collection reservoir 108 can have an interior volume defined by a wall, wherein the wall has one or more ports independently configured to receive the third tube 106 and/or the fourth tube 110, such that the interior volume of the collection reservoir 108 is fluidly connected to the third tube 106 and the fourth tube 110. In some examples, the collection reservoir 108 can comprise a body and a lid, wherein the lid is configured to be coupled to the body (e.g., removably coupled), such that when the lid and the body are coupled they define the interior volume. The lid can, for example, have a first port and a second port, the first port being configured to receive the third tube 106 and the second port being configured to receive the fourth tube 110.

The fourth tube 110 is configured to fluidly connect the collection reservoir 108 to the injector 908. The fourth tube 110 is further configured to fluidly connect the second pump 906 to the collection reservoir 108 and the injector 908, such that the fourth tube 110 communicates a pressure applied by the second pump 906, the pressure being sufficient to: transport the concentrated adipose through the fourth tube 110 from the collection reservoir 108 to the injector 908 and inject the concentrated adipose tissue into a second anatomical region.

The adipose separation module 200 has an inlet 202 and an outlet 204 and is configured to: receive the mixture from the collection canister 102 through the inlet 202 and separate the adipose tissue from the mixture, thereby concentrating the adipose tissue from the mixture. For example, the adipose separation module 200 can be configured to filter the viable adipose tissue from plasma, blood remnants, lysed adipocyte cells, etc.

The inlet 202 of the adipose separation module 200 can, for example, have an inner diameter of 1 millimeter (mm) or more (e.g., 1.25 mm or more, 1.5 mm or more, 1.75 mm or more, 2 mm or more, 2.25 mm or more, 2.5 mm or more, 2.75 mm or more, 3 mm or more, 3.25 mm or more, 3.5 mm or more, 3.75 mm or more, 4 mm or more, 4.25 mm or more, 4.5 mm or more, 4.75 mm or more, 5 mm or more, 5.25 mm or more, 5.5 mm or more, 5.75 mm or more, 6 mm or more, 6.25 mm or more, 6.5 mm or more, 6.75 mm or more, 7 mm or more, 7.25 mm or more, 7.5 mm or more, 7.75 mm or more, 8 mm or more, 8.25 mm or more, 8.5 mm or more, 8.75 mm or more, 9 mm or more, 9.25 mm or more, 9.5 mm or more, or 9.75 mm or more). In some examples, the inlet 202 of the adipose separation module 200 can have an inner diameter of 10 mm or less (e.g., 9.75 mm or less, 9.5 mm or less, 9.25 mm or less, 9 mm or less, 8.75 mm or less, 8.5 mm or less, 8.25 mm or less, 8 mm or less, 7.75 mm or less, 7.5 mm or less, 7.25 mm or less, 7 mm or less, 6.75 mm or less, 6.5 mm or less, 6.25 mm or less, 6 mm or less, 5.75 mm or less, 5.5 mm or less, 5.25 mm or less, 5 mm or less, 4.75 mm or less, 4.5 mm or less, 4.25 mm or less, 4 mm or less, 3.75 mm or less, 3.5 mm or less, 3.25 mm or less, 3 mm or less, 2.75 mm or less, 2.5 mm or less, 2.25 mm or less, 2 mm or less, 1.75 mm or less, 1.5 mm or less, or 1.25 mm or less). The inner diameter of inlet 202 of the adipose separation module 200 can range from any of the minimum values described above to any of the maximum values described above. For example, the inlet 202 of the adipose separation module 200 can have an inner diameter of from 1 millimeter (mm) to 10 mm (e.g., from 1 mm to 5 mm, from 5 mm to 10 mm, from 1 mm to 2 mm, from 2 mm to 4 mm, from 4 mm to 6 mm, from 6 mm to 8 mm, from 8 mm to 10 mm, from 1 mm to 9 mm, from 2 mm to 10 mm, from 2 mm to 9 mm, from 1 mm to 6 mm, from 1 mm to 3.5 mm, from 3.5 mm to 6 mm, from 1 mm to 2 mm, from 2 mm to 3 mm, from 3 mm to 4 mm, from 4 mm to 5 mm, from 5 mm to 6 mm, from 1 mm to 5.5 mm, from 1.5 mm to 5.5 mm, from 1.5 mm to 5.5 mm, from 2 mm to 6 mm, from 3 mm to 6 mm, from 4 mm to 6 mm, or from 5.25 mm to 5.5 mm). In some examples, the inlet 202 of the adipose separation module 200 can have an inner diameter of from 5.25 mm to 5.5 mm.

The outlet 204 of the adipose separation module 200 can, for example, have an inner diameter of 1 millimeter (mm) or more (e.g., 1.25 mm or more, 1.5 mm or more, 1.75 mm or more, 2 mm or more, 2.25 mm or more, 2.5 mm or more, 2.75 mm or more, 3 mm or more, 3.25 mm or more, 3.5 mm or more, 3.75 mm or more, 4 mm or more, 4.25 mm or more, 4.5 mm or more, 4.75 mm or more, 5 mm or more, 5.25 mm or more, 5.5 mm or more, 5.75 mm or more, 6 mm or more, 6.25 mm or more, 6.5 mm or more, 6.75 mm or more, 7 mm or more, 7.25 mm or more, 7.5 mm or more, 7.75 mm or more, 8 mm or more, 8.25 mm or more, 8.5 mm or more, 8.75 mm or more, 9 mm or more, 9.25 mm or more, 9.5 mm or more, or 9.75 mm or more). In some examples, the outlet 204 of the adipose separation module 200 can have an inner diameter of 10 mm or less (e.g., 9.75 mm or less, 9.5 mm or less, 9.25 mm or less, 9 mm or less, 8.75 mm or less, 8.5 mm or less, 8.25 mm or less, 8 mm or less, 7.75 mm or less, 7.5 mm or less, 7.25 mm or less, 7 mm or less, 6.75 mm or less, 6.5 mm or less, 6.25 mm or less, 6 mm or less, 5.75 mm or less, 5.5 mm or less, 5.25 mm or less, 5 mm or less, 4.75 mm or less, 4.5 mm or less, 4.25 mm or less, 4 mm or less, 3.75 mm or less, 3.5 mm or less, 3.25 mm or less, 3 mm or less, 2.75 mm or less, 2.5 mm or less, 2.25 mm or less, 2 mm or less, 1.75 mm or less, 1.5 mm or less, or 1.25 mm or less). The inner diameter of the outlet 204 adipose separation module 200 can range from any of the minimum values described above to any of the maximum values described above. For example, the outlet 204 of the adipose separation module 200 can have an inner diameter of from 1 millimeter (mm) to 10 mm (e.g., from 1 mm to 5 mm, from 5 mm to 10 mm, from 1 mm to 2 mm, from 2 mm to 4 mm, from 4 mm to 6 mm, from 6 mm to 8 mm, from 8 mm to 10 mm, from 1 mm to 9 mm, from 2 mm to 10 mm, from 2 mm to 9 mm, from 1 mm to 6 mm, from 1 mm to 3.5 mm, from 3.5 mm to 6 mm, from 1 mm to 2 mm, from 2 mm to 3 mm, from 3 mm to 4 mm, from 4 mm to 5 mm, from 5 mm to 6 mm, from 1 mm to 5.5 mm, from 1.5 mm to 5.5 mm, from 1.5 mm to 5.5 mm, from 2 mm to 6 mm, from 3 mm to 6 mm, from 4 mm to 6 mm, or from 5.25 mm to 5.5 mm). In some examples, the outlet 204 of the adipose separation module 200 can have an inner diameter of from 5.25 mm to 5.5 mm.

In some examples, the adipose separation module 200 is further configured to contact the mixture with a wash liquid, thereby washing the mixture, and to separate the adipose tissue from the mixture and the wash liquid, thereby concentrating the adipose tissue. The wash liquid can comprise any suitable wash liquid, such as those known in the art. Examples of suitable wash liquids include, but are not limited to, saline, lactated ringer's, collagenase, pH buffers, sterile water, and combinations thereof. In some examples, the wash liquid comprises saline, lactated ringers, or a combination thereof.

Referring now to FIG. 2, the adipose separation module 200 can, in some examples, further comprise an orifice 205 and the adipose separation module 200 can be configured to receive the wash liquid through the orifice 205. The orifice 205 of the adipose separation module 200 can, for example, have an inner diameter of 1 millimeter (mm) or more (e.g., 1.25 mm or more, 1.5 mm or more, 1.75 mm or more, 2 mm or more, 2.25 mm or more, 2.5 mm or more, 2.75 mm or more, 3 mm or more, 3.25 mm or more, 3.5 mm or more, 3.75 mm or more, 4 mm or more, 4.25 mm or more, 4.5 mm or more, 4.75 mm or more, 5 mm or more, 5.25 mm or more, 5.5 mm or more, or 5.75 mm or more). In some examples, the orifice 205 of the adipose separation module 200 can have an inner diameter of 6 mm or less (e.g., 5.75 mm or less, 5.5 mm or less, 5.25 mm or less, 5 mm or less, 4.75 mm or less, 4.5 mm or less, 4.25 mm or less, 4 mm or less, 3.75 mm or less, 3.5 mm or less, 3.25 mm or less, 3 mm or less, 2.75 mm or less, 2.5 mm or less, 2.25 mm or less, 2 mm or less, 1.75 mm or less, 1.5 mm or less, or 1.25 mm or less). The inner diameter of the orifice 205 the adipose separation module 200 can range from any of the minimum values described above to any of the maximum values described above. For example, the orifice 205 of the adipose separation module 200 can have an inner diameter of from 1 millimeter (mm) to 6 mm (e.g., from 1 mm to 3.5 mm, from 3.5 mm to 6 mm, from 1 mm to 2 mm, from 2 mm to 3 mm, from 3 mm to 4 mm, from 4 mm to 5 mm, from 5 mm to 6 mm, from 1 mm to 5.5 mm, from 1.5 mm to 5.5 mm, from 1.5 mm to 5.5 mm, from 2 mm to 6 mm, from 3 mm to 6 mm, from 4 mm to 6 mm, or from 5.25 mm to 5.5 mm).

The orifice 205 can, for example, be configured to be fluidly connected to a wash liquid reservoir 910 configured to contain the wash liquid, for example via a fifth tube 912. For example, the orifice 205 can be configured to be fluidly connected to the fifth tube 912, wherein the fifth tube 912 is configured to fluidly connect the orifice 205 of the adipose separation module 200 to a wash liquid reservoir 910 configured to contain the wash liquid. In some examples, the kit 100 further comprises a fifth tube 912 configured to fluidly connect the orifice 205 of the adipose separation module 200 to a wash liquid reservoir 910 configured to contain the wash liquid. In some examples, the kit 100 can further comprise a wash liquid reservoir 910 configured to be fluidly connected to the orifice 205. In some examples, the kit 100 can further comprise the fifth tube 912 and the wash liquid reservoir 910.

The wash liquid reservoir 910 can, for example, comprise any suitable container for containing a wash liquid, such as those known in the art. The wash liquid reservoir 910 can, for example, comprise any suitable material, such as those known in the art. For example, the wash liquid reservoir 910 can comprise a polymer, glass, or a combination thereof. In some examples, the wash liquid reservoir 910 can be rigid or flexible. In some examples, the wash liquid reservoir 910 can comprise a sterile medical collection bag, a rigid polymer vessel, a glass vessel, or a combination thereof.

Referring now to FIG. 3, in some examples, the adipose separation module 200 further comprises: a housing 206 defining an interior cavity 208, the inlet 202, the outlet 204, and the orifice 205 (when present). In some examples, the adipose separation module 200 further comprises a filter 210 configured to be disposed within the interior cavity 208, such that the filter 210 is configured to define a first compartment 212 and a second compartment 214 within the interior cavity 208, the first compartment 212 being a portion of the interior cavity 208 encompassed by the filter 210 and the second compartment 214 being a portion of the interior cavity 208 outside the filter 210; wherein the first compartment 212 has a proximal end 216 and a distal end 218, the inlet 202 of the adipose separation module 200 being fluidly connected to the first compartment 212 at or near the proximal end 216 of the first compartment 212, and the outlet 204 of the adipose separation module 200 being fluidly connected to the first compartment 212 at or near the distal end 218 of the first compartment 212. In some examples, the orifice 205 of the adipose separation module (when present), can be fluidly connected to the interior cavity 208. The housing 206 can have a top surface 220, and the orifice 205 (when present) can be defined by the top surface 220 of the housing 206.

In some examples, the filter 210 is configured to separate the adipose tissue from the wash liquid (when present) and other components in the mixture by passing the wash liquid (when present) and the other components from the mixture through the filter 210 into the second compartment 214 as the mixture is transported through the housing 206, thereby concentrating the adipose tissue within the first compartment 212 and forming an effluent in the second compartment 214, the effluent comprising the wash liquid (when present) and the other components from the mixture.

The housing 206 can comprise any suitable material, such as those known in the art. Examples of suitable materials for the housing 206 include, but are not limited to, polymers, such as transparent and semi-transparent polymers. In some examples, the housing 206 can comprise polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof. In some examples, the housing 206 can comprise polycarbonate.

The housing 206 can have any shape, such as a regular shape, an irregular shape, an isotropic shape, or an anisotropic shape. For example, the housing 206 can be a polyhedron (e.g., a platonic solid, a prism, a pyramid), a cylinder, a hemicylinder, an elliptical cylinder, a hemi-elliptical cylinder, a cone, a semicone, etc. In some examples, the housing 206 can have a cylindrical shape.

The housing 206 has a longitudinal axis, a proximal end, and a distal end opposite and axially spaced apart from the proximal end. The housing 206 has a length, the length being the dimension along the longitudinal axis from the proximal end to the distal end (e.g., “L” in FIG. 3). In some examples, the housing 206 has a length of 50 mm or more (e.g., 55 mm or more, 60 mm or more, 65 mm or more, 70 mm or more, 75 mm or more, 80 mm or more, 85 mm or more, 90 mm or more, 95 mm or more, 100 mm or more, 110 mm or more, 120 mm or more, 130 mm or more, 140 mm or more, 150 mm or more, 160 mm or more, 170 mm or more, 180 mm or more, or 190 mm or more). In some example, the housing 206 has a length of 200 mm or less (e.g., 190 mm or less, 180 mm or less, 170 mm or less, 160 mm or less, 150 mm or less, 140 mm or less, 130 mm or less, 120 mm or less, 110 mm or less, 100 mm or less, 95 mm or less, 90 mm or less, 85 mm or less, 80 mm or less, 75 mm or less, 70 mm or less, 65 mm or less, 60 mm or less, or 55 mm or less). The length of the housing 206 can range from any of the minimum values described above to any of the maximum values described above. For example, the housing 206 can have a length of from 50 mm to 200 mm (e.g., from 50 mm to 125 mm, from 125 mm to 200 mm, from 50 mm to 100 mm, from 100 mm to 150 mm, from 150 mm to 200 mm, from 50 mm to 175 mm, from 75 mm to 200 mm, from 75 mm to 174 mm, from 50 mm to 150 mm, from 60 mm to 90 mm, from 70 mm to 80 mm, or from 75 mm to 80 mm). In some examples, the housing 206 can have a length of from 75 mm to 80 mm.

The housing 206 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the housing 206 can be substantially circular, ovate, ovoid, elliptic, triangular, rectangular, polygonal, etc.

The housing 206 can have an average characteristic dimension. The term “characteristic dimension,” as used herein refers to the largest straight line distance between two points in the plane of the cross-sectional shape of the housing 206. “Average characteristic dimension” and “mean characteristic dimension” are used interchangeably herein, and generally refer to the statistical mean characteristic dimension. For example, for a cylindrical housing 206, the cross-sectional shape can be substantially circular and the average characteristic dimension can refer to the average diameter.

For example, the housing 206 can have an average characteristic dimension of 15 mm or more (e.g., 16 mm or more, 17 mm or more, 18 mm or more, 19 mm or more, 20 mm or more, 21 mm or more, 22 mm or more, 23 mm or more, 24 mm or more, 25 mm or more, 26 mm or more, 27 mm or more, 28 mm or more, 29 mm or more, 30 mm or more, 31 mm or more, 32 mm or more, 33 mm or more, 34 mm or more, 35 mm or more, 36 mm or more, 37 mm or more, 38 mm or more, 39 mm or more, 40 mm or more, 45 mm or more, 50 mm or more, 55 mm or more, 60 mm or more, 65 mm or more, or 70 mm or more). In some examples, the housing 206 can have an average characteristic dimension of 75 mm or less (e.g., 70 mm or less, 65 mm or less, 60 mm or less, 55 mm or less, 50 mm or less, 45 mm or less, 40 mm or less, 39 mm or less, 38 mm or less, 37 mm or less, 36 mm or less, 35 mm or less, 34 mm or less, 33 mm or less, 32 mm or less, 31 mm or less, 30 mm or less, 29 mm or less, 28 mm or less, 27 mm or less, 26 mm or less, 25 mm or less, 24 mm or less, 23 mm or less, 22 mm or less, 21 mm or less, 20 mm or less, 19 mm or less, 18 mm or less, 17 mm or less, or 16 mm or less). The average characteristic dimension of the housing 206 can range from any of the minimum values described above to any of the maximum values described above. For example, the housing 206 can have an average characteristic dimension of from 15 mm to 75 mm (e.g., from 15 mm to 45 mm, from 45 mm to 75 mm, from 15 mm to 35 mm, from 35 mm to 55 mm, from 55 mm to 75 mm, from 15 mm to 70 mm, from 20 mm to 75 mm, from 20 mm to 70 mm, from 20 mm to 40 mm, or from 30 mm to 36 mm).

The interior cavity 208 can have any shape, such as a regular shape, an irregular shape, an isotropic shape, or an anisotropic shape. For example, the interior cavity 208 can be a polyhedron (e.g., a platonic solid, a prism, a pyramid), a cylinder, a hemicylinder, an elliptical cylinder, a hemi-elliptical cylinder, a cone, a semicone, etc. In some examples, the interior cavity 208 can have a cylindrical shape.

The interior cavity 208 has a longitudinal axis, a proximal end, and a distal end opposite and axially spaced apart from the proximal end. The interior cavity 208 has a length, the length being the dimension along the longitudinal axis from the proximal end to the distal end. The interior cavity 208 can have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the interior cavity 208 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the interior cavity 208 can range from any of the minimum values described above to any of the maximum values described above. For example, the interior cavity 208 can have an length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 30 mm to 150 mm, from 25 mm to 145 mm, from 30 mm to 145 mm, from 30 mm to 75 mm, or from 60 mm to 70 mm).

The interior cavity 208 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the interior cavity 208 can be substantially circular, ovate, ovoid, elliptic, triangular, rectangular, polygonal, etc.

The interior cavity 208 can have an average characteristic dimension. The term “characteristic dimension,” as used herein refers to the largest straight line distance between two points in the plane of the cross-sectional shape of the interior cavity 208. “Average characteristic dimension” and “mean characteristic dimension” are used interchangeably herein, and generally refer to the statistical mean characteristic dimension. For example, for a cylindrical interior cavity 208, the cross-sectional shape can be substantially circular and the average characteristic dimension can refer to the average diameter.

For example, the interior cavity 208 can have an average characteristic dimension of 5 mm or more (e.g., 6 mm or more, 7 mm or more, 8 mm or more, 9 mm or more, 10 mm or more, 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the interior cavity 208 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, or 6 mm or less). The average characteristic dimension of the interior cavity 208 can range from any of the minimum values described above to any of the maximum values described above. For example, the interior cavity 208 can have an average characteristic dimension of from 5 mm to 50 mm (e.g., from 5 mm to 30 mm, from 30 mm to 50 mm, from 5 mm to 20 mm, from 20 mm to 30 mm, from 30 mm to 40 mm, from 40 mm to 50 mm, from 10 mm to 50 mm, from 5 mm to 48 mm, from 10 mm to 48 mm, from 10 mm to 25 mm, or from 15 mm to 23 mm).

The first compartment 212 can have any shape, such as a regular shape, an irregular shape, an isotropic shape, or an anisotropic shape. For example, the first compartment 212 can be a polyhedron (e.g., a platonic solid, a prism, a pyramid), a cylinder, a hemicylinder, an elliptical cylinder, a hemi-elliptical cylinder, a cone, a semicone, etc. In some examples, the first compartment 212 can have a cylindrical shape.

In some examples, the first compartment 212 is disposed coaxially with the filter 210. The first compartment 212 has a longitudinal axis, with the proximal end 216 being opposite and axially spaced apart from the distal end 218. The first compartment 212 has a length, the length being the dimension along the longitudinal axis from the proximal end 216 to the distal end 218. The first compartment 212 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the first compartment 212 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the first compartment 212 can range from any of the minimum values described above to any of the maximum values described above. For example, the first compartment 212 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 60 mm to 75 mm, or from 60 mm to 70 mm).

The first compartment 212 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the first compartment 212 can be substantially circular, ovate, ovoid, elliptic, triangular, rectangular, polygonal, etc.

The first compartment 212 can have an average characteristic dimension. The term “characteristic dimension,” as used herein refers to the largest straight line distance between two points in the plane of the cross-sectional shape of the first compartment 212. “Average characteristic dimension” and “mean characteristic dimension” are used interchangeably herein, and generally refer to the statistical mean characteristic dimension. For example, for a cylindrical first compartment 212, the cross-sectional shape can be substantially circular and the average characteristic dimension can refer to the average diameter.

For example, the first compartment 212 can have an average characteristic dimension of mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the first compartment 212 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The average characteristic dimension of the first compartment 212 can range from any of the minimum values described above to any of the maximum values described above. For example, the first compartment 212 can have an average characteristic dimension of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm).

The filter 210 can comprise any suitable material, such as those known in the art. In some examples, the filter 210 can comprise a polymer, a composite material, a metal, or a combination thereof. In some examples, the filter 210 can comprise a polymer, such as nylon, polyester, polytetrafluoroethylene (PTFE), an acetal resin (e.g., Delrin®), polyether ether ketone (PEEK), polycarbonate, acrylonitrile butadiene styrene (ABS), poly(methyl methacrylate) (PMMA), polyvinyl chloride (PVC), an acrylic (e.g., a polyacrylic), derivatives thereof, or combinations thereof. In some examples, the filter 210 can comprise a metal, such as titanium, stainless steel, derivatives thereof, or combinations thereof.

In some examples, the filter 210 comprises a mesh filter. The mesh filter 210 can comprise a mesh with a plurality of openings. The plurality openings can for example, have an average size of 12 micrometers (microns, μm) or more (e.g., 13 μm or more, 14 μm or more, 15 μm or more, 20 μm or more, 25 μm or more, 30 μm or more, 35 μm or more, 40 μm or more, 45 μm or more, 50 μm or more, 55 μm or more, 60 μm or more, 65 μm or more, 70 μm or more, 75 μm or more, 80 μm or more, 85 μm or more, 90 μm or more, 95 μm or more, 100 μm or more, 105 μm or more, 110 μm or more, 115 μm or more, or 120 μm or more). In some examples, the plurality of openings can have an average size of 125 μm or less (e.g., 120 μm or less, 115 μm or less, 110 μm or less, 105 μm or less, 100 μm or less, 95 μm or less, 90 μm or less, 85 μm or less, 80 μm or less, 75 μm or less, 70 μm or less, 65 μm or less, 60 μm or less, 55 μm or less, 50 μm or less, 45 μm or less, 40 μm or less, 35 μm or less, 30 μm or less, 25 μm or less, 20 μm or less, or 15 μm or less). The average size of plurality of openings in the filter 210 can range from any of the minimum values described above to any of the maximum values described above. For example, the plurality of openings can have an average size of from 12 micrometers (microns, μm) to 125 μm (e.g., from 12 μm to 70 μm, from 70 μm to 125 μm, from 12 μm to 25 μm, from 25 μm to 50 μm, from 50 μm to 75 μm, from 75 μm to 100 μm, from 100 μm to 125 μm, from 15 μm to 125 μm, from 12 μm to 120 μm, or from 15 μm to 120 μm). The average size of the plurality of openings can, for example, be selected to optimize cleaning and concentration of the adipose tissue.

The filter 210 can have any shape, such as a regular shape, an irregular shape, an isotropic shape, or an anisotropic shape. For example, the filter 210 can be a polyhedron (e.g., a platonic solid, a prism, a pyramid), a cylinder, a hemicylinder, an elliptical cylinder, a hemi-elliptical cylinder, a cone, a semicone, etc. In some examples, the filter 210 can have a cylindrical shape.

The filter 210 has a longitudinal axis, a proximal end, and a distal end opposite and axially spaced apart from the proximal end. The filter 210 has a length, the length being the dimension along the longitudinal axis from the proximal end to the distal end.

The filter 210 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the filter 210 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the filter 210 can range from any of the minimum values described above to any of the maximum values described above. For example, the filter 210 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 40 mm to 60 mm, or from 48 mm to 56 mm). For example, the length of the filter 210 can be selected in view of the length of the interior cavity 208.

The filter 210 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the filter 210 can be substantially circular, ovate, ovoid, elliptic, triangular, rectangular, polygonal, etc.

The filter 210 can have an average characteristic dimension. The term “characteristic dimension,” as used herein refers to the largest straight line distance between two points in the plane of the cross-sectional shape of the filter 210. “Average characteristic dimension” and “mean characteristic dimension” are used interchangeably herein, and generally refer to the statistical mean characteristic dimension. For example, for a cylindrical filter 210, the cross-sectional shape can be substantially circular and the average characteristic dimension can refer to the average diameter.

For example, the filter 210 can have an average characteristic dimension of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the filter 210 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The average characteristic dimension of the filter 210 can range from any of the minimum values described above to any of the maximum values described above. For example, the filter 210 can have an average characteristic dimension of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm). The average characteristic dimension of the filter 210 can, for example, be selected in view of the average characteristic dimension of the interior cavity 208.

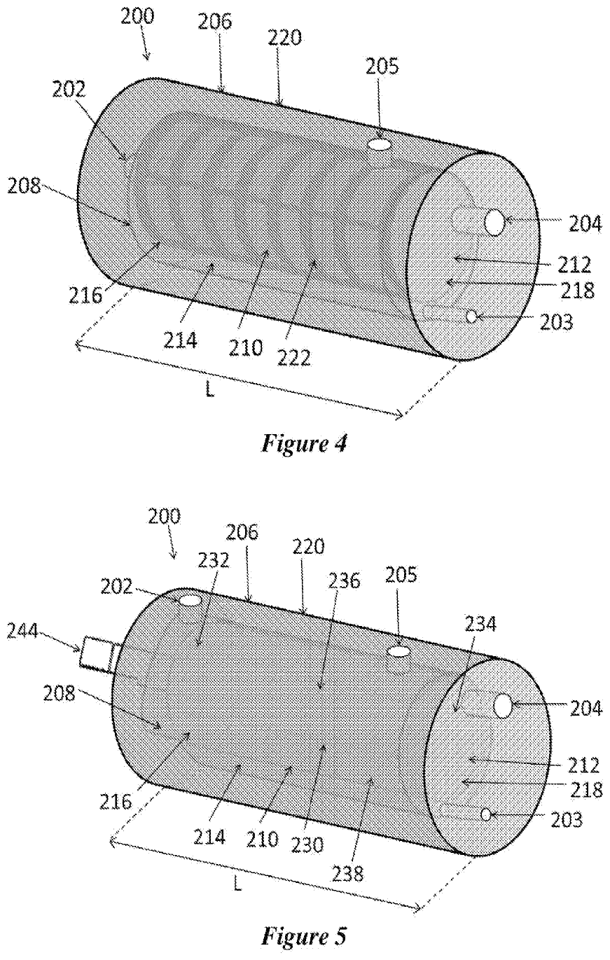

Referring now to FIG. 4, in some examples, the adipose separation module 200 further comprises a filter cage 222 disposed circumferentially around and coaxially with the filter 210 within the interior cavity 208. The filter cage 222 can, for example, provide structural support and/or rigidity to the filter 210.

The filter cage 222 can comprise any suitable material, such as those known in the art. In some examples, the filter cage 222 can comprise a polymer, a composite material, a metal, or a combination thereof.

The filter cage 222 can have any shape, such as a regular shape, an irregular shape, an isotropic shape, or an anisotropic shape. For example, the filter cage 222 can be a polyhedron (e.g., a platonic solid, a prism, a pyramid), a cylinder, a hemicylinder, an elliptical cylinder, a hemi-elliptical cylinder, a cone, a semicone, etc. In some examples, the filter cage 222 can have a cylindrical shape. The shape of the filter case 222 can be selected, for example, in view of the shape of the filter 210 and/or the interior cavity 208.

The filter cage 222 has a longitudinal axis, a proximal end, and a distal end opposite and axially spaced apart from the proximal end. The filter cage 222 has a length, the length being the dimension along the longitudinal axis from the proximal end to the distal end.

The filter cage 222 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the filter cage 222 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the filter cage 222 can range from any of the minimum values described above to any of the maximum values described above. For example, the filter cage 222 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 40 mm to 60 mm, or from 48 mm to 56 mm). The length of the filter case 222 can be selected, for example, in view of the length of the filter 210 and/or the interior cavity 208.

The filter cage 222 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the filter cage 222 can be substantially circular, ovate, ovoid, elliptic, triangular, rectangular, polygonal, etc.

The filter cage 222 can have an average characteristic dimension. The term “characteristic dimension,” as used herein refers to the largest straight line distance between two points in the plane of the cross-sectional shape of the filter cage 222. “Average characteristic dimension” and “mean characteristic dimension” are used interchangeably herein, and generally refer to the statistical mean characteristic dimension. For example, for a cylindrical filter cage 222, the cross-sectional shape can be substantially circular and the average characteristic dimension can refer to the average diameter.

For example, the filter cage 222 can have an average characteristic dimension of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the filter cage 222 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The average characteristic dimension of the filter cage 222 can range from any of the minimum values described above to any of the maximum values described above. For example, the filter cage 222 can have an average characteristic dimension of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm). The average characteristic dimension of the filter cage 222 can be selected, for example, in view of the average characteristic dimension of the filter 210 and/or the interior cavity 208.

Referring now to FIG. 5, in some examples, the adipose separation module 200 further comprises: a rotary implement 230 having longitudinal axis, a proximal end 232, and a distal end 234 axially spaced apart from the proximal end, the rotary implement 230 comprising a central shaft 236 and a blade 238 extending from the central shaft 236; wherein the filter is configured to be disposed circumferentially around and coaxially with the rotary implement 230 within the interior cavity 208, such that the rotary implement 230 is configured to be rotatably disposed within the first compartment 212 with the proximal end 232 of the rotary implement 230 disposed towards the proximal end 216 of the first compartment 212 and the distal end 234 of the rotary implement 230 being disposed towards the distal end 218 of the first compartment 212; and wherein the rotary implement 230 is configured to agitate the mixture within the first compartment 212 via rotation of the rotary implement 230.

The rotary implement 230 can comprise any suitable material, such as those known in the art. In some examples, the rotary implement 230 can comprise a polymer, a composite material, a metal, or a combination thereof. In some examples, the rotary implement 230 can comprise polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof. In some examples, the rotary implement 230 can comprise polycarbonate. In some examples, the rotary implement 230 can comprise a metal, such as titanium, stainless steel, derivatives thereof, or combinations thereof.

The rotary implement 230 has a length, the length being the dimension along the longitudinal axis from the proximal end to the distal end. The rotary implement 230 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the rotary implement 230 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the rotary implement 230 can range from any of the minimum values described above to any of the maximum values described above. For example, the rotary implement 230 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 40 mm to 60 mm, or from 48 mm to 56 mm). The length of the rotary implement 230 can be selected, for example, in view of the length of the filter 210 and/or the interior cavity 208.

For example, the central shaft 236 of the rotary implement 230 can have a diameter of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the central shaft 236 of the rotary implement 230 can have a diameter of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The diameter of the central shaft 236 of the rotary implement 230 can range from any of the minimum values described above to any of the maximum values described above. For example, the central shaft 236 of the rotary implement 230 can have a diameter of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm).

In some examples, the blade 238 of the rotary implement 230 has an edge and the edge and the filter 210 are radially spaced apart from each other by a distance of 0 micrometers (microns, μm) or more (e.g., 1 μm or more, 2 μm or more, 3 μm or more, 4 μm or more, 5 μm or more, 10 μm or more, 15 μm or more, 20 μm or more, 25 μm or more, 30 μm or more, 40 μm or more, 50 μm or more, 75 μm or more, 100 μm or more, 125 μm or more, 150 μm or more, 175 μm or more, 200 μm or more, 250 μm or more, 300 μm or more, 350 μm or more, 400 μm or more, 450 μm or more, 500 μm or more, 600 μm or more, 700 μm or more, 800 μm or more, 900 μm or more, 1 millimeters (mm) or more, 1.25 mm or more, 1.5 mm or more, or 1.75 mm or more). In some examples, the edge and the filter 210 are radially spaced apart from each other by a distance of 2 mm or less (e.g., 1.75 mm or less, 1.5 mm or less, 1.25 mm or less, 1 mm or less, 900 μm or less, 800 μm or less, 700 μm or less, 600 μm or less, 500 μm or less, 450 μm or less, 400 μm or less, 350 μm or less, 300 μm or less, 250 μm or less, 200 μm or less, 175 μm or less, 150 μm or less, 125 μm or less, 100 μm or less, 75 μm or less, 50 μm or less, 40 μm or less, 30 μm or less, 25 μm or less, 20 μm or less, 15 μm or less, 10 μm or less, 5 μm or less, 4 μm or less, 3 μm or less, 2 μm or less, or 1 μm or less). The distance that the edge and the filter 210 are radially spaced apart from each other can range from any of the minimum values described above to any of the maximum values described above. For example, the edge and the filter 210 can be radially spaced apart from each other by a distance of from 0 μm to 2 mm (e.g., from 0 μm to 100 μm, from 100 μm to 2 mm, from 0 μm to 10 μm, from 10 μm to 100 μm, from 100 μm to 1 mm, from 1 mm to 2 mm, from 0 μm to 1.75 mm, from 1 μm to 2 mm, or from 1 μm to 1.75 mm). The distance can, for example, be selected to optimize the cleaning and concentration of the adipose tissue and/or to minimize damage to the adipose tissue.

The rotary implement 230 has an outer diameter as measured to the edge of the blade 238 (e.g., edge to edge for a helical blade 238). For example, the rotary implement 230 can have an outer diameter of 5 mm or more (e.g., 6 mm or more, 7 mm or more, 8 mm or more, 9 mm or more, 10 mm or more, 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the rotary implement 230 can have an outer diameter of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, or 6 mm or less). The outer diameter of the rotary implement 230 can range from any of the minimum values described above to any of the maximum values described above. For example, the rotary implement 230 can have an outer diameter of from 5 mm to 50 mm (e.g., from 5 mm to 30 mm, from 30 mm to 50 mm, from 5 mm to 20 mm, from 20 mm to 30 mm, from 30 mm to 40 mm, from 40 mm to 50 mm, from 10 mm to 50 mm, from 5 mm to 48 mm, from 10 mm to 48 mm, from 10 mm to 25 mm, or from 10 mm to 15 mm).

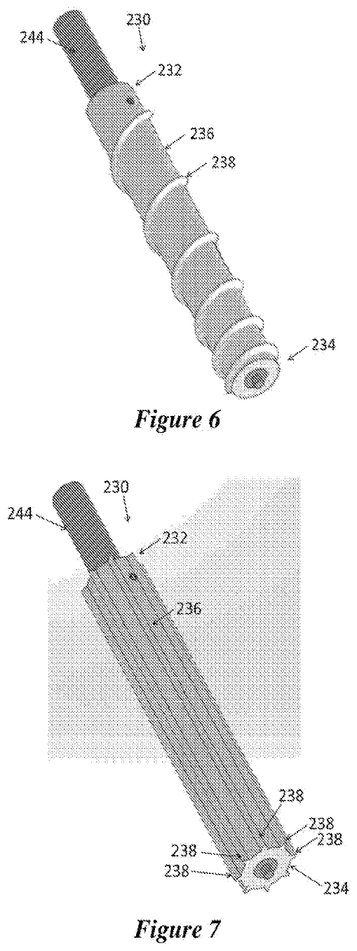

The rotary implement 230 can have any suitable configuration. Example rotary implements 230 are shown in FIG. 6 to FIG. 10.

In some examples, the blade 238 is helically disposed about the central shaft 236.

Referring now to FIG. 6, in some examples, the blade 238 is helically disposed about the central shaft 236 with a variable pitch.

Referring now to FIG. 7, in some examples, the blade 238 comprises a plurality of blades 238, each of the blades 238 extending radially from the central shaft 236 and being disposed circumferentially about the central shaft 236. An example adipose filtration modules using the rotary implement 230 as shown in FIG. 7 is shown in FIG. 54.

In some examples, the rotary implement 230 comprises a first section 240 and a second section 242, the first section 240 extending along a first portion of the central shaft 236 towards the proximal end 232 of the rotary implement 230 and the second section 242 being adjacent the first section and extending along a second portion of the central shaft 236, wherein the first section 240 has a first blade 238A extending from the central shaft 236 and the second section 242 has a second blade 238B extending from the central shaft 236.

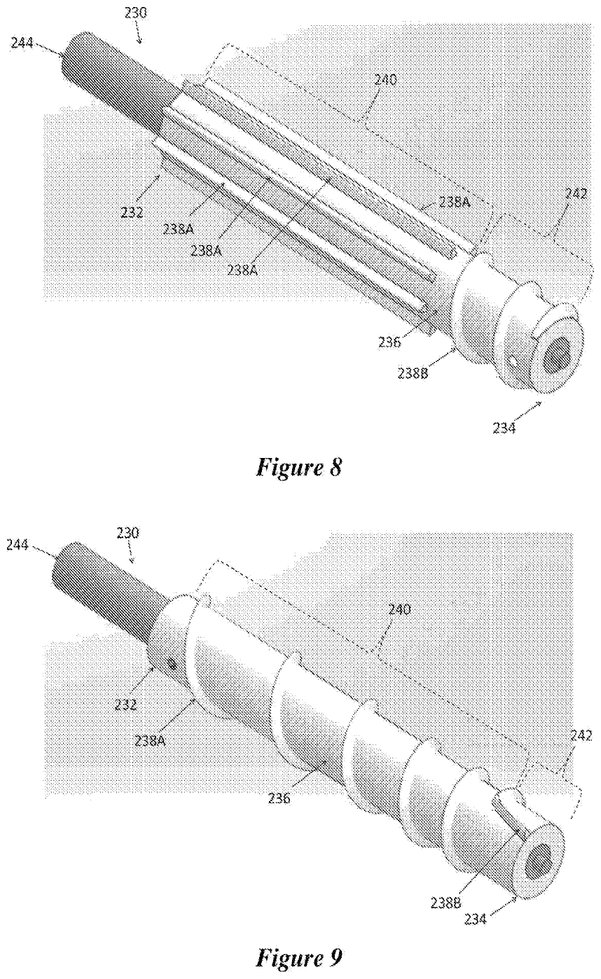

Referring now to FIG. 8, in some examples, the second blade 238B is helically disposed about the central shaft 236 and the first blade 238A comprises a plurality of first blades 238A, each of the first blades 238A extending radially from the central shaft 236 and being disposed circumferentially about the central shaft 236.

Referring now to FIG. 9, in some examples, the first blade 238A is helically disposed about the central shaft 236 and the second blade 238B is a single blade extending radially from the central shaft 236, wherein the first section 240 borders the second section 242 and the first blade 238A is joined to the second blade 238B at the border between the first section 240 and the second section 242. In some examples, the first blade 238A is helically disposed about the central shaft 236 with a variable pitch.

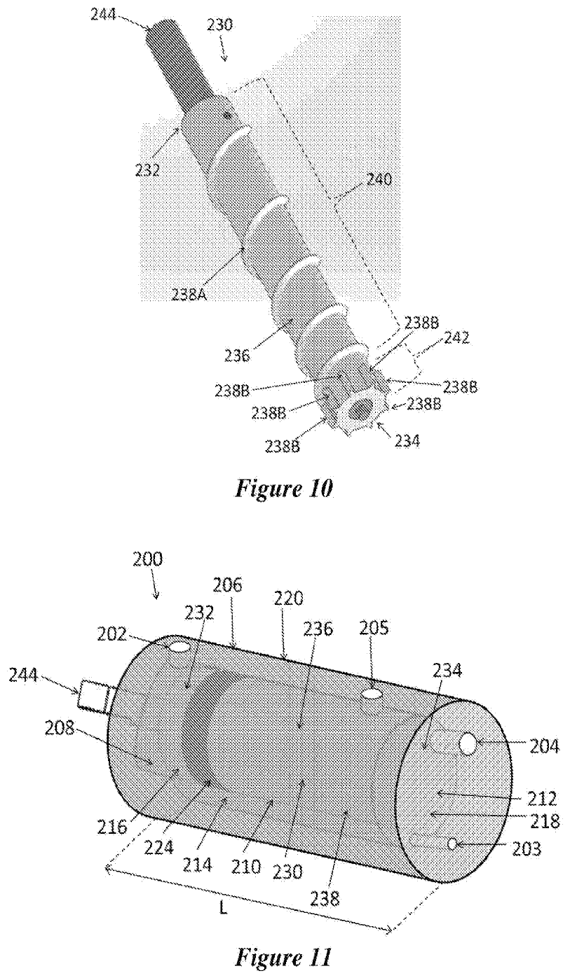

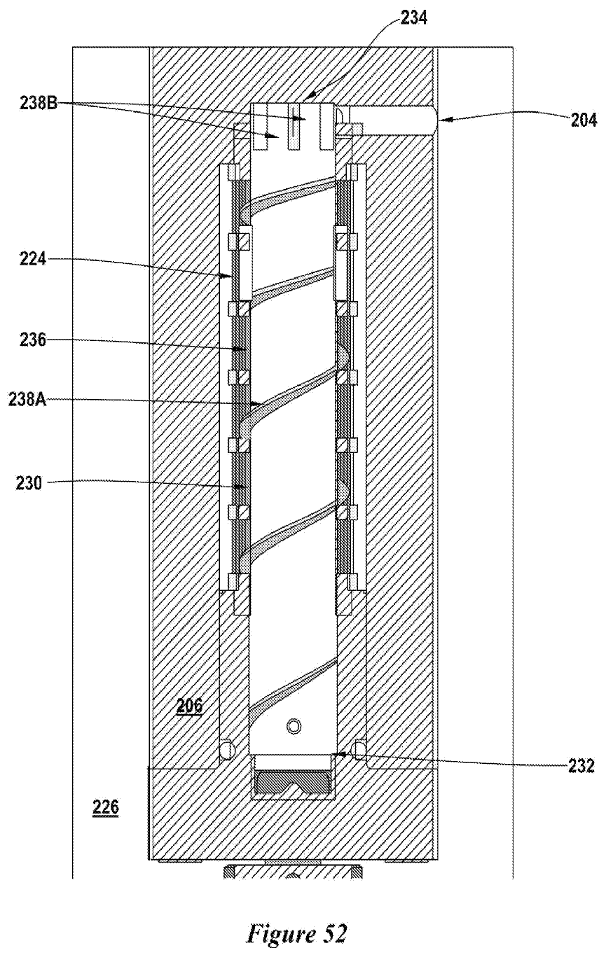

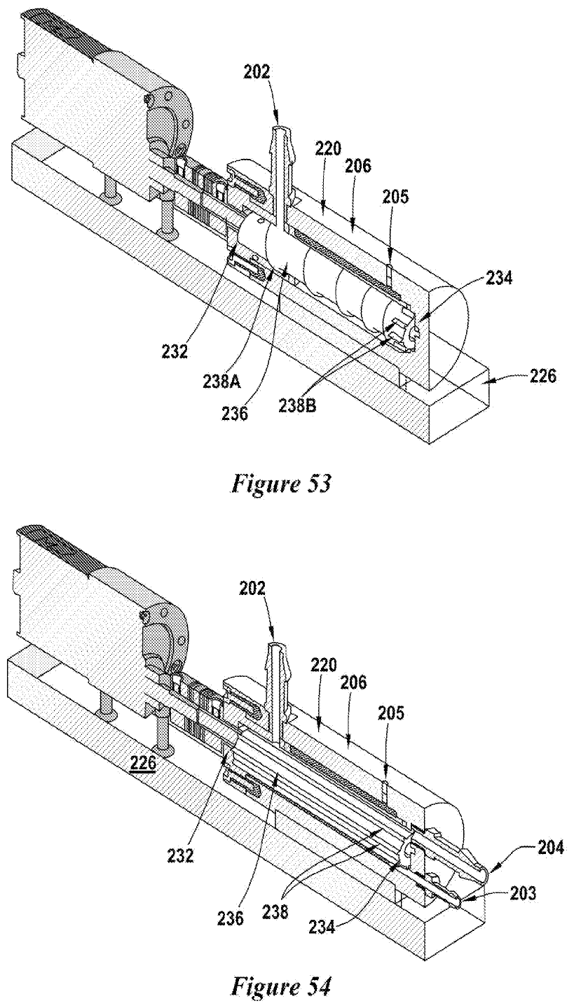

Referring now to FIG. 10, in some examples, the first blade 238A is helically disposed about the central shaft 236 and the second blade 238B comprises a plurality of second blades 238B, each of the second blades 238B extending radially from the central shaft 236 and being disposed circumferentially about the central shaft 236. Example adipose filtration modules 200 including the rotary implement 230 as shown in FIG. 10 are shown in FIG. 52 and FIG. 53. In certain examples, the outlet 204 can be located at a 90° angle relative to the longitudinal axis of the rotary implement 230, as shown in FIG. 52.

In some examples, the adipose separation module 200 further comprises a rod 244 and wherein the central shaft 236 of the rotary implement 230 is disposed circumferentially around and coaxially with the rod 244.

The rod 244 can comprise any suitable material, such as those known in the art. In some examples, the rod 244 can comprise a polymer, a composite material, a metal, or a combination thereof. In some examples, the rod 244 can comprise a metal, such as titanium, stainless steel, derivatives thereof, or combinations thereof.

Referring now to FIG. 11, in some examples, the adipose separation module 200 further comprises a sliding ring 224, the sliding ring 224 being slidably disposed between the rotary implement 230 and an inner surface of the filter 210, such that the sliding ring 224 is disposed circumferentially around and coaxially with the rotary implement 230 and the inner surface of the filter 210 is disposed circumferentially around and coaxially with the sliding ring 224. The sliding ring 224 can be configured, for example, to slide axially to clear the inner surface of the filter 210.

The sliding ring 244 can be configured to be coupled to a means for axially sliding the sliding ring 244.

The means for sliding the sliding ring 244 can be a manual means or an automated means. Examples of suitable means for sliding the sliding ring 244 include, but are not limited to, handles, actuators, magnetic coupling, and combinations thereof. In some examples, the means for sliding the sliding ring 244 can comprise an actuator coupled to the sliding ring 224. In some examples, the kit further comprises the means for sliding the sliding ring 244.

The sliding ring 224 can comprise any suitable material, such as those known in the art. In some examples, the sliding ring 224 can comprise a polymer, a composite material, a metal, or a combination thereof. In some examples, the sliding ring 224 can comprise a polymer, such as an acetal resin (e.g., Delrin®), polytetrafluoroethylene (PTFE), polyether ether ketone (PEEK), polycarbonate, acrylonitrile butadiene styrene (ABS), poly(methyl methacrylate) (PMMA), polyvinyl chloride (PVC), an acrylic (e.g., a polyacrylic), nylon, polyester, derivatives thereof, or combinations thereof. In some examples, the sliding ring 224 can comprise a magnetic material.

The sliding ring 224 has a longitudinal axis, a proximal end, and a distal end opposite and axially spaced apart from the proximal end. The sliding ring 224 has a length, the length being the dimension along the longitudinal axis from the proximal end to the distal end. The sliding ring 224 can have a length of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the sliding ring 224 can have a length of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The length of the sliding ring 224 can range from any of the minimum values described above to any of the maximum values described above. For example, the sliding ring 224 can have a length of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 10 mm to 15 mm).

The sliding ring 224 can, for example, have an outer diameter of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the sliding ring 224 can have an outer diameter of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The outer diameter of the sliding ring 224 can range from any of the minimum values described above to any of the maximum values described above. For example, the sliding ring 224 can have an outer diameter of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 10 mm to 15 mm).

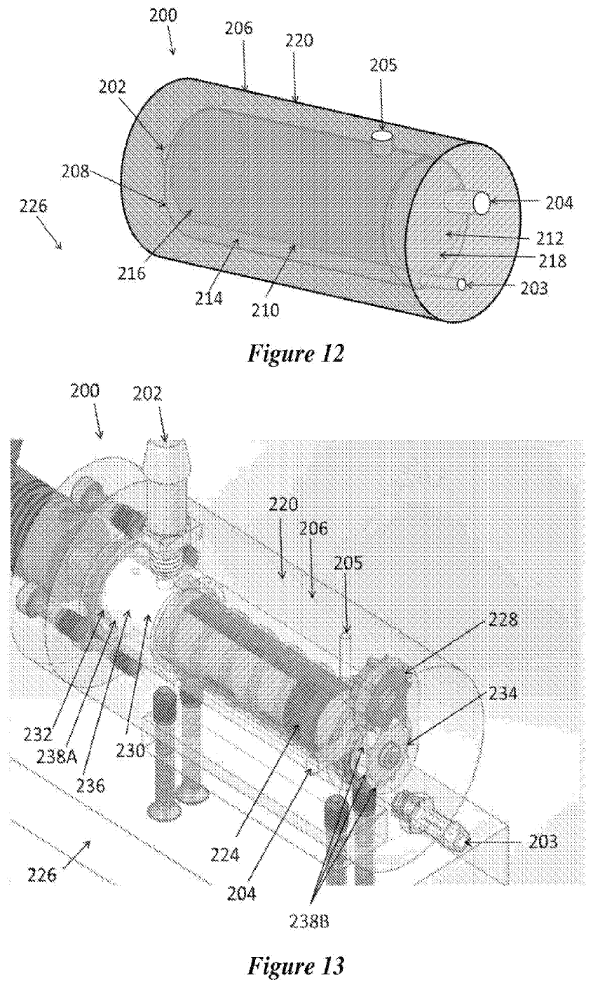

Referring now to FIG. 12, in some examples, the adipose separation module 200 is disposed on a platform 226. The adipose separation module 200 can, for example, be attached to the platform 226 using adhesives, bolts, screws, clips, or any other mechanical or chemical fastener known in the art.

The platform 226 can comprise any suitable material, such as those known in the art. In some examples, the platform 226 can comprise a polymer, a composite material, a metal, or a combination thereof. In some examples, the platform 226 can comprise a polymer, such as polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof.

In some examples, the rotary implement 230 is configured to be coupled to a means for rotating 228 the rotary implement 230, wherein the means for rotating 228 the rotary implement 230 is configured to rotate the rotary implement 230 within the first compartment 212.

The means for rotating 228 the rotary implement 230 can comprise any suitable means, such as those known in the art. The means for rotating 228 the rotary implement can be a manual means or an automated means. Examples of suitable means for rotating 228 the rotary implement include, but are not limited to, cranks, motors, gears, and combinations thereof. In some examples, the means for rotating 228 the rotary implement 230 can comprise a motor, such as variable speed motor. In some examples, the kit 100 further comprises a means for rotating 228 the rotary implement 230 of the adipose separation module 200.

For example, the means for rotating 228 the rotary implement 230 can comprise a variable speed motor, such as a gear motor with a controllable speed. For example, the motor 228 can have a speed of 2 RPM or more (e.g., 3 RPM or more, 4 RPM or more, 5 RPM or more, 6 RPM or more, 7 RPM or more, 8 RPM or more, 9 RPM or more, 10 RPM or more, 11 RPM or more, 12 RPM or more, 13 RPM or more, 14 RPM or more, 15 RPM or more, 16 RPM or more, 17 RPM or more, 18 RPM or more, 19 RPM or more, 20 RPM or more, 21 RPM or more, 22 RPM or more, 23 RPM or more, or 24 RPM or more). In some examples, the motor 228 can have a speed of 25 RPM or less (e.g., 24 RPM or less, 23 RPM or less, 22 RPM or less, 21 RPM or less, 20 RPM or less, 19 RPM or less, 18 RPM or less, 17 RPM or less, 16 RPM or less, 15 RPM or less, 14 RPM or less, 13 RPM or less, 12 RPM or less, 11 RPM or less, 10 RPM or less, 9 RPM or less, 8 RPM or less, 7 RPM or less, 6 RPM or less, 5 RPM or less, 4 RPM or less, or 3 RPM or less). The speed of the motor 228 can range from any of the minimum values described above to any of the maximum values described above. For example, the motor 228 can have a speed of from 2 RPM to 25 RPM (e.g., from 2 to 13 RPM, from 13 to 25 RPM, from 2 to 5 RPM, from 5 to 10 RPM, from 10 to 15 RPM, from 15 to 20 RPM, from 20 to 25 RPM, from 3 to 25 RPM, from 4 to 25 RPM, from 5 to 25 RPM, from 6 to 25 RPM, from 8 to 25 RPM, from 10 to 25 RPM, from 15 to 25 RPM, from 2 to 24 RPM, from 2 to 23 RPM, from 2 to 22 RPM, from 2 to 21 RPM, from 2 to 20 RPM, from 2 to 18 RPM, from 2 to 15 RPM, from 2 to 10 RPM, from 3 to 24 RPM, from 5 to 20 RPM, from 10 to 20 RPM, or from 15 to 20 RPM).

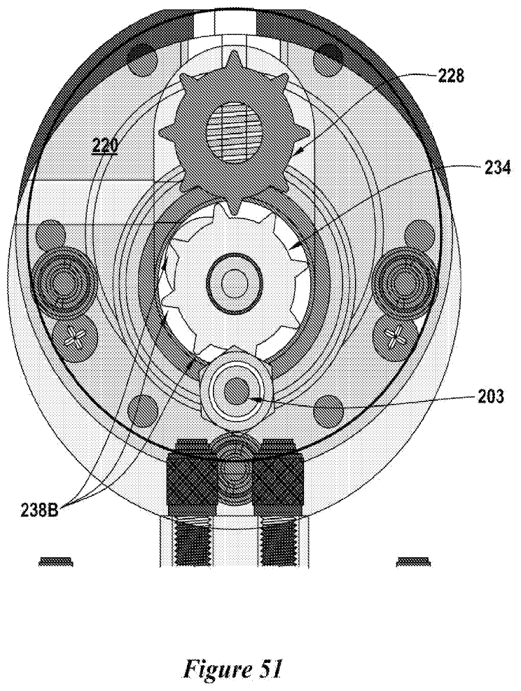

Referring now to FIG. 13, in some examples, the means for rotating 228 the rotary implement 230 of the adipose separation module 200 comprises a gear pump disposed inside the housing 206 and coupled to the rotary implement 230 of the adipose separation module 200. An example adipose filtration modules 200 including a gear pump is shown in FIG. 51.

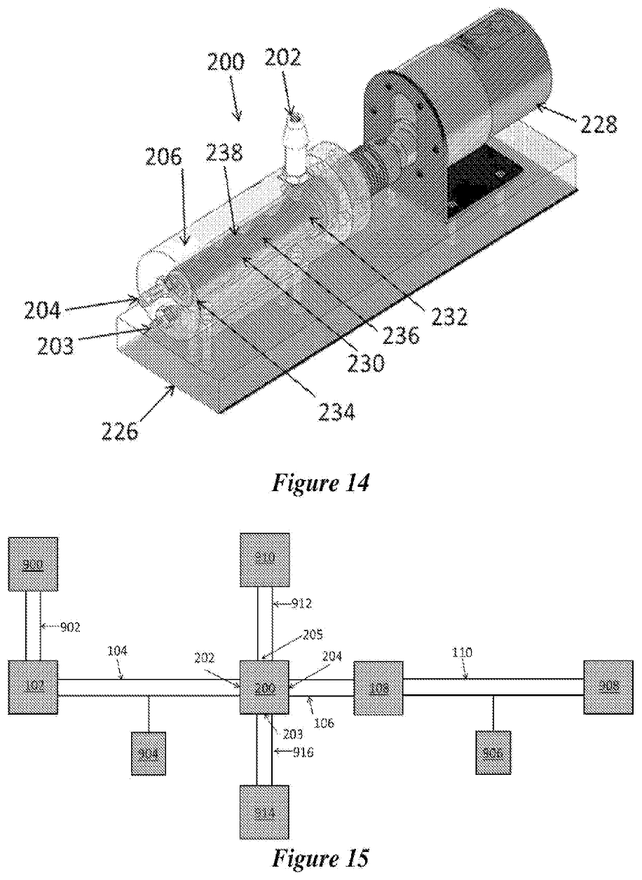

Referring now to FIG. 14, in some examples, the means for rotating 228 the rotary implement 230 of the adipose separation module 200 comprises a motor disposed outside the housing 206 and coupled to the central shaft 236 and/or the rod 244 of the rotary implement 230 of the adipose separation module 200.

In some examples, the adipose separation module 200 further comprises a port 203, wherein the port 230 is defined by the housing 206 and is fluidly connected to the second compartment 214, for example at or near the distal end of the second compartment 214.

The port 203 can, for example, have an inner diameter of 1 mm or more (e.g., 1.5 mm or more, 2 mm or more, 2.5 mm or more, 3 mm or more, 3.5 mm or more, 4 mm or more, 4.5 mm or more, 5 mm or more, 5.5 mm or more, 6 mm or more, 6.5 mm or more, 7 mm or more, 7.5 mm or more, 8 mm or more, 8.5 mm or more, 9 mm or more, 9.5 mm or more, 10 mm or more, 10.5 mm or more, 11 mm or more, or 11.5 mm or more). In some examples, the port 203 can have an inner diameter of 12 mm or less (e.g., 11.5 mm or less, 11 mm or less, 10.5 mm or less, 10 mm or less, 9.5 mm or less, 9 mm or less, 8.5 mm or less, 8 mm or less, 7.5 mm or less, 7 mm or less, 6.5 mm or less, 6 mm or less, 5.5 mm or less, 5 mm or less, 4.5 mm or less, 4 mm or less, 3.5 mm or less, 3 mm or less, 2.5 mm or less, 2 mm or less, or 1.5 mm or less). The inner diameter of the port 203 can range from any of the minimum values described above to any of the maximum values described above. For example, the port 203 can have an inner diameter of from 1 mm to 12 mm (e.g., from 1 mm to 6 mm, from 6 mm to 12 mm, from 1 mm to 4 mm, from 4 mm to 8 mm, from 8 mm to 12 mm, from 2 mm to 12 mm, from 1 mm to 11 mm, or from 2 mm to 11 mm).

Referring now to FIG. 15, in some examples, the port 203 of the adipose separation module 200 is configured to be fluidly connected to an effluent receptacle 914, for example via a sixth tube 916, the effluent receptacle 914 being configured to receive the effluent from the port 203 of the adipose separation module 200.

For example, the port 203 of the adipose separation module 200 can be configured to be fluidly connected to the sixth tube 916, wherein the sixth tube 916 is configured to fluidly connect the port 203 of the adipose separation module 200 to an effluent receptacle 914. In some examples, the kit 100 further comprises an effluent receptacle 914 configured to be fluidly connected to the port 203 of the adipose separation module 200. In some examples, the kit 100 further comprises a sixth tube 916 configured to fluidly connect the port 203 of the adipose separation module to an effluent receptacle 914. In some examples, the kit 100 further comprises a sixth tube 916 and an effluent receptacle 914, wherein the sixth tube 916 is configured to fluidly connect the port 203 of the adipose separation module 200 to the effluent receptacle 914, the effluent receptacle 914 being configured to receive the effluent from the port 203 of the adipose separation module 200.

The effluent receptacle 914 can, for example, comprise any suitable container for containing the effluent, such as those known in the art. In some examples, the effluent receptacle 914 can comprise a flexible polymer collection bag, vacutainer, or vacuum canister. In some examples, the effluent receptacle 914 can comprise a disposable medical collection bag. In some examples, the effluent receptacle 914 containing the effluent can be disposable as biohazard waste. The effluent receptacle 914 can, for example, comprise any suitable material, such as those known in the art. For example, the effluent receptacle 914 can comprise a polymer.

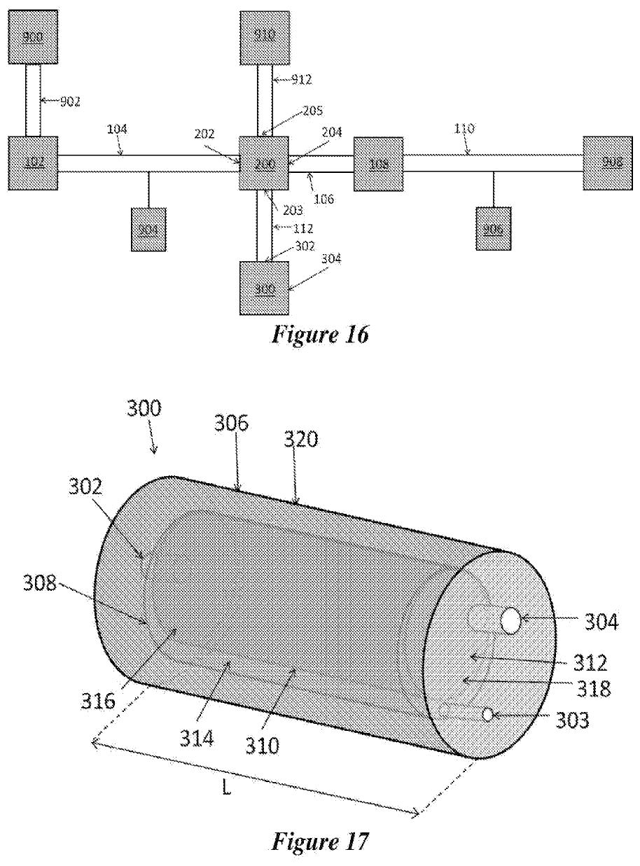

Referring now to FIG. 16, in some examples, the kit 100 further comprises a seventh tube 112 and an effluent separation module 300 having an inlet 302 and an outlet 304, wherein the seventh tube 112 is configured to fluidly connect the inlet 302 of the effluent separation module 300 to the port 203 of the adipose separation module 200, the effluent separation module 300 being configured to receive the effluent from the port 203 of the adipose separation module 200 and separate a desired component from the effluent.

The inlet 302 of the effluent separation module 300 can, for example, have an inner diameter of 1 millimeter (mm) or more (e.g., 1.25 mm or more, 1.5 mm or more, 1.75 mm or more, 2 mm or more, 2.25 mm or more, 2.5 mm or more, 2.75 mm or more, 3 mm or more, 3.25 mm or more, 3.5 mm or more, 3.75 mm or more, 4 mm or more, 4.25 mm or more, 4.5 mm or more, 4.75 mm or more, 5 mm or more, 5.25 mm or more, 5.5 mm or more, or 5.75 mm or more). In some examples, the inlet 302 of the effluent separation module 300 can have an inner diameter of 6 mm or less (e.g., 5.75 mm or less, 5.5 mm or less, 5.25 mm or less, 5 mm or less, 4.75 mm or less, 4.5 mm or less, 4.25 mm or less, 4 mm or less, 3.75 mm or less, 3.5 mm or less, 3.25 mm or less, 3 mm or less, 2.75 mm or less, 2.5 mm or less, 2.25 mm or less, 2 mm or less, 1.75 mm or less, 1.5 mm or less, or 1.25 mm or less). The inner diameter of inlet 302 of the effluent separation module 300 can range from any of the minimum values described above to any of the maximum values described above. For example, the inlet 302 of the effluent separation module 200 can have an inner diameter of from 1 millimeter (mm) to 6 mm (e.g., from 1 mm to 3.5 mm, from 3.5 mm to 6 mm, from 1 mm to 2 mm, from 2 mm to 3 mm, from 3 mm to 4 mm, from 4 mm to 5 mm, from 5 mm to 6 mm, from 1 mm to 5.5 mm, from 1.5 mm to 5.5 mm, from 1.5 mm to 5.5 mm, from 2 mm to 6 mm, from 3 mm to 6 mm, from 4 mm to 6 mm, or from 5.25 mm to 5.5 mm).

The outlet 304 of the effluent separation module 300 can, for example, have an inner diameter of 1 millimeter (mm) or more (e.g., 1.25 mm or more, 1.5 mm or more, 1.75 mm or more, 2 mm or more, 2.25 mm or more, 2.5 mm or more, 2.75 mm or more, 3 mm or more, 3.25 mm or more, 3.5 mm or more, 3.75 mm or more, 4 mm or more, 4.25 mm or more, 4.5 mm or more, 4.75 mm or more, 5 mm or more, 5.25 mm or more, 5.5 mm or more, or 5.75 mm or more). In some examples, the outlet 304 of the effluent separation module 200 can have an inner diameter of 6 mm or less (e.g., 5.75 mm or less, 5.5 mm or less, 5.25 mm or less, 5 mm or less, 4.75 mm or less, 4.5 mm or less, 4.25 mm or less, 4 mm or less, 3.75 mm or less, 3.5 mm or less, 3.25 mm or less, 3 mm or less, 2.75 mm or less, 2.5 mm or less, 2.25 mm or less, 2 mm or less, 1.75 mm or less, 1.5 mm or less, or 1.25 mm or less). The inner diameter of outlet of the effluent separation module 200 can range from any of the minimum values described above to any of the maximum values described above. For example, the outlet 204 of the effluent separation module 200 can have an inner diameter of from 1 millimeter (mm) to 6 mm (e.g., from 1 mm to 3.5 mm, from 3.5 mm to 6 mm, from 1 mm to 2 mm, from 2 mm to 3 mm, from 3 mm to 4 mm, from 4 mm to 5 mm, from 5 mm to 6 mm, from 1 mm to 5.5 mm, from 1.5 mm to 5.5 mm, from 1.5 mm to 5.5 mm, from 2 mm to 6 mm, from 3 mm to 6 mm, from 4 mm to 6 mm, or from 5.25 mm to 5.5 mm).

In some examples, the effluent separation module 300 comprises a membrane filter, the membrane filter being configured to separate the desired component from other components in the effluent by passing the desired component through the membrane filter and blocking the passage of the other components through the membrane filter. The desired component can, for example, comprise platelets, plasma, platelet-rich plasma (PRP), stem cells, a protein, or a combination thereof.

Referring now to FIG. 17, in some examples, the effluent separation module 300 further comprises: a housing 306 defining an interior cavity 308, the inlet 302, and the outlet 304. In some examples, the effluent separation module 300 further comprises a filter 310 configured to be disposed within the interior cavity 308, such that the filter 310 is configured to define a first compartment 312 and a second compartment 314 within the interior cavity 308, the first compartment 312 being a portion of the interior cavity 308 encompassed by the filter 310 and the second compartment 314 being a portion of the interior cavity 308 outside the filter 310; wherein the first compartment 312 has a proximal end 316 and a distal end 318, the inlet 302 of the effluent separation module 300 being fluidly connected to the first compartment 312 at or near the proximal end 316 of the first compartment 312, and the outlet 304 of the effluent separation module 300 being fluidly connected to the first compartment 312 at or near the distal end 318 of the first compartment 312.

In some examples, the filter 310 is configured to separate the desired component from the other components in the effluent by passing the other components from the effluent through the filter 310 into the second compartment 314 as the effluent is transported through the housing 306, thereby concentrating the desired component within the first compartment 312 and forming a waste solution in the second compartment 314, the waste solution comprising the other components from the effluent. The desired component can, for example, comprise platelets, plasma, platelet-rich plasma (PRP), stem cells, a protein, or a combination thereof. In some examples, the desired component can be reintroduced to the concentrated adipose tissue. In some examples, the desired component can be used for a different medical procedure.

The housing 306 can comprise any suitable material, such as those known in the art. Examples of suitable materials for the housing 306 include, but are not limited to, polymers, such as transparent and semi-transparent polymers. In some examples, the housing 306 can comprise polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof. In some examples, the housing 306 can comprise polycarbonate.

The housing 306 can have any shape, such as a regular shape, an irregular shape, an isotropic shape, or an anisotropic shape. For example, the housing 306 can be a polyhedron (e.g., a platonic solid, a prism, a pyramid), a cylinder, a hemicylinder, an elliptical cylinder, a hemi-elliptical cylinder, a cone, a semicone, etc. In some examples, the housing 306 can have a cylindrical shape.

The housing 306 has a longitudinal axis, a proximal end, and a distal end opposite and axially spaced apart from the proximal end. The housing 306 has a length, the length being the dimension along the longitudinal axis from the proximal end to the distal end (e.g., “L” in FIG. 17). In some examples, the housing 306 has a length of 50 mm or more (e.g., 55 mm or more, 60 mm or more, 65 mm or more, 70 mm or more, 75 mm or more, 80 mm or more, 85 mm or more, 90 mm or more, 95 mm or more, 100 mm or more, 110 mm or more, 120 mm or more, 130 mm or more, 140 mm or more, 150 mm or more, 160 mm or more, 170 mm or more, 180 mm or more, or 190 mm or more). In some example, the housing 306 has a length of 200 mm or less (e.g., 190 mm or less, 180 mm or less, 170 mm or less, 160 mm or less, 150 mm or less, 140 mm or less, 130 mm or less, 120 mm or less, 110 mm or less, 100 mm or less, 95 mm or less, 90 mm or less, 85 mm or less, 80 mm or less, 75 mm or less, 70 mm or less, 65 mm or less, 60 mm or less, or 55 mm or less). The length of the housing 306 can range from any of the minimum values described above to any of the maximum values described above. For example, the housing 306 can have a length of from 50 mm to 200 mm (e.g., from 50 mm to 125 mm, from 125 mm to 200 mm, from 50 mm to 100 mm, from 100 mm to 150 mm, from 150 mm to 200 mm, from 50 mm to 175 mm, from 75 mm to 200 mm, from 75 mm to 174 mm, from 50 mm to 150 mm, from 60 mm to 90 mm, from 70 mm to 80 mm, or from 75 mm to 80 mm). In some examples, the housing 306 can have a length of from 75 mm to 80 mm.

The housing 306 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the housing 306 can be substantially circular, ovate, ovoid, elliptic, triangular, rectangular, polygonal, etc.

The housing 306 can have an average characteristic dimension. The term “characteristic dimension,” as used herein refers to the largest straight line distance between two points in the plane of the cross-sectional shape of the housing 306. “Average characteristic dimension” and “mean characteristic dimension” are used interchangeably herein, and generally refer to the statistical mean characteristic dimension. For example, for a cylindrical housing 306, the cross-sectional shape can be substantially circular and the average characteristic dimension can refer to the average diameter.

For example, the housing 306 can have an average characteristic dimension of 15 mm or more (e.g., 16 mm or more, 17 mm or more, 18 mm or more, 19 mm or more, 20 mm or more, 21 mm or more, 22 mm or more, 23 mm or more, 24 mm or more, 25 mm or more, 26 mm or more, 27 mm or more, 28 mm or more, 29 mm or more, 30 mm or more, 31 mm or more, 32 mm or more, 33 mm or more, 34 mm or more, 35 mm or more, 36 mm or more, 37 mm or more, 38 mm or more, 39 mm or more, 40 mm or more, 45 mm or more, 50 mm or more, 55 mm or more, 60 mm or more, 65 mm or more, or 70 mm or more). In some examples, the housing 306 can have an average characteristic dimension of 75 mm or less (e.g., 70 mm or less, 65 mm or less, 60 mm or less, 55 mm or less, 50 mm or less, 45 mm or less, 40 mm or less, 39 mm or less, 38 mm or less, 37 mm or less, 36 mm or less, 35 mm or less, 34 mm or less, 33 mm or less, 32 mm or less, 31 mm or less, 30 mm or less, 29 mm or less, 28 mm or less, 27 mm or less, 26 mm or less, 25 mm or less, 24 mm or less, 23 mm or less, 22 mm or less, 21 mm or less, 20 mm or less, 19 mm or less, 18 mm or less, 17 mm or less, or 16 mm or less). The average characteristic dimension of the housing 306 can range from any of the minimum values described above to any of the maximum values described above. For example, the housing 306 can have an average characteristic dimension of from 15 mm to 75 mm (e.g., from 15 mm to 45 mm, from 45 mm to 75 mm, from 15 mm to 35 mm, from 35 mm to 55 mm, from 55 mm to 75 mm, from 15 mm to 70 mm, from 20 mm to 75 mm, from 20 mm to 70 mm, from 20 mm to 40 mm, or from 30 mm to 36 mm).

The interior cavity 308 can have any shape, such as a regular shape, an irregular shape, an isotropic shape, or an anisotropic shape. For example, the interior cavity 308 can be a polyhedron (e.g., a platonic solid, a prism, a pyramid), a cylinder, a hemicylinder, an elliptical cylinder, a hemi-elliptical cylinder, a cone, a semicone, etc. In some examples, the interior cavity 308 can have a cylindrical shape.

The interior cavity 308 has a longitudinal axis, a proximal end, and a distal end opposite and axially spaced apart from the proximal end. The interior cavity 308 has a length, the length being the dimension along the longitudinal axis from the proximal end to the distal end. The interior cavity 308 can have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the interior cavity 308 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the interior cavity 308 can range from any of the minimum values described above to any of the maximum values described above. For example, the interior cavity 308 can have an length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 30 mm to 150 mm, from 25 mm to 145 mm, from 30 mm to 145 mm, from 30 mm to 75 mm, or from 60 mm to 70 mm).

The interior cavity 308 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the interior cavity 308 can be substantially circular, ovate, ovoid, elliptic, triangular, rectangular, polygonal, etc.

The interior cavity 308 can have an average characteristic dimension. The term “characteristic dimension,” as used herein refers to the largest straight line distance between two points in the plane of the cross-sectional shape of the interior cavity 308. “Average characteristic dimension” and “mean characteristic dimension” are used interchangeably herein, and generally refer to the statistical mean characteristic dimension. For example, for a cylindrical interior cavity 308, the cross-sectional shape can be substantially circular and the average characteristic dimension can refer to the average diameter.

For example, the interior cavity 308 can have an average characteristic dimension of 5 mm or more (e.g., 6 mm or more, 7 mm or more, 8 mm or more, 9 mm or more, 10 mm or more, 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the interior cavity 308 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, or 6 mm or less). The average characteristic dimension of the interior cavity 308 can range from any of the minimum values described above to any of the maximum values described above. For example, the interior cavity 308 can have an average characteristic dimension of from 5 mm to 50 mm (e.g., from 5 mm to 30 mm, from 30 mm to 50 mm, from 5 mm to 20 mm, from 20 mm to 30 mm, from 30 mm to 40 mm, from 40 mm to 50 mm, from 10 mm to 50 mm, from 5 mm to 48 mm, from 10 mm to 48 mm, from 10 mm to 25 mm, or from 15 mm to 23 mm).

The first compartment 312 can have any shape, such as a regular shape, an irregular shape, an isotropic shape, or an anisotropic shape. For example, the first compartment 312 can be a polyhedron (e.g., a platonic solid, a prism, a pyramid), a cylinder, a hemicylinder, an elliptical cylinder, a hemi-elliptical cylinder, a cone, a semicone, etc. In some examples, the first compartment 312 can have a cylindrical shape.

In some examples, the first compartment 312 is disposed coaxially with the filter 310.

The first compartment 312 has a longitudinal axis, with the proximal end 316 being opposite and axially spaced apart from the distal end 318. The first compartment 312 has a length, the length being the dimension along the longitudinal axis from the proximal end 316 to the distal end 318. The first compartment 312 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the first compartment 312 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the first compartment 312 can range from any of the minimum values described above to any of the maximum values described above. For example, the first compartment 312 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 60 mm to 75 mm, or from 60 mm to 70 mm).

The first compartment 312 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the first compartment 312 can be substantially circular, ovate, ovoid, elliptic, triangular, rectangular, polygonal, etc.

The first compartment 312 can have an average characteristic dimension. The term “characteristic dimension,” as used herein refers to the largest straight line distance between two points in the plane of the cross-sectional shape of the first compartment 312. “Average characteristic dimension” and “mean characteristic dimension” are used interchangeably herein, and generally refer to the statistical mean characteristic dimension. For example, for a cylindrical first compartment 312, the cross-sectional shape can be substantially circular and the average characteristic dimension can refer to the average diameter.

For example, the first compartment 312 can have an average characteristic dimension of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the first compartment 312 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The average characteristic dimension of the first compartment 312 can range from any of the minimum values described above to any of the maximum values described above. For example, the first compartment 312 can have an average characteristic dimension of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm).

The filter 310 can comprise any suitable material, such as those known in the art. In some examples, the filter 310 can comprise a polymer, a composite material, a metal, or a combination thereof. In some examples, the filter 310 can comprise a polymer, such as nylon, polyester, polytetrafluoroethylene (PTFE), an acetal resin (e.g., Delrin®), polyether ether ketone (PEEK), derivatives thereof, or combinations thereof. In some examples, the filter 310 can comprise a metal, such as titanium, stainless steel, derivatives thereof, or combinations thereof. The filter 310 can, in some example, be different than the filter 210. For example, the filter 310 can be selected to separate the desired component from the effluent.

In some examples, the filter 310 comprises a mesh filter. The mesh filter 310 can comprise a mesh with a plurality of openings. The plurality of openings of the filter 310 can, for example, have an average size of 5 micrometers (microns, μm) or more (e.g., 6 μm or more, 7 μm or more, 8 μm or more, 9 μm or more, 10 μm or more, 11 μm or more, 12 μm or more, 13 μm or more, 14 μm or more, 15 μm or more, 16 μm or more, 17 μm or more, 18 μm or more, or 19 μm or more). In some examples, the plurality of openings of the filter 310 can have an average size of 20 μm or less (e.g., 19 μm or less, 18 μm or less, 17 μm or less, 16 μm or less, 15 μm or less, 14 μm or less, 13 μm or less, 12 μm or less, 11 μm or less, 10 μm or less, 9 μm or less, 8 μm or less, 7 μm or less, or 6 μm or less). The average size of the plurality of openings of the filter 310 can range from any of the minimum values described above to any of the maximum values described above. For example, the plurality of openings of the filter 310 can have an average size of from 5 μm to 20 μm (e.g., from 5 μm to 12.5 μm, from 12.5 μm to 20 μm, from 5 μm to 10 μm, from 10 μm to 15 μm, from 15 μm to 20 μm, from 6 μm to 20 μm, from 5 μm to 19 μm, or from 6 μm to 19 μm). The average size of the plurality of openings can, for example, be selected to optimize cleaning and concentration of the desired component.

The filter 310 can have any shape, such as a regular shape, an irregular shape, an isotropic shape, or an anisotropic shape. For example, the filter 310 can be a polyhedron (e.g., a platonic solid, a prism, a pyramid), a cylinder, a hemicylinder, an elliptical cylinder, a hemi-elliptical cylinder, a cone, a semicone, etc. In some examples, the filter 310 can have a cylindrical shape.

The filter 310 has a longitudinal axis, a proximal end, and a distal end opposite and axially spaced apart from the proximal end. The filter 310 has a length, the length being the dimension along the longitudinal axis from the proximal end to the distal end.

The filter 310 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the filter 310 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the filter 310 can range from any of the minimum values described above to any of the maximum values described above. For example, the filter 310 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 40 mm to 60 mm, or from 48 mm to 56 mm). For example, the length of the filter 310 can be selected in view of the length of the interior cavity 308.

The filter 310 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the filter 310 can be substantially circular, ovate, ovoid, elliptic, triangular, rectangular, polygonal, etc.

The filter 310 can have an average characteristic dimension. The term “characteristic dimension,” as used herein refers to the largest straight line distance between two points in the plane of the cross-sectional shape of the filter 310. “Average characteristic dimension” and “mean characteristic dimension” are used interchangeably herein, and generally refer to the statistical mean characteristic dimension. For example, for a cylindrical filter 310, the cross-sectional shape can be substantially circular and the average characteristic dimension can refer to the average diameter.

For example, the filter 310 can have an average characteristic dimension of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the filter 310 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The average characteristic dimension of the filter 310 can range from any of the minimum values described above to any of the maximum values described above. For example, the filter 310 can have an average characteristic dimension of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm). The average characteristic dimension of the filter 310 can, for example, be selected in view of the average characteristic dimension of the interior cavity 308.

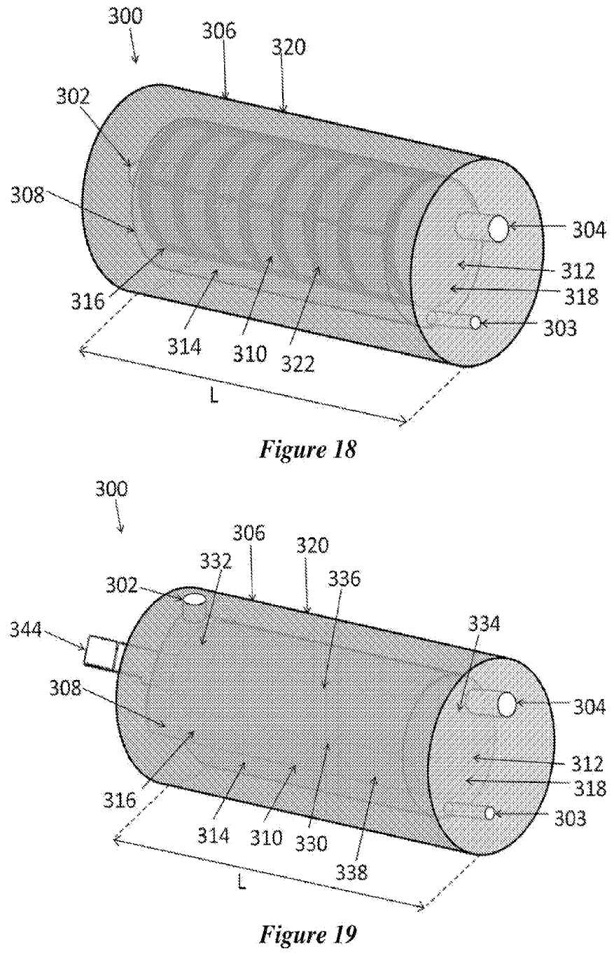

Referring now to FIG. 18, in some examples, the effluent separation module 300 further comprises a filter cage 322 disposed circumferentially around and coaxially with the filter 310 within the interior cavity 308. The filter cage 322 can, for example, provide structural support and/or rigidity to the filter 310.

The filter cage 322 can comprise any suitable material, such as those known in the art. In some examples, the filter cage 322 can comprise a polymer, a composite material, a metal, or a combination thereof.

The filter cage 322 can have any shape, such as a regular shape, an irregular shape, an isotropic shape, or an anisotropic shape. For example, the filter cage 322 can be a polyhedron (e.g., a platonic solid, a prism, a pyramid), a cylinder, a hemicylinder, an elliptical cylinder, a hemi-elliptical cylinder, a cone, a semicone, etc. In some examples, the filter cage 322 can have a cylindrical shape. The shape of the filter case 322 can be selected, for example, in view of the shape of the filter 310 and/or the interior cavity 308.

The filter cage 322 has a longitudinal axis, a proximal end, and a distal end opposite and axially spaced apart from the proximal end. The filter cage 322 has a length, the length being the dimension along the longitudinal axis from the proximal end to the distal end.

The filter cage 322 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the filter cage 322 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the filter can range from any of the minimum values described above to any of the maximum values described above. For example, the filter cage 322 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 40 mm to 60 mm, or from 48 mm to 56 mm). The length of the filter case 322 can be selected, for example, in view of the length of the filter 310 and/or the interior cavity 308.

The filter cage 322 can have a cross-sectional shape in a plane perpendicular to the longitudinal axis, wherein the cross-sectional shape can be any shape, such as a regular shape, an irregular shape, an isotropic shape, or an anisotropic shape. In some examples, the cross-sectional shape of the filter cage 322 can be substantially circular, ovate, ovoid, elliptic, triangular, rectangular, polygonal, etc.

The filter cage 322 can have an average characteristic dimension. The term “characteristic dimension,” as used herein refers to the largest straight line distance between two points in the plane of the cross-sectional shape of the filter cage 322. “Average characteristic dimension” and “mean characteristic dimension” are used interchangeably herein, and generally refer to the statistical mean characteristic dimension. For example, for a cylindrical filter cage 322, the cross-sectional shape can be substantially circular and the average characteristic dimension can refer to the average diameter.

For example, the filter cage 322 can have an average characteristic dimension of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the filter cage 322 has an average characteristic dimension of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The average characteristic dimension of the filter cage 322 can range from any of the minimum values described above to any of the maximum values described above. For example, the filter cage 322 can have an average characteristic dimension of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm). The average characteristic dimension of the filter cage 322 can be selected, for example, in view of the average characteristic dimension of the filter 310 and/or the interior cavity 308.

Referring now to FIG. 19, in some examples, the effluent separation module 300 further comprises: a rotary implement 330 having longitudinal axis, a proximal end 332, and a distal end 334 axially spaced apart from the proximal end, the rotary implement 330 comprising a central shaft 336 and a blade 338 extending from the central shaft 336; wherein the filter is configured to be disposed circumferentially around and coaxially with the rotary implement 330 within the interior cavity 308, such that the rotary implement 330 is configured to be rotatably disposed within the first compartment 312 with the proximal end 332 of the rotary implement 330 disposed towards the proximal end 316 of the first compartment 312 and the distal end 334 of the rotary implement 330 being disposed towards the distal end 318 of the first compartment 312; and wherein the rotary implement 330 is configured to agitate the effluent within the first compartment 312 via rotation of the rotary implement 330.

The rotary implement 330 can comprise any suitable material, such as those known in the art. In some examples, the rotary implement 330 can comprise a polymer, a composite material, a metal, or a combination thereof. In some examples, the rotary implement 330 can comprise polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof. In some examples, the rotary implement 330 can comprise polycarbonate. In some examples, the rotary implement 330 can comprise a metal, such as titanium, stainless steel, derivatives thereof, or combinations thereof.

The rotary implement 330 has a length, the length being the dimension along the longitudinal axis from the proximal end to the distal end. The rotary implement 330 can, for example, have a length of 25 mm or more (e.g., 30 mm or more, 35 mm or more, 40 mm or more, 45 mm or more, 50 mm or more, 55 mm or more, 60 mm or more, 65 mm or more, 70 mm or more, 75 mm or more, 80 mm or more, 85 mm or more, 90 mm or more, 95 mm or more, 100 mm or more, 105 mm or more, 110 mm or more, 115 mm or more, 120 mm or more, 125 mm or more, 130 mm or more, 135 mm or more, 140 mm or more, or 145 mm or more). In some examples, the rotary implement 330 can have a length of 150 mm or less (e.g., 145 mm or less, 140 mm or less, 135 mm or less, 130 mm or less, 125 mm or less, 120 mm or less, 115 mm or less, 110 mm or less, 105 mm or less, 100 mm or less, 95 mm or less, 90 mm or less, 85 mm or less, 80 mm or less, 75 mm or less, 70 mm or less, 65 mm or less, 60 mm or less, 55 mm or less, 50 mm or less, 45 mm or less, 40 mm or less, 35 mm or less, or 30 mm or less). The length of the rotary implement 230 can range from any of the minimum values described above to any of the maximum values described above. For example, the rotary implement 230 can have a length of from 25 mm to 150 mm (e.g., from 25 mm to 90 mm, from 90 mm to 150 mm, from 25 mm to 50 mm, from 50 mm to 75 mm, from 75 mm to 100 mm, from 100 mm to 125 mm, from 125 mm to 150 mm, from 25 mm to 140 mm, from 35 mm to 150 mm, from 35 mm to 140 mm, from 40 mm to 60 mm, or from 48 mm to 56 mm). The length of the rotary implement 330 can be selected, for example, in view of the length of the filter 310 and/or the interior cavity 308.

For example, the central shaft 336 of the rotary implement 330 can have a diameter of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the central shaft 336 of the rotary implement 330 can have a diameter of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The diameter of the central shaft 336 of the rotary implement 330 can range from any of the minimum values described above to any of the maximum values described above. For example, the central shaft 336 of the rotary implement 330 can have a diameter of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 12 mm to 19 mm).

In some examples, the blade 338 of the rotary implement 330 has an edge and the edge and the filter 310 are radially spaced apart from each other by a distance of 0 micrometers (microns, μm) or more (e.g., 1 μm or more, 2 μm or more, 3 μm or more, 4 μm or more, 5 μm or more, 10 μm or more, 15 μm or more, 20 μm or more, 25 μm or more, 30 μm or more, 40 μm or more, 50 μm or more, 75 μm or more, 100 μm or more, 125 μm or more, 150 μm or more, 175 μm or more, 200 μm or more, 250 μm or more, 300 μm or more, 350 μm or more, 400 μm or more, 450 μm or more, 500 μm or more, 600 μm or more, 700 μm or more, 800 μm or more, 900 μm or more, 1 millimeters (mm) or more, 1.25 mm or more, 1.5 mm or more, or 1.75 mm or more). In some examples, the edge and the filter 310 are radially spaced apart from each other by a distance of 2 mm or less (e.g., 1.75 mm or less, 1.5 mm or less, 1.25 mm or less, 1 mm or less, 900 μm or less, 800 μm or less, 700 μm or less, 600 μm or less, 500 μm or less, 450 μm or less, 400 μm or less, 350 μm or less, 300 μm or less, 250 μm or less, 200 μm or less, 175 μm or less, 150 μm or less, 125 μm or less, 100 μm or less, 75 μm or less, 50 μm or less, 40 μm or less, 30 μm or less, 25 μm or less, 20 μm or less, 15 μm or less, 10 μm or less, 5 μm or less, 4 μm or less, 3 μm or less, 2 μm or less, or 1 μm or less). The distance that the edge and the filter 310 are radially spaced apart from each other can range from any of the minimum values described above to any of the maximum values described above. For example, the edge and the filter 310 can be radially spaced apart from each other by a distance of from 0 μm to 2 mm (e.g., from 0 μm to 100 μm, from 100 μm to 2 mm, from 0 μm to 10 μm, from 10 μm to 100 μm, from 100 μm to 1 mm, from 1 mm to 2 mm, from 0 μm to 1.75 mm, from 1 μm to 2 mm, or from 1 μm to 1.75 mm). The distance can, for example, be selected to optimize the cleaning and concentration of the desired component and/or to minimize damage to the desired component.

The rotary implement 330 has an outer diameter as measured to the edge of the blade 338 (e.g., edge to edge for a helical blade 338). For example, the rotary implement 330 can have an outer diameter of 5 mm or more (e.g., 6 mm or more, 7 mm or more, 8 mm or more, 9 mm or more, 10 mm or more, 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the rotary implement 330 can have an outer diameter of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, or 6 mm or less). The outer diameter of the rotary implement 330 can range from any of the minimum values described above to any of the maximum values described above. For example, the rotary implement 330 can have an outer diameter of from 5 mm to 50 mm (e.g., from 5 mm to 30 mm, from 30 mm to 50 mm, from 5 mm to 20 mm, from 20 mm to 30 mm, from 30 mm to 40 mm, from 40 mm to 50 mm, from 10 mm to 50 mm, from 5 mm to 48 mm, from 10 mm to 48 mm, from 10 mm to 25 mm, or from 10 mm to 15 mm).

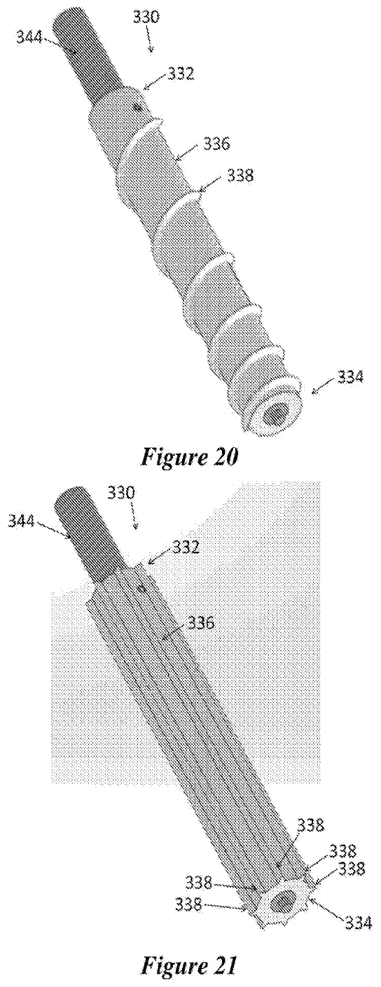

The rotary implement 330 can have any suitable configuration. Example rotary implements 330 are shown in FIG. 20 to FIG. 24.

In some examples, the blade 338 is helically disposed about the central shaft 236.

Referring now to FIG. 20, in some examples, the blade 338 is helically disposed about the central shaft 336 with a variable pitch.

Referring now to FIG. 21, in some examples, the blade 338 comprises a plurality of blades 338, each of the blades 338 extending radially from the central shaft 336 and being disposed circumferentially about the central shaft 336.

In some examples, the rotary implement 330 comprises a first section 340 and a second section 342, the first section 340 extending along a first portion of the central shaft 336 towards the proximal end 332 of the rotary implement 330 and the second section 342 being adjacent the first section and extending along a second portion of the central shaft 336, wherein the first section 340 has a first blade 338A extending from the central shaft 336 and the second section 342 has a second blade 338B extending from the central shaft 336.

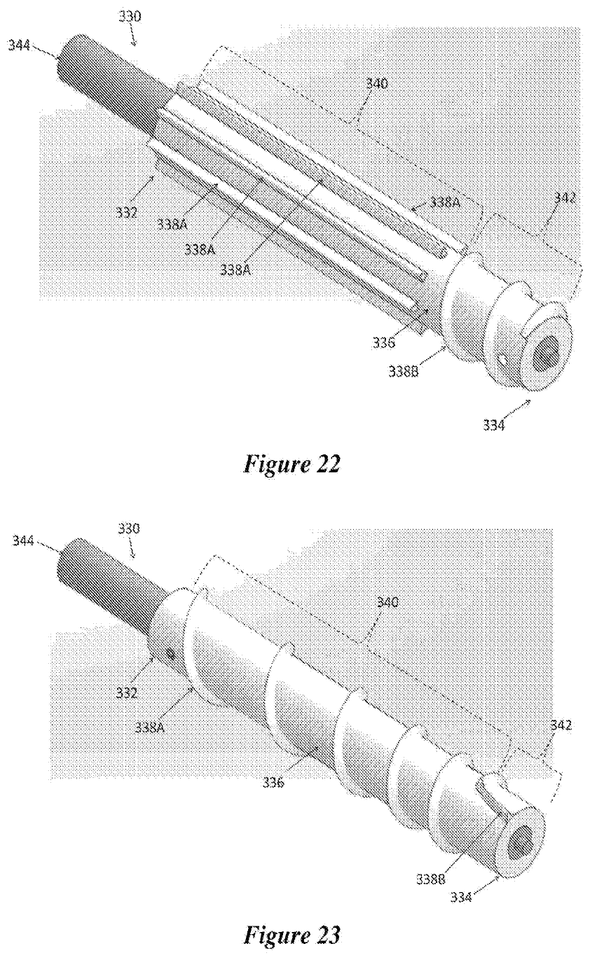

Referring now to FIG. 22, in some examples, the second blade 338B is helically disposed about the central shaft 336 and the first blade 338A comprises a plurality of first blades 338A, each of the first blades 338A extending radially from the central shaft 336 and being disposed circumferentially about the central shaft 336.

Referring now to FIG. 23, in some examples, the first blade 338A is helically disposed about the central shaft 336 and the second blade 338B is a single blade extending radially from the central shaft 336, wherein the first section 340 borders the second section 342 and the first blade 338A is joined to the second blade 338B at the border between the first section 340 and the second section 342. In some examples, the first blade 338A is helically disposed about the central shaft 336 with a variable pitch.

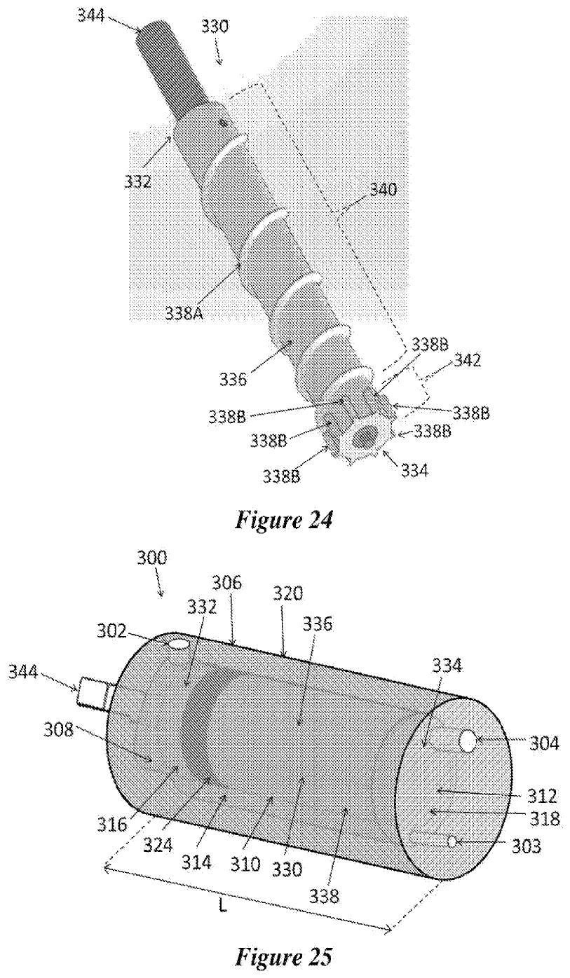

Referring now to FIG. 24, in some examples, the first blade 338A is helically disposed about the central shaft 336 and the second blade 338B comprises a plurality of second blades 338B, each of the second blades 338B extending radially from the central shaft 336 and being disposed circumferentially about the central shaft 336.

In some examples, the effluent separation module 300 further comprises a rod 344 and wherein the central shaft 336 of the rotary implement 330 is disposed circumferentially around and coaxially with the rod 344.

The rod 344 can comprise any suitable material, such as those known in the art. In some examples, the rod 344 can comprise a polymer, a composite material, a metal, or a combination thereof. In some examples, the rod 344 can comprise a metal, such as titanium, stainless steel, derivatives thereof, or combinations thereof.

Referring now to FIG. 25, in some examples, the effluent separation module 300 further comprises a sliding ring 324, the sliding ring 324 being slidably disposed between the rotary implement 330 and an inner surface of the filter 310, such that the sliding ring 324 is disposed circumferentially around and coaxially with the rotary implement 330 and the inner surface of the filter 310 is disposed circumferentially around and coaxially with the sliding ring 324. The sliding ring 324 can be configured, for example, to slide axially to clear the inner surface of the filter 310.

The sliding ring 344 can be configured to be coupled to a means for axially sliding the sliding ring 344. The means for sliding the sliding ring 344 can be a manual means or an automated means. Examples of suitable means for sliding the sliding ring 344 include, but are not limited to, handles, actuators, magnetic coupling, and combinations thereof. In some examples, the means for sliding the sliding ring 344 can comprise an actuator coupled to the sliding ring 324. In some examples, the kit further comprises the means for sliding the sliding ring 344.

The sliding ring 324 can comprise any suitable material, such as those known in the art. In some examples, the sliding ring 324 can comprise a polymer, a composite material, a metal, or a combination thereof. In some examples, the sliding ring 324 can comprise a polymer, such as polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof. In some examples, the sliding ring 324 can comprise a magnetic material.

The sliding ring 324 has a longitudinal axis, a proximal end, and a distal end opposite and axially spaced apart from the proximal end. The sliding ring 324 has a length, the length being the dimension along the longitudinal axis from the proximal end to the distal end. The sliding ring 324 can have a length of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the sliding ring 324 can have a length of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The length of the sliding ring 324 can range from any of the minimum values described above to any of the maximum values described above. For example, the sliding ring 324 can have a length of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 10 mm to 15 mm).

The sliding ring 324 can, for example, have an outer diameter of 10 mm or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 30 mm or more, 35 mm or more, 40 mm or more, or 45 mm or more). In some examples, the sliding ring 324 can have an outer diameter of 50 mm or less (e.g., 45 mm or less, 40 mm or less, 35 mm or less, 30 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The outer diameter of the sliding ring 324 can range from any of the minimum values described above to any of the maximum values described above. For example, the sliding ring 324 can have an outer diameter of from 10 mm to 50 mm (e.g., from 10 mm to 30 mm, from 30 mm to 50 mm, from 10 mm to 20 mm, from 20 mm to 30 mm, from 30 mm to 40 mm, from 40 mm to 50 mm, from 12 mm to 50 mm, from 10 mm to 48 mm, from 12 mm to 48 mm, from 10 mm to 25 mm, or from 10 mm to 15 mm).

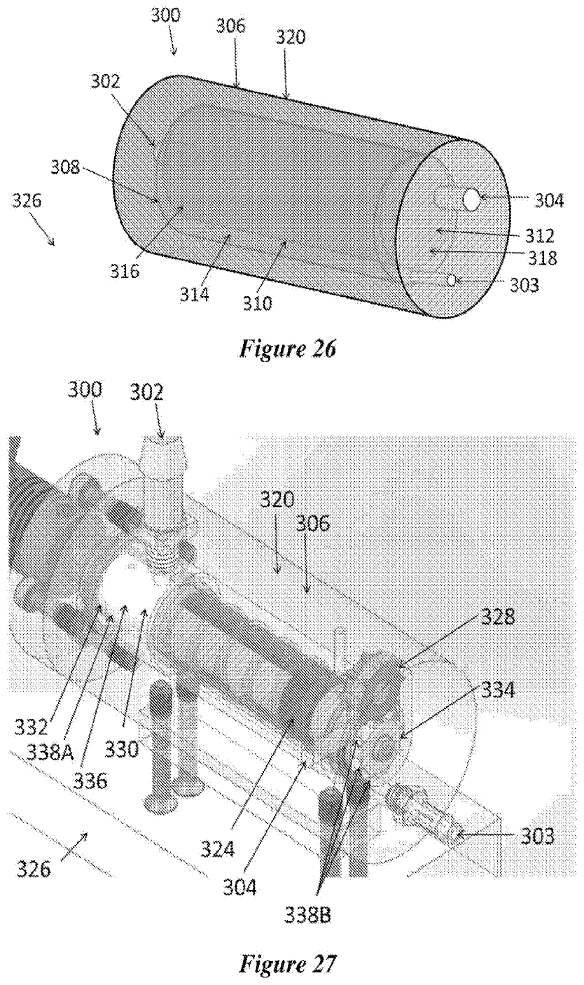

Referring now to FIG. 26, in some examples, the effluent separation module 300 is disposed on a platform 326. The effluent separation module 300 can, for example, be attached to the platform 326 using adhesives, bolts, screws, clips, or any other mechanical or chemical fastener known in the art.

The platform 326 can comprise any suitable material, such as those known in the art. In some examples, the platform 326 can comprise a polymer, a composite material, a metal, or a combination thereof. In some examples, the platform 326 can comprise a polymer, such as polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof.

In some examples, the rotary implement 330 is configured to be coupled to a means for rotating 328 the rotary implement 330, wherein the means for rotating 328 the rotary implement 330 is configured to rotate the rotary implement 330 within the first compartment 312.

The means for rotating 328 the rotary implement 330 can comprise any suitable means, such as those known in the art. The means for rotating 328 the rotary implement can be a manual means or an automated means. Examples of suitable means for rotating 328 the rotary implement include, but are not limited to, cranks, motors, gears, and combinations thereof. In some examples, the means for rotating 328 the rotary implement 330 can comprise a motor, such as variable speed motor. In some examples, the kit 100 further comprises a means for rotating 328 the rotary implement 330 of the effluent separation module 300.

For example, the means for rotating 328 the rotary implement 330 can comprise a variable speed motor, such as a gear motor with a controllable speed. For example, the motor 328 can have a speed of 2 RPM or more (e.g., 3 RPM or more, 4 RPM or more, 5 RPM or more, 6 RPM or more, 7 RPM or more, 8 RPM or more, 9 RPM or more, 10 RPM or more, 11 RPM or more, 12 RPM or more, 13 RPM or more, 14 RPM or more, 15 RPM or more, 16 RPM or more, 17 RPM or more, 18 RPM or more, 19 RPM or more, 20 RPM or more, 21 RPM or more, 22 RPM or more, 23 RPM or more, or 24 RPM or more). In some examples, the motor 328 can have a speed of 25 RPM or less (e.g., 24 RPM or less, 23 RPM or less, 22 RPM or less, 21 RPM or less, 20 RPM or less, 19 RPM or less, 18 RPM or less, 17 RPM or less, 16 RPM or less, 15 RPM or less, 14 RPM or less, 13 RPM or less, 12 RPM or less, 11 RPM or less, 10 RPM or less, 9 RPM or less, 8 RPM or less, 7 RPM or less, 6 RPM or less, 5 RPM or less, 4 RPM or less, or 3 RPM or less). The speed of the motor 228 can range from any of the minimum values described above to any of the maximum values described above. For example, the motor 328 can have a speed of from 2 RPM to 25 RPM (e.g., from 2 to 13 RPM, from 13 to 25 RPM, from 2 to 5 RPM, from 5 to 10 RPM, from 10 to 15 RPM, from 15 to 20 RPM, from 20 to 25 RPM, from 3 to 25 RPM, from 4 to 25 RPM, from 5 to 25 RPM, from 6 to 25 RPM, from 8 to 25 RPM, from to 25 RPM, from 15 to 25 RPM, from 2 to 24 RPM, from 2 to 23 RPM, from 2 to 22 RPM, from 2 to 21 RPM, from 2 to 20 RPM, from 2 to 18 RPM, from 2 to 15 RPM, from 2 to 10 RPM, from 3 to 24 RPM, from 5 to 20 RPM, from 10 to 20 RPM, or from 15 to 20 RPM).

Referring now to FIG. 27, in some examples, the means for rotating 328 the rotary implement 330 of the effluent separation module 300 comprises a gear pump disposed inside the housing 306 and coupled to the rotary implement 330 of the effluent separation module 300.

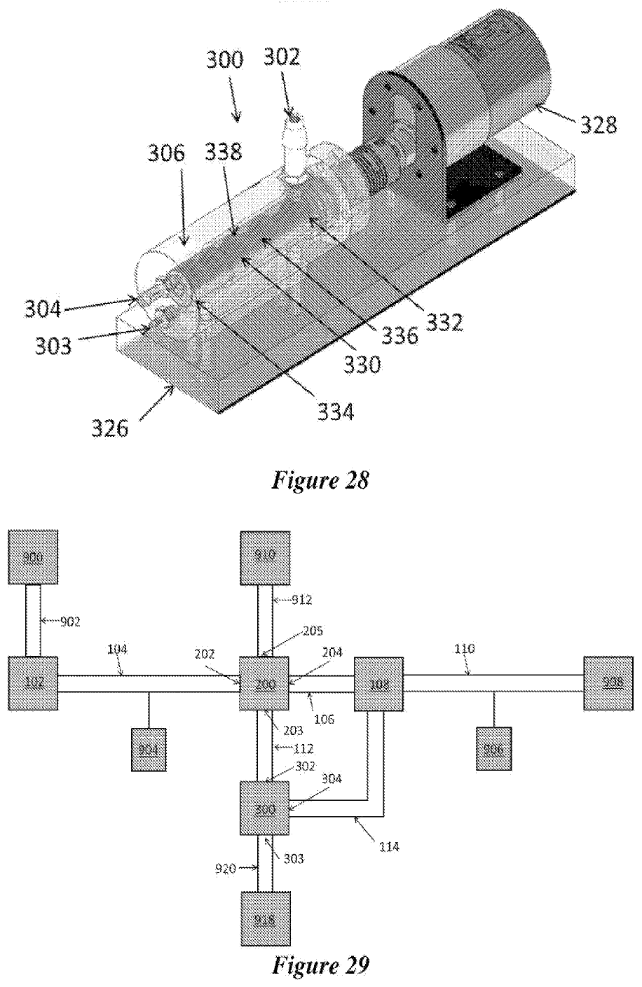

Referring now to FIG. 28, in some examples, the means for rotating 328 the rotary implement 330 of the effluent separation module 300 comprises a motor disposed outside the housing 306 and coupled to the central shaft 336 and/or the rod 344 of the rotary implement 330 of the effluent separation module 300.

In some examples, the effluent separation module 300 further comprises a port 303, wherein the port 330 is defined by the housing 306 and is fluidly connected to the second compartment 314, for example at or near the distal end of the second compartment 314.

The port 303 can, for example, have an inner diameter of 1 mm or more (e.g., 1.5 mm or more, 2 mm or more, 2.5 mm or more, 3 mm or more, 3.5 mm or more, 4 mm or more, 4.5 mm or more, 5 mm or more, 5.5 mm or more, 6 mm or more, 6.5 mm or more, 7 mm or more, 7.5 mm or more, 8 mm or more, 8.5 mm or more, 9 mm or more, 9.5 mm or more, 10 mm or more, 10.5 mm or more, 11 mm or more, or 11.5 mm or more). In some examples, the port 303 can have an inner diameter of 12 mm or less (e.g., 11.5 mm or less, 11 mm or less, 10.5 mm or less, 10 mm or less, 9.5 mm or less, 9 mm or less, 8.5 mm or less, 8 mm or less, 7.5 mm or less, 7 mm or less, 6.5 mm or less, 6 mm or less, 5.5 mm or less, 5 mm or less, 4.5 mm or less, 4 mm or less, 3.5 mm or less, 3 mm or less, 2.5 mm or less, 2 mm or less, or 1.5 mm or less). The inner diameter of the port 303 can range from any of the minimum values described above to any of the maximum values described above. For example, the port 303 can have an inner diameter of from 1 mm to 12 mm (e.g., from 1 mm to 6 mm, from 6 mm to 12 mm, from 1 mm to 4 mm, from 4 mm to 8 mm, from 8 mm to 12 mm, from 2 mm to 12 mm, from 1 mm to 11 mm, or from 2 mm to 11 mm).

Referring now to FIG. 29, in some examples, the port 303 of the effluent separation module 300 is configured to be fluidly connected to a waste receptacle 918, for example via an eighth tube 920, the waste receptacle 918 being configured to receive the waste solution from the port 303 of the effluent separation module 300.

For example, the port 303 of the effluent separation module 300 can be configured to be fluidly connected to the eighth tube 920, wherein the eighth tube 920 is configured to fluidly connect the port 303 of the effluent separation module 300 to an waste receptacle 918. In some examples, the kit 100 further comprises a waste receptacle 918 configured to be fluidly connected to the port 303 of the effluent separation module 300. In some examples, the kit 100 further comprises an eighth tube 920 configured to fluidly connect the port 303 of the effluent separation module 300 to a waste receptacle 918. In some examples, the kit 100 further comprises an eighth tube 920 and a waste receptacle 918, wherein the eighth tube 920 is configured to fluidly connect the port 303 of the effluent separation module 300 to the waste receptacle 918, the waste receptacle 918 being configured to receive the waste solution from the port 303 of the effluent separation module 300.

The waste receptacle 918 can, for example, comprise any suitable container for containing the waste solution, such as those known in the art. In some examples, the waste receptacle 918 can comprise a flexible polymer collection bag, vacutainer, or vacuum canister. In some examples, the waste receptacle 918 can comprise a disposable medical collection bag. In some examples, the waste receptacle 918 containing the waste can be disposable as biohazard waste. The waste receptacle 918 can, for example, comprise any suitable material, such as those known in the art. For example, the waste receptacle 918 can comprise a polymer.

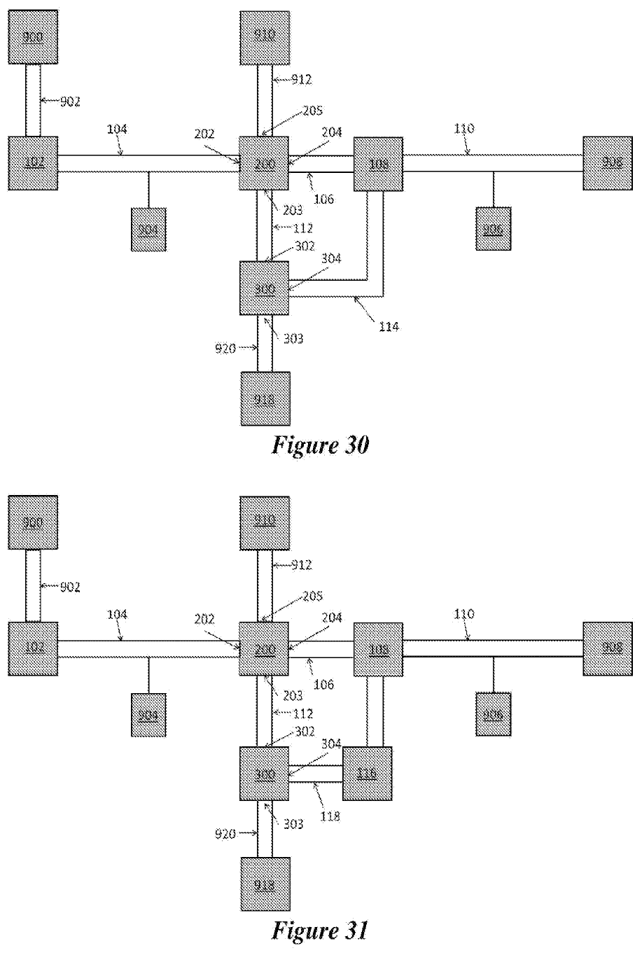

Referring now to FIG. 30, in some examples, the outlet 304 of the effluent separation module 300 is configured to be fluidly connected to the collection reservoir 108, for example via a ninth tube 114, the collection reservoir 108 being configured to receive the desired component from the outlet 304 of the effluent separation module 300. For example, the outlet 304 of the effluent separation module 300 can be configured to be fluidly connected to the ninth tube 114, wherein the ninth tube 114 is configured to fluidly connect the outlet 304 of the effluent separation module 300 to the collection reservoir 108. In some examples, the kit 100 further comprises a ninth tube 114 configured to fluidly connect the outlet 304 of the effluent separation module 300 to the collection reservoir 108.

Referring now to FIG. 31, in some examples, the outlet 304 of the effluent separation module 300 is configured to be fluidly connected to a container 116, for example via a tenth tube 118, the container 116 being configured to receive the desired component from the outlet 304 of the effluent separation module 300.

For example, the outlet 304 of the effluent separation module 300 can be configured to be fluidly connected to the tenth tube 118, wherein the tenth tube 118 is configured to fluidly connect the outlet 304 of the effluent separation module 300 to a container 116. In some examples, the kit 100 further comprises a container 116 configured to be fluidly connected to the outlet 304 of the effluent separation module 300. In some examples, the kit 100 further comprises a tenth tube 118 configured to fluidly connect the outlet 304 of the effluent separation module 300 to a container 116. In some examples, the kit 100 further comprises a tenth tube 118 and a container 116, wherein the tenth tube 118 is configured to fluidly connect the outlet 304 of the effluent separation module 300 to the container 116, the container 116 being configured to receive the desired component from the outlet 304 of the effluent separation module 300.

In some examples, the container 116 can be configured to be fluidly connected to the collection reservoir 108, for example via an eleventh tube 120, the collection reservoir 108 being configured to receive the desired component from container 116. For example, the container 116 can be configured to be fluidly connected to the eleventh tube 120, wherein the eleventh tube 120 is configured to fluidly connect the container 116 to the collection reservoir 108. In some examples, the kit 100 further comprises an eleventh tube 120 configured to fluidly connect the container 116 to the collection reservoir 108.

The container 116 can, for example, comprise any suitable container for containing the desired component, such as those known in the art. In some examples, the container 116 can comprise a sterile and/or flexible medical collection bag. The container 116 can, for example, comprise any suitable material, such as those known in the art. For example, the container 116 can comprise a polymer.

In some examples, the kit 100 further comprises the first tube 902 which is configured to fluidly connect the liposuction cannula 900 to the collection canister 102.

In some examples, the kit 100 further comprises the liposuction cannula 900. The liposuction cannula 900 can, for example, comprise any suitable cannula for liposuction such as those known in the art.

The first tube 902, the second tube 104, the third tube 106, the fourth tube 110, the fifth tube 912 (when present), the sixth tube 916 (when present), the seventh tube 112 (when present), the eighth tube 920 (when present), the ninth tube 114 (when present), the tenth tube 118 (when present), and the eleventh tube 120 (when present) independently can comprise any suitable material, such as those known in the art. For example, the first tube 902, the second tube 104, the third tube 106, the fourth tube 110, the fifth tube 912 (when present), the sixth tube 916 (when present), the seventh tube 112 (when present), the eighth tube 920 (when present), the ninth tube 114 (when present), the tenth tube 118 (when present), and the eleventh tube 120 (when present) independently can comprise a polymer. In some examples, the first tube 902, the second tube 104, the third tube 106, the fourth tube 110, the fifth tube 912 (when present), the sixth tube 916 (when present), the seventh tube 112 (when present), the eighth tube 920 (when present), the ninth tube 114 (when present), the tenth tube 118 (when present), and the eleventh tube 120 (when present) each comprise medical grade tubing.

The first tube 902, the second tube 104, the third tube 106, the fourth tube 110, the fifth tube 912 (when present), the sixth tube 916 (when present), the seventh tube 112 (when present), the eighth tube 920 (when present), the ninth tube 114 (when present), the tenth tube 118 (when present), and the eleventh tube 120 (when present) can each independently have an inner diameter of 2 mm or more (e.g., 3 mm or more, 4 mm or more, 5 mm or more, 6 mm or more, 7 mm or more, 8 mm or more, 9 mm or more, 10 mm or more, 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, or 24 mm or more). In some examples, The first tube 902, the second tube 104, the third tube 106, the fourth tube 110, the fifth tube 912 (when present), the sixth tube 916 (when present), the seventh tube 112 (when present), the eighth tube 920 (when present), the ninth tube 114 (when present), the tenth tube 118 (when present), and the eleventh tube 120 (when present) can each independently have an inner diameter of 25 mm or less (e.g., 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, 6 mm or less, 5 mm or less, 4 mm or less, or 3 mm or less). The inner diameter of the first tube 902, the second tube 104, the third tube 106, the fourth tube 110, the fifth tube 912 (when present), the sixth tube 916 (when present), the seventh tube 112 (when present), the eighth tube 920 (when present), the ninth tube 114 (when present), the tenth tube 118 (when present), and the eleventh tube 120 (when present) can independently range from any of the minimum values described above to any of the maximum values described above. For example, the first tube 902, the second tube 104, the third tube 106, the fourth tube 110, the fifth tube 912 (when present), the sixth tube 916 (when present), the seventh tube 112 (when present), the eighth tube 920 (when present), the ninth tube 114 (when present), the tenth tube 118 (when present), and the eleventh tube 120 (when present) can each independently have an inner diameter of from 2 mm to 25 mm (e.g., from 2 mm to 14 mm, from 14 mm to 25 mm, from 2 mm to 5 mm, from 5 mm to 10 mm, from 10 mm to 15 mm, from 15 mm to 20 mm, from 20 mm to 25 mm, from 4 mm to 25 mm, from 2 mm to 23 mm, from 4 mm to 23 mm, or from 2 mm to 10 mm).

The first pump 904 and the second pump 906 can independently comprise any suitable pump, such as those known in the art. In some examples, the first pump 904 and the second pump 906 can each independently be a peristaltic pump (e.g., a roller pump), a diaphragm pump, or a gear pump. In some examples, the first pump 904 and the second pump 906 are each independently a roller pump. In some examples, the kit further comprises the first pump 904, the second pump 906, or a combination thereof.

In some examples, the first pump 904 can comprise a roller pump, for example with an adjustable flow rate. For example, the flow rate of the first roller pump 904 can be 20 milliliters per minute (mL/min) or more (e.g., 25 mL/min or more, 30 mL/min or more, 35 mL/min or more, 40 mL/min or more, 45 mL/min or more, 50 mL/min or more, 55 mL/min or more, 60 mL/min or more, 65 mL/min or more, 70 mL/min or more, 75 mL/min or more, 80 mL/min or more, 85 mL/min or more, 90 mL/min or more, 95 mL/min or more, 100 mL/min or more, 105 mL/min or more, 110 mL/min or more, 115 mL/min or more, 120 mL/min or more, 125 mL/min or more, 130 mL/min or more, 135 mL/min or more, 140 mL/min or more, 145 mL/min or more, 150 mL/min or more, 155 mL/min or more, 160 mL/min or more, 165 mL/min or more, 170 mL/min or more, 175 mL/min or more, 180 mL/min or more, 185 mL/min or more, 190 mL/min or more, or 195 mL/min or more). In some examples, the flow rate of the first roller pump can be 200 mL/min or less (e.g., 195 mL/min or less, 190 mL/min or less, 185 mL/min or less, 180 mL/min or less, 175 mL/min or less, 170 mL/min or less, 165 mL/min or less, 160 mL/min or less, 155 mL/min or less, 150 mL/min or less, 145 mL/min or less, 140 mL/min or less, 135 mL/min or less, 130 mL/min or less, 125 mL/min or less, 120 mL/min or less, 115 mL/min or less, 110 mL/min or less, 105 mL/min or less, 100 mL/min or less, 95 mL/min or less, 90 mL/min or less, 85 mL/min or less, 80 mL/min or less, 75 mL/min or less, 70 mL/min or less, 65 mL/min or less, 60 mL/min or less, 55 mL/min or less, 50 mL/min or less, 45 mL/min or less, 40 mL/min or less, 35 mL/min or less, 30 mL/min or less, or 25 mL/min or less). The flow rate of the first roller pump 904 can range from any of the minimum values described above to any of the maximum values described above. For example, the first roller pump 904 can have a flow rate of from 20 mL/min to 200 mL/min (e.g., from 20 to 110 mL/min, from 110 to 200 mL/min, from 20 to 80 mL/min, from 80 to 120 mL/min, from 120 to 160 mL/min, from 160 to 200 mL/min, from 20 to 40 mL/min, from 40 to 80 mL/min, from 80 to 100 mL/min, from 100 to 120 mL/min, from 120 to 140 mL/min, from 140 to 160 mL/min, from 160 to 180 mL/min, from 180 to 200 mL/min, from 20 to 190 mL/min, from 20 to 180 mL/min, from 20 to 160 mL/min, from 20 to 140 mL/min, from 20 to 120 mL/min, from 20 to 100 mL/min, from 30 to 200 mL/min, from 40 to 200 mL/min, from 50 to 200 mL/min, from 60 to 200 mL/min, from 70 to 200 mL/min, from 80 to 200 mL/min, from 90 to 200 mL/min, from 100 to 200 mL/min, from 120 to 200 mL/min, from 140 to 200 mL/min, from 30 to 190 mL/min, from 35 to 150 mL/min, from 40 to 100 mL/min, or from 50 to 75 mL/min).

In some examples, the second pump 906 can comprise a roller pump, for example with an adjustable flow rate. For example, the second roller pump 906 can have a flow rate of 10 milliliters per minute (mL/min) or more (e.g., 15 mL/min or more, 20 mL/min or more, 25 mL/min or more, 30 mL/min or more, 35 mL/min or more, 40 mL/min or more, 45 mL/min or more, 50 mL/min or more, 55 mL/min or more, 60 mL/min or more, 65 mL/min or more, 70 mL/min or more, 75 mL/min or more, 80 mL/min or more, 85 mL/min or more, 90 mL/min or more, 95 mL/min or more, 100 mL/min or more, 105 mL/min or more, 110 mL/min or more, 115 mL/min or more, 120 mL/min or more, 125 mL/min or more, 130 mL/min or more, 135 mL/min or more, 140 mL/min or more, 145 mL/min or more, 150 mL/min or more, 155 mL/min or more, 160 mL/min or more, 165 mL/min or more, 170 mL/min or more, or 175 mL/min or more). In some examples, the flow rate of the second roller pump can be 180 mL/min or less (e.g., 175 mL/min or less, 170 mL/min or less, 165 mL/min or less, 160 mL/min or less, 155 mL/min or less, 150 mL/min or less, 145 mL/min or less, 140 mL/min or less, 135 mL/min or less, 130 mL/min or less, 125 mL/min or less, 120 mL/min or less, 115 mL/min or less, 110 mL/min or less, 105 mL/min or less, 100 mL/min or less, 95 mL/min or less, 90 mL/min or less, 85 mL/min or less, 80 mL/min or less, 75 mL/min or less, 70 mL/min or less, 65 mL/min or less, 60 mL/min or less, 55 mL/min or less, 50 mL/min or less, 45 mL/min or less, 40 mL/min or less, 35 mL/min or less, 30 mL/min or less, 25 mL/min or less, 20 mL/min or less, or 15 mL/min or less). The flow rate of the second roller pump 906 can range from any of the minimum values described above to any of the maximum values described above. For example, the second roller pump 906 can have a flow rate of from 10 mL/min to 180 mL/min (e.g., from 10 to 95 mL/min, from 95 to 180 mL/min, from 10 to 50 mL/min, from 50 to 90 mL/min, from 90 to 130 mL/min, from 130 to 180 mL/min, from 10 to 30 mL/min, from 30 to 50 mL/min, from 50 to 70 mL/min, from 70 to 90 mL/min, from 90 to 110 mL/min, from 110 to 130 mL/min, from 130 to 150 mL/min, from 150 to 180 mL/min, from 10 to 170 mL/min, from 10 to 160 mL/min, from 10 to 150 mL/min, from 10 to 140 mL/min, from 10 to 130 mL/min, from 10 to 120 mL/min, from 10 to 110 mL/min, from 10 to 100 mL/min, from 10 to 90 mL/min, from 10 to 80 mL/min, from 10 to 70 mL/min, from 10 to 60 mL/min, from 20 to 180 mL/min, from 30 to 180 mL/min, from 40 to 180 mL/min, from 50 to 180 mL/min, from 60 to 180 mL/min, from 70 to 180 mL/min, from 80 to 180 mL/min, from 90 to 180 mL/min, from 100 to 180 mL/min, from 110 to 180 mL/min, from 120 to 180 mL/min, from 140 to 180 mL/min, from 20 to 160 mL/min, from 30 to 150 mL/min, from 40 to 125 mL/min, from 50 to 100 mL/min, or from 60 to 90 mL/min).

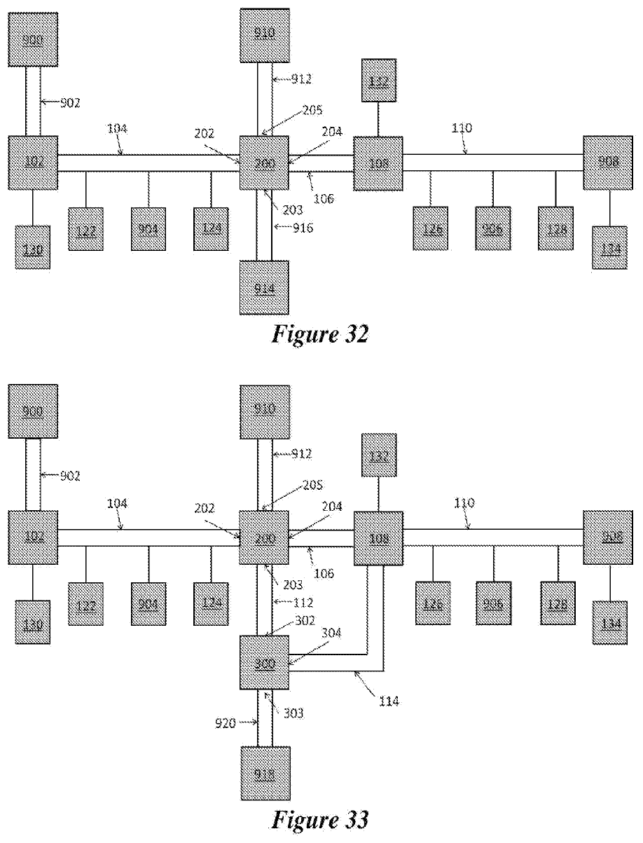

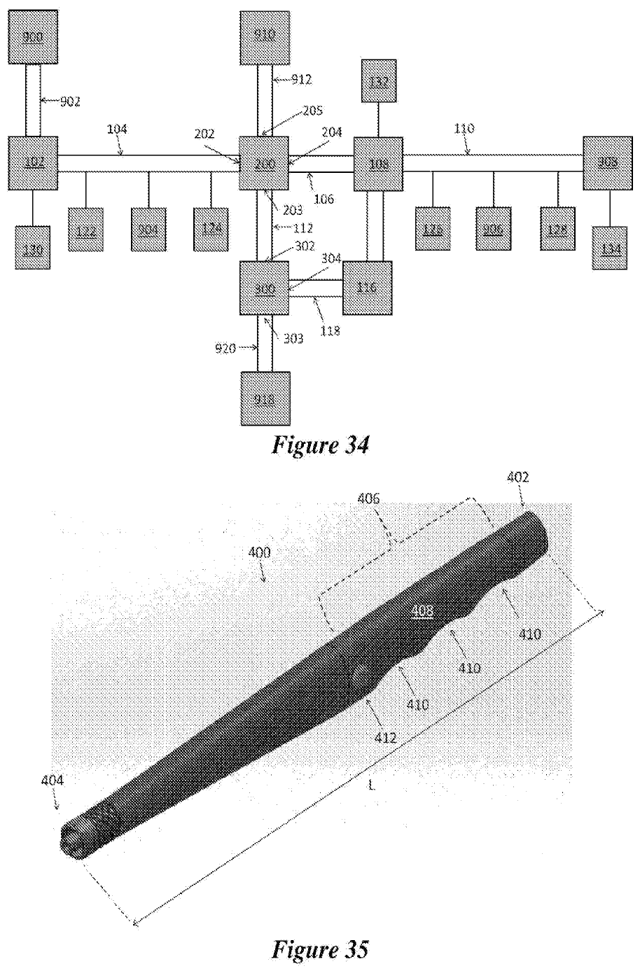

Referring now to FIG. 32-FIG. 34, in some examples, the kit 100 further comprises a first pressure sensor 122 configured to be fluidly connected to the second tube 104 between the first pump 904 and the collection canister 102, wherein the first pressure sensor 122 is configured to detect the pressure between the first pump 904 and the collection canister 102. In some examples, the first pressure sensor 122 is integrally formed with the second tube 104.

In some examples, the kit 100 further comprises a second pressure sensor 124 configured to be fluidly connected to the second tube 104 between the first pump 904 and the adipose separation module 200, wherein the second pressure sensor 124 is configured to detect the pressure between the first pump 904 and the adipose separation module 200. In some examples, the second pressure sensor 124 is integrally formed with the second tube 104.

In some examples, the kit further comprises a third pressure sensor 126 configured to be fluidly connected to the fourth tube 110 between the collection reservoir 108 and the second pump 906, wherein the third pressure sensor 126 is configured to detect the pressure between the collection reservoir 108 and the second pump 906. In some examples, the third pressure sensor 126 is integrally formed with the fourth tube 110.

In some examples, the kit further comprises a fourth pressure sensor 128 configured to be fluidly connected to the fourth tube 110 between the second pump 906 and the injector 908, wherein the fourth pressure sensor 128 is configured to detect the pressure between the second pump 906 and the injector 908. In some examples, the fourth pressure sensor 128 is integrally formed with the fourth tube 110.

In some examples, the kit 100 further comprises a first volume sensor 130 configured to be connected to the collection canister 102 and to detect the volume of the mixture within the collection canister 102. In some examples, the first volume sensor 130 is integrally formed with the collection canister 102.

In some examples, the kit 100 further comprises a second volume sensor 132 configured to be connected to the collection reservoir 108 and to detect the volume of the concentrated adipose tissue within the collection reservoir 108. In some examples, the second volume sensor 132 is integrally formed with the collection reservoir 108.

In some examples, the kit 100 further comprises the injector 908, wherein the injector 908 is configured to receive the concentrated adipose tissue from the collection reservoir 108 via the fourth tube 110 and inject the concentrated adipose tissue into the second anatomical region, for example via an adipose injection cannula. The injector can comprise any suitable injector, such as those known in the art. For example, the injector can comprise a syringe.

In some examples, the injector 908 is configured to be fluidly coupled to an adipose injection cannula. In some examples, the injector 908 further comprises an adipose injection cannula. In some examples, the kit further comprises the adipose injection cannula. The adipose injection cannula can, for example, comprise any suitable cannula for adipose injection such as those known in the art.

In some examples, the injector 908 is configured to be fluidly connected to a fifth pressure sensor 134, wherein the fifth pressure sensor 134 is configured to detect the pressure at which the injector 908 injects the concentrated adipose tissue into the second anatomical region. In some examples, the kit 100 further comprises the fifth pressure sensor 134. In some examples, the injector 908 further comprises the fifth pressure sensor 134. In some examples, the fifth pressure sensor 134 is integrally formed with the injector 908.

Referring now to FIG. 35, in some examples, the injector 908 comprises an ergonomic injection handle 400 has a longitudinal axis, a proximal end 402, and a distal end 404 opposite and axially spaced apart from the proximal end 402. In some examples, the kit further comprises the ergonomic injection handle 400.

In some examples, the distal end 404 of the ergonomic injection handle 400 can be configured to be fluidly connected to an adipose injection cannula. In some examples, the proximal end 402 of the ergonomic injection handle 400 is configured to be fluidly connected to the collection reservoir 108 via the fourth tube 110.

In some examples, the ergonomic injection handle 400 is configured to be coupled to an adipose injection cannula and the fourth tube 110, wherein the fourth tube 110 fluidly connects the collection reservoir 108 to the adipose injection cannula.

The ergonomic injection handle 400 can comprise any suitable material, such as those known in the art. In some examples, the ergonomic injection handle 400 can comprise a polymer, a composite material, a metal, or a combination thereof. In some examples, the ergonomic injection handle 400 can comprise a polymer, such as polycarbonate, acrylonitrile butadiene styrene (ABS), poly(methyl methacrylate) (PMMA), polyvinyl chloride (PVC), an acrylic (e.g., a polyacrylic), nylon, polyester, polytetrafluoroethylene (PTFE), an acetal resin (e.g., Delrin®), polyether ether ketone (PEEK), derivatives thereof, or combinations thereof. In some examples, the ergonomic injection handle 400 can comprise a metal, such as titanium, stainless steel, derivatives thereof, or combinations thereof. In some examples, the ergonomic injection handle 400 can be disposable. In some examples, ergonomic injection handle 400 can be reusable.

In some examples, the ergonomic injection handle 400 has a length, the length being the dimension along the longitudinal axis from the proximal end 402 to the distal end 404 (e.g., “L in FIG. 35). The ergonomic injection handle 400 can, for example, have a length of 10 centimeters (cm) or more (e.g., 11 cm or more, 12 cm or more, 13 cm or more, 14 cm or more, 15 cm or more, 16 cm or more, 17 cm or more, 18 cm or more, or 19 cm or more). In some examples, the ergonomic injection handle 400 can have a length of 20 cm or less (e.g., 19 cm or less, 18 cm or less, 17 cm or less, 16 cm or less, 15 cm or less, 14 cm or less, 13 cm or less, 12 cm or less, or 11 cm or less). The length of the ergonomic injection handle 400 can range from any of the minimum values described above to any of the maximum values described above. For example, the ergonomic injection handle 400 can have a length of from 10 cm to 20 cm (e.g., from 10 cm to 15 cm, from 15 cm to 20 cm, from 10 cm to 12 cm, from 12 cm to 14 cm, from 14 cm to 16 cm, from 16 cm to 18 cm, from 18 cm to 20 cm, from 11 cm to 20 cm, from 10 cm to 19 cm, or from 11 cm to 19 cm).

In some examples, the ergonomic injection handle 400 further comprises a hand grip portion 406 configured to allow a user to grip the ergonomic injection handle 400 comfortably and securely with a hand, the hand grip portion 406 being disposed towards the proximal end 402.

The hand grip portion 406 can, for example, have an average outer diameter of 10 millimeters (mm) or more (e.g., 11 mm or more, 12 mm or more, 13 mm or more, 14 mm or more, 15 mm or more, 16 mm or more, 17 mm or more, 18 mm or more, 19 mm or more, 20 mm or more, 21 mm or more, 22 mm or more, 23 mm or more, 24 mm or more, 25 mm or more, 26 mm or more, 27 mm or more, 28 mm or more, or 29 mm or more). In some examples, the hand grip portion 406 can have an average outer diameter of 30 mm or less (e.g., 29 mm or less, 28 mm or less, 27 mm or less, 26 mm or less, 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, or 11 mm or less). The average outer diameter of the hand grip portion 406 can range from any of the minimum values described above to any of the maximum values described above. For example, the hand grip portion 406 can have an average outer diameter of from 10 mm to 30 mm (e.g., from 10 mm to 20 mm, from 20 mm to 30 mm, from 10 mm to 15 mm, from 15 mm to 20 mm, from 20 mm to 25 mm, from 25 mm to 30 mm, from 12 mm to 30 mm, from 10 mm to 28 mm, or from 12 mm to 28 mm).

In some examples, the hand grip portion 406 comprises a contoured outer surface 408 having a plurality of undulations 410 which are spaced along the hand grip portion 406, said undulations 410 generally conforming to the fingers of a user's hand.

In some examples, the ergonomic injection handle 400 further comprises a button 412 disposed within or proximate the hand grip portion 406, such that the user can easily depress the button 412 with a finger on the hand gripping the hand grip portion 406, wherein the button 412 is configured to inject the concentrated adipose tissue when depressed. In some examples, the button can be configured such that pressing the button causes a single dose of adipose tissue to be injected, a continuous flow of adipose tissue to be injected, or starting/stopping close loop control based on injection or interstitial pressure.

In some examples, the ergonomic injection handle 400 further comprises a feedback indicator, the feedback indicator being configured to provide feedback to a user. The feedback can, for example, comprise haptic feedback (e.g., a vibration motor), auditory feedback (e.g., an auditory alarm), visual feedback, or a combination thereof.

For example, the feedback indicator can comprise one of more visual indicators, such as a colored light emitting diode (LED). In some examples, the feedback indicator can comprise a plurality of LEDs, each LED having a different color, such as a red LED, a yellow LED, a green LED, or a combination thereof. The different colors can provide different feedback to the user.

In some examples, the ergonomic injection handle 400 is configured to be fluidly connected to the fifth pressure sensor 134. In some examples, the ergonomic injection handle 400 further comprises the fifth pressure sensor 134. In some examples, the fifth pressure sensor 134 is integrally formed with the ergonomic injection handle 400.

In some examples, the components of the kit 100 are sterile.

Also disclosed herein are closed-loop adipose transplant systems comprising any of the kits 100 of consumable parts disclosed herein.

For example, also disclosed herein are closed-loop adipose transplant systems comprising a liposuction cannula 900; a first tube 902; a collection canister 102; a second tube 104; a first pump 904; an adipose separation module 200 having an inlet 202 and an outlet 204; a third tube 106; a collection reservoir 108; a fourth tube 110; a second pump 906; and an injector 908. The components of the system together form a continuous, closed fluid pathway for the adipose tissue from the liposuction cannula 900 to the collection canister 102 through the first tube 902, from the collection canister 102 to the inlet 202 of the adipose separation module 200 through the second tube 104, from the inlet 202 to the outlet 204 through the adipose separation module 200, from the outlet 204 of the adipose separation module 200 to the collection reservoir 108 through the third tube 106, and from the collection reservoir 108 to the injector 908 through the fourth tube 110.

In some examples, the first anatomical region and second anatomical region are different anatomical regions in a single subject. In some examples, the subject is a human. In some examples, the adipose tissue is human adipose tissue. In some examples, the first anatomical region comprises an abdomen or a thigh. In some examples, the second anatomical region comprises a breast or a buttock.

For example, the liposuction cannula 900 harvests a mixture comprising fat tissue from a first anatomical region. The first tube 902 fluidly connects the liposuction cannula 900 to the collection canister 102. The collection canister 102 receives and collects the mixture from the liposuction cannula 900 via the first tube 902. The second tube 104 fluidly connects the collection canister 102 to the inlet 202 of the adipose separation module 200. The second tube 104 further fluidly connects the first pump 904 to the collection canister 102 and the adipose separation module 200, such that the second tube 104 communicates a pressure applied by the first pump 904, the pressure being sufficient to: transport the mixture through the second tube 104 from the collection canister 102 to the inlet 202 of the adipose separation module 200. The adipose separation module 200 receives the mixture from the collection canister 102 through the inlet 202 and separates the adipose tissue from the mixture, thereby concentrating the adipose tissue from the mixture. The third tube 106 fluidly connects the outlet 204 of the adipose separation module 200 to the collection reservoir 108. The collection reservoir 108 receives and collects the concentrated adipose tissue from outlet 204 of the adipose separation module 200 via the third tube 106. The fourth tube 110 fluidly connects the collection reservoir 108 to the injector 908. The fourth tube 110 further fluidly connects the second pump 906 to the collection reservoir 108 and the injector 908, such that the fourth tube 110 communicates a pressure applied by the second pump 906, the pressure being sufficient to: transport the concentrated adipose through the fourth tube 110 from the collection reservoir 108 to the injector 908 and inject the concentrated adipose tissue into a second anatomical region.

### Methods

Also disclosed herein are methods of using any of the kits or systems disclosed herein, the methods comprising, for example, transplanting adipose tissue from a first anatomical region to a second anatomical region using any of the kits or systems disclosed herein.

In some examples, the method comprises breast reconstruction, breast augmentation, buttock augmentation, or a combination thereof. In some examples, the method comprises treatment of volume and/or contour abnormality in breast; treatment of congenital breast deformities; or a combination thereof.

### Controllers and User Interfaces

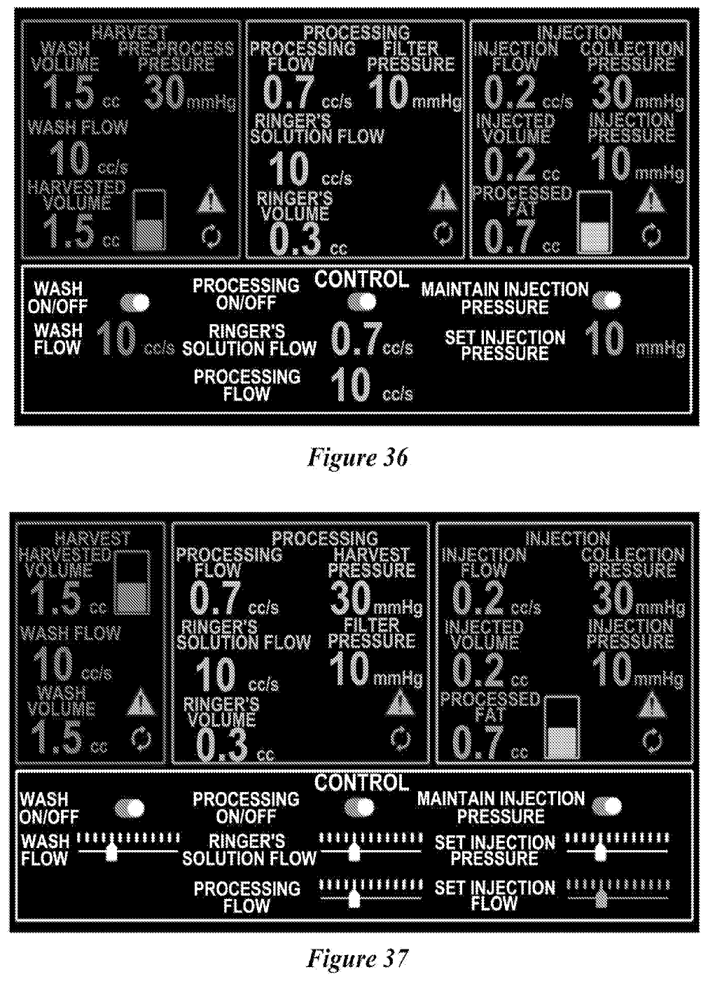

Also disclosed herein are controllers for closed-loop adipose transplant systems. The controller is configured to be communicatively coupled to one or more components of any of the kits or systems disclosed herein. The controller, for example, comprises a user interface comprising a display showing real-time operating parameters and a control selection panel, and the control selection panel displays control parameters and includes: a selector (e.g., a button, an arrow, a slider, etc.) for starting and stopping an adipose transplant procedure upon selection by a user; and one or more selectors for allowing the user to modify one or more of the control parameters. Examples of the user interface are shown in FIG. 36 and FIG. 37.

The real-time operating parameters can, for example, comprise: the volume detected by the first volume sensor (e.g., “the harvested volume”) (when present), the pressure detected by the first pressure sensor (e.g., the “pre-process pressure”) (when present), the pressure detected by the second pressure sensor (e.g., the “filter pressure”) (when present), the pressure detected by the third pressure sensor (e.g., “the collection pressure”) (when present), the pressure detected by the fourth pressure sensor (e.g., the “injection pressure”) (when present), the volume detected by the second volume sensor (e.g., “the processed fat volume”) (when present), the pressure detected by the fifth pressure sensor (e.g., the interstitial pressure) (when present), or a combination thereof.

The control parameters can, for example, comprise: a minimum volume for the first volume sensor, a maximum volume for the first volume sensor, a minimum pressure for the first pressure sensor, a maximum pressure for the first pressure sensor, a minimum pressure for the second pressure sensor, a maximum pressure for the second pressure sensor, a minimum pressure for the third pressure sensor, a maximum pressure for the third pressure sensor, a minimum pressure for the fourth pressure sensor, a maximum pressure for the fourth pressure sensor, a minimum volume for the second volume sensor, a maximum volume for the second volume sensor, or a combination thereof.

In some examples, the controller is further configured to provide feedback to a user when one or more of the real-time operating parameters approaches or exceeds one or more of the control parameters. The feedback can, for example, comprise haptic feedback, auditory feedback, visual feedback, or a combination thereof.

In some examples, the controller is further configured to be communicatively coupled to the means for rotating the rotary implement of the adipose separation module, the first pump, the second pump, or a combination thereof. In some examples, the controller is further configured to adjust the speed of the means for rotating the rotary implement of the adipose separation module, the first pump, the second pump, or a combination thereof such that one or more of the real-time operating parameters stay within the corresponding control parameters. In some examples, the speed of the first pump can be a ratio of the speed of the adipose separation module.

In some examples, the controller is communicatively coupled to the first pump such that when: the volume detected by the first volume sensor (e.g., “the harvested volume”) approaches or exceeds one of the control parameters for the first volume sensor (e.g., the minimum volume or the maximum volume for the first volume sensor), the pressure detected by the first pressure sensor (e.g., the “pre-process pressure”) approaches or exceeds the one of the control parameters for the first pressure sensor (e.g., the minimum pressure or the maximum pressure for the first pressure sensor), the pressure detected by the second pressure sensor (e.g., the “filter pressure”) approaches or exceeds one of the control parameters for the second pressure sensor (e.g., the minimum pressure or the maximum pressure for the second pressure sensor), or a combination thereof; then the controller is configured to adjust the speed of the first pump such that: the volume detected by the first volume sensor stays above the minimum volume or below the maximum volume for the first volume sensor, the pressure detected by the first pressure sensor (e.g., the “pre-process pressure”) stays above the minimum pressure or below the maximum pressure for the first pressure sensor, the pressure detected by the second pressure sensor (e.g., the “filter pressure”) stays above the minimum pressure or below the maximum pressure for the second pressure sensor, or a combination thereof.

In some examples, the controller is communicatively coupled to the means for rotating the rotary implement of the adipose separation module, the first pump, the second pump, or a combination thereof, such that when: the pressure detected by the second pressure sensor (e.g., the “filter pressure”) approaches or exceeds one of the control parameters for the second pressure sensor (e.g., the minimum pressure or the maximum pressure for the second pressure sensor), the pressure detected by the third pressure sensor (e.g., “the collection pressure”) approaches or exceeds on of the control parameters for the third pressure sensor (e.g., the minimum pressure of the maximum pressure for the third pressure sensor), or a combination thereof; then the controller is configured to adjust the speed of the means for rotating the rotary implement, the first pump, the second pump, or a combination thereof, such that: the pressure detected by the second pressure sensor (e.g., the “filter pressure”) stays above the minimum pressure or below the maximum pressure for the second pressure sensor, the pressure detected by the third pressure sensor (e.g., “the collection pressure”) stays above the minimum pressure or below the maximum pressure for the third pressure sensor, or a combination thereof.

In some examples, the controller is communicatively coupled to the second pump, such that when: the pressure detected by the third pressure sensor (e.g., “the collection pressure”) (when present) approaches or exceeds one of the control parameters for the third pressure sensor (e.g., the minimum pressure or the maximum pressure for the third pressure sensor), the pressure detected by the fourth pressure sensor (e.g., the “injection pressure”) (when present) approaches or exceeds one of the control parameters for the fourth pressure sensor (e.g., the minimum pressure or the maximum pressure for the fourth pressure sensor), the volume detected by the second volume sensor (e.g., “the processed fat volume”) (when present) approaches or exceeds one of the control parameters for the second volume sensor (e.g., the minimum volume or the maximum volume for the second volume sensor), or a combination thereof; then the controller is configured to adjust the speed of the second pump such that: the pressure detected by the third pressure sensor stays above the minimum pressure or below the maximum pressure for the third pressure sensor, the pressure detected by the fourth pressure stays above the minimum pressure or below the maximum pressure for the fourth pressure sensor, the volume detected by the second volume sensor stays above the minimum volume or below the maximum volume for the second volume sensor, or a combination thereof.

In some examples, the control selection panel further comprises a selector for starting and stopping flow of the wash liquid into the adipose separation module upon selection by a user. In some examples, the real-time operating parameters further comprise a flow rate for the flow of the wash liquid into the adipose separation module. In some examples, the control parameters further comprise a minimum flow rate for the flow of the wash liquid into the adipose separation module, a maximum flow rate for the flow of the wash liquid into the adipose separation module, or a combination thereof.

In some examples, the real-time operating parameters and/or the control parameters further comprise one or more additional parameters.

In some examples, the controller further comprises a computing device 1000. The detection and/or adjustments of the controller can be carried out in whole or in part on one or more computing device. For example, the controller may comprise one or more additional computing devices.

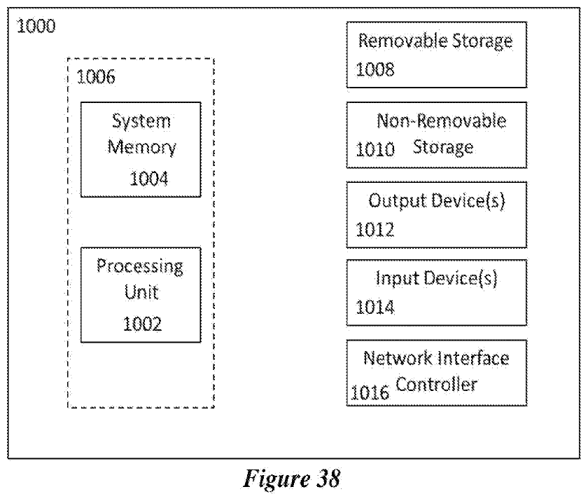

FIG. 38 illustrates an example computing device 1000 upon which examples disclosed herein may be implemented. The computing device 1000 can include a bus or other communication mechanism for communicating information among various components of the computing device 1000. In its most basic configuration, computing device 1000 typically includes at least one processing unit 1002 (a processor) and system memory 1004. Depending on the exact configuration and type of computing device, system memory 1004 may be volatile (such as random access memory (RAM)), non-volatile (such as read-only memory (ROM), flash memory, etc.), or some combination of the two. This most basic configuration is illustrated in FIG. 38 by a dashed line 1006. The processing unit 1002 may be a standard programmable processor that performs arithmetic and logic operations necessary for operation of the computing device 1000.

The computing device 1000 can have additional features/functionality. For example, computing device 1000 may include additional storage such as removable storage 1008 and non-removable storage 1010 including, but not limited to, magnetic or optical disks or tapes. The computing device 1000 can also contain network connection(s) 1016 that allow the device to communicate with other devices. The computing device 1000 can also have input device(s) 1014 such as a keyboard, mouse, touch screen, antenna or other systems configured to communicate with the camera in the system described above, etc. Output device(s) 1012 such as a display, speakers, printer, etc. may also be included. The additional devices can be connected to the bus in order to facilitate communication of data among the components of the computing device 1000.

The processing unit 1002 can be configured to execute program code encoded in tangible, computer-readable media. Computer-readable media refers to any media that is capable of providing data that causes the computing device 1000 (i.e., a machine) to operate in a particular fashion. Various computer-readable media can be utilized to provide instructions to the processing unit 1002 for execution. Common forms of computer-readable media include, for example, magnetic media, optical media, physical media, memory chips or cartridges, a carrier wave, or any other medium from which a computer can read. Example computer-readable media can include, but is not limited to, volatile media, non-volatile media, and transmission media. Volatile and non-volatile media can be implemented in any method or technology for storage of information such as computer readable instructions, data structures, program modules or other data and common forms are discussed in detail below. Transmission media can include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1002 can execute program code stored in the system memory 1004. For example, the bus can carry data to the system memory 1004, from which the processing unit 1002 receives and executes instructions. The data received by the system memory 1004 can optionally be stored on the removable storage 1008 or the non-removable storage 1010 before or after execution by the processing unit 1002.

The computing device 1000 typically includes a variety of computer-readable media. Computer-readable media can be any available media that can be accessed by device 1000 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 1004, removable storage 1008, and non-removable storage 1010 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1000. Any such computer storage media can be part of computing device 1000.

It should be understood that the various techniques described herein can be implemented in connection with hardware or software or, where appropriate, with a combination thereof. Thus, the methods, systems, and associated signal processing of the presently disclosed subject matter, or certain aspects or portions thereof, can take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs can implement or utilize the processes described in connection with the presently disclosed subject matter, e.g., through the use of an application programming interface (API), reusable controls, or the like. Such programs can be implemented in a high level procedural or object-oriented programming language to communicate with a computer system. However, the program(s) can be implemented in assembly or machine language, if desired. In any case, the language can be a compiled or interpreted language and it may be combined with hardware implementations.

In certain examples, the controller comprises a computing device 1000 comprising a processor 1002 and a memory 1004 operably coupled to the processor 1002, the memory 1004 having further computer-executable instructions stored thereon that, when executed by the processor 1002, cause the processor 1002 to carry out one or more of the actions described above for the controller.

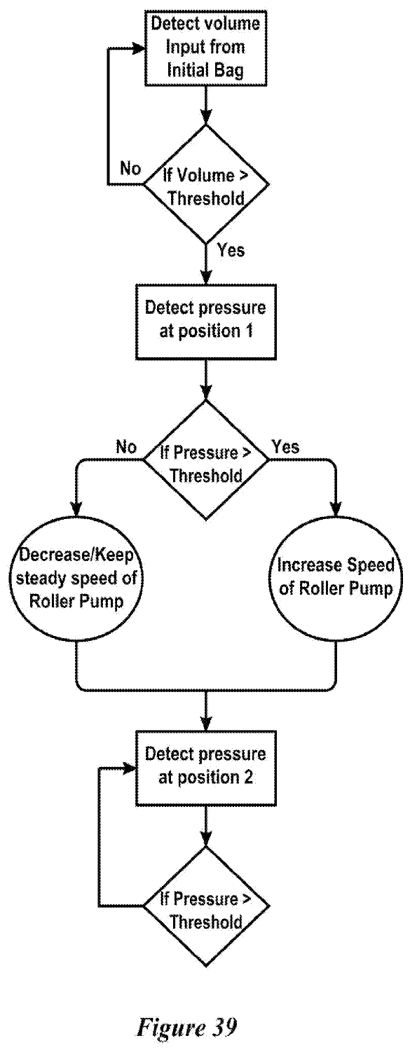

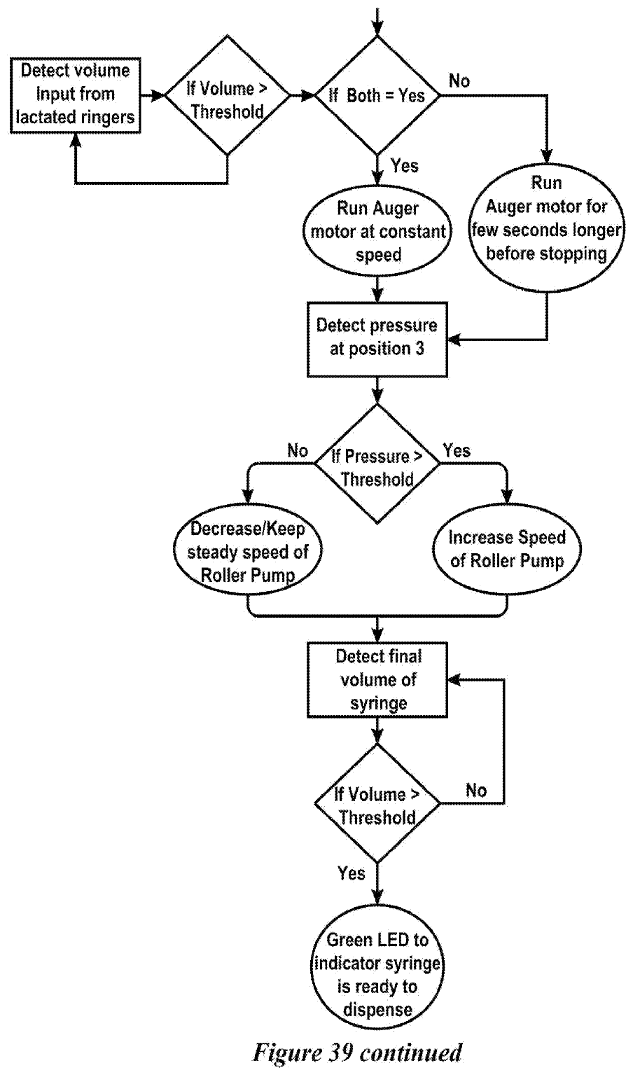

In certain examples, the controller comprises a computing device 1000 comprising a processor 1002 and a memory 1004 operably coupled to the processor 1002, the memory 1004 having further computer-executable instructions stored thereon that, when executed by the processor 1002, cause the processor 1002 to carry our one or more of the actions shown in the flow chart of FIG. 39.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1

Disclosed herein are kits of consumable parts for adipose cell transplant systems, and systems and methods of use thereof.

The Adipose Cell Transplant System is a closed system that comprises three main sections: 1. Fat cell harvest, 2. Fat cell filtration and 3. Injector mechanism.

The fat-cell solution are initially suctioned out of a patient through a liposuction cannula 900, with the suction pressure created by a roller pump 904, which then transports the fat solution into the filtration mechanism 200. An auger 230 can rotate (e.g., in a clockwise direction), transporting the fat solution and applying pressure on a filtration net 210 that surrounds the auger 230, while receiving lactate solution to keep the fat solution moist and flush out the non-fat cell materials. The filtration net 210 then separates the fat cells from the solution and disposes of the excess waste. The filtered fat cells are then be suctioned out by a second roller pump 906 and inserted into an injector 908 that injects the fat cells into the desired anatomical location of the patient.

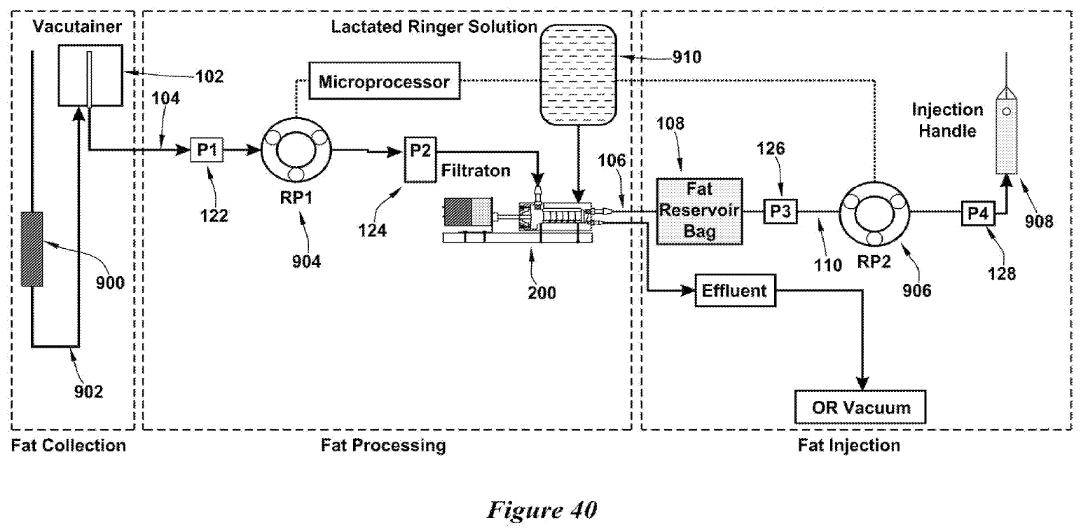

An example system is shown in FIG. 40. This is a closed system comprising three parts. The first part is for fat collection, the second is for fat processing, and the third part is for fat injecting. The first part comprises a suction cannula 900, tubing 902, and collection cannister 102. The second part comprises a first pressure sensor (P1) 122, first roller pump (RP1) 904, fat filtration module 200, and second pressure sensor (P2) 124. The third part contains a fat reservoir bag 108, a third pressure sensor (P3) 126, a second roller pump (RP2) 906, a fourth pressure sensor (P4) 128, and injection handle 908. The fat reservoir bag is between the fat processing and fat injection components.

Two roller pumps are used to move the fat cells through the system. Typically, other systems use pressure filtration (syringe pressure for example) or centrifugation, which both use of pressure, which may be damaging is not ideal for transferring fat cells.

The two pump design offers the ability to harvest and process the fat at the same time in a closed system, which is not available with any products currently on the market. The first roller pump (RP1) 904 is used to harvest the fat mixture and transport the fat mixture from the vacutainer 102 into the fat filtration module 200. The second pump (RP2) 906 is used to propel the harvested fat from the reservoir bag 108 to the injector 908 upon surgeon actuation, e.g., upon actuation of a switch on the injector handle 908.

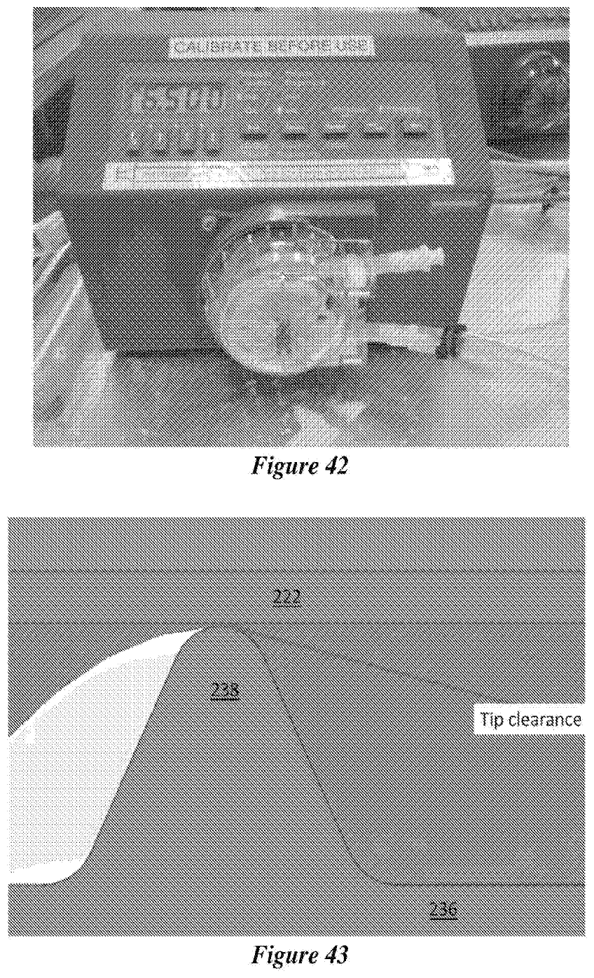

An example image of a roller pump using medical grade tubing is shown in FIG. 42.

A reservoir storage bag 108 that is located between the two roller pumps permits the pumps to have the capacity to operate at different speeds. This feature can be used to optimize the control of harvest speed to what is required by the surgeon, eliminating waiting time for fat filtering and processing. Processing and injecting do not need to occur at the same rate because of the reservoir bag 108 which is between the fat filtration module 200 and the injector 908.

The fat filtration module 200 is the main filtration mechanism for the system. In some examples, the fat filtration module 200 includes a rotary implement 230, such as an auger, that pumps the fat cells along the surrounding filter 210 to concentrate the fat and remove the liquid portion. The auger 230 can also serve as a cleaning function for the filter 210, creating a wiping function as it rotates. The auger 230 can, in some examples, have a variable pitch. In some examples, the auger 230 cab be attached to a titanium rod 244, which can then be attached to a flexible shaft coupler that securely holds the motor shaft and auger 230. The auger 230 can be made of polycarbonate, and has a very smooth surface finish, with a rigid, strong body. The fat cell solution will rotate within the auger 230, allowing the solution to be moist from the Lactated Ringers while sifting out all the excess waste. The auger-motor assembly can be placed within a titanium rod.

The motor 228 can be a gear motor with a controllable speed such as from 2 to 25 RPM, or from 15-20 RPM.

The wash liquid, such as lactated ringers solution, can enter the fat filtration module 200 through an orifice 205, optionally together with 0.25 inch tubing and a 1/16 NPT fitting, and can be controlled by a pressure transducer.

The first roller pump 904 injects the mixture into the fat filtration module 200 through an inlet 202 optionally together with tubing. The amount of pressure and volume of the mixture in the fat filtration module 200 can be monitored. The speed of rotation of the auger 230 can be adjusted based on the amount of the mixture injected into the fat filtration module 200. The flow rate of the first roller pump 904 can be adjusted, for example between 20 mL/min to 200 mL/min, such as from 50 to 75 mL/min.

The lactated ringers solution can contact the mixture in the fat filtration module. The rate the lactated ringers solution flows into the fat filtration module can be controlled based on how much “washing” of the mixture is desired.

Excess waste materials and lactated ringers solution pass through the filter 210 into a second compartment 214 in the fat filtration module 210, which can then transport the waste towards a port 203.

The filtered adipose cells can exit the fat filtration module 200 through an outlet 204. In some examples, the outlet 204 is positioned at a right angle relative to the longitudinal axis of the auger 230. The amount of pressure applied to the filtered adipose cells by the second roller pump 906 can be controlled by variable parameters in a control panel. The flow rate of the second roller pump 906 can be adjusted, for example between 10 and 180 mL/min, such as from 60 to 90 mL/min.

The waste material (e.g., effluent) can proceed through the port 203 and optionally through 3/16 tubing and 1/16 NPT fitting.

The spacing between the edge of the auger 230 and the interior cavity 208 in the fat filtration module 200 can be adjusted to improve the pumping performance of the auger 230 and minimize dead space.

The near-tight fitting of the auger 230 inside the interior cavity 208 can allow the fat cells to move with enough pressure to force out the excess waste from the fat solution, as well as not too much pressure to where the fat cells will break. The auger 230 can be positioned along the centerline of the filter cage 222 and nylon mesh filter 210 with a small clearance between the auger 230 and the filter 210 (“tip clearance”), which allows for efficient separation of the adipose cells from the mixture, without damage to the adipose cells. The auger rod 244 and shaft coupler can be positioned outside the housing 206, allowing access to the gear motor 228 for easy changes. Therefore, the fat filtration module 200 can be readily removed from the motor 228 so that it can be disposed properly and filter 210 can be changed.

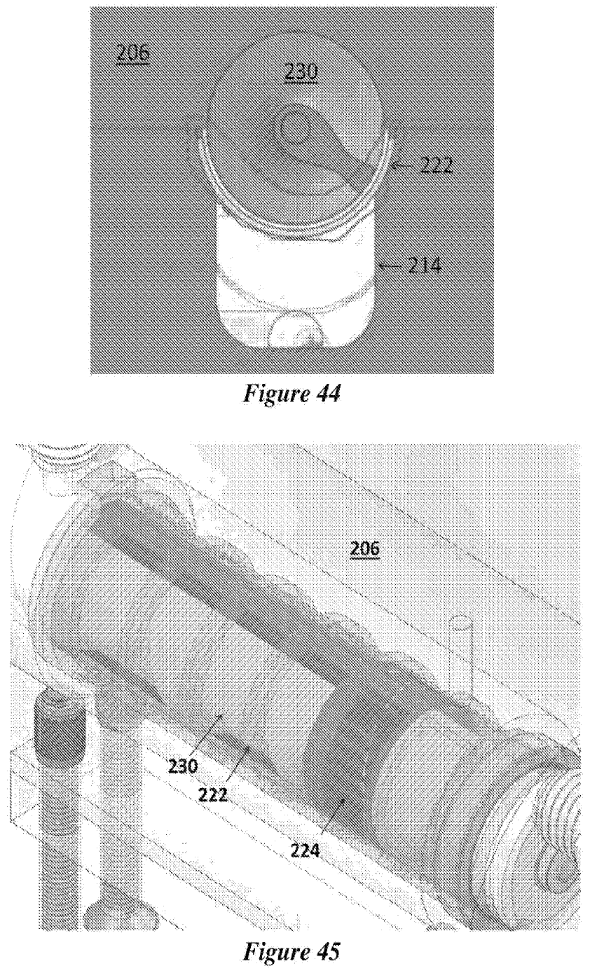

As shown in FIG. 43 and FIG. 44, there is a small space between the edge of the blade 238 of the auger 230 and the filter cage 222. Within this space, the nylon mesh filter 210 can be held. When fixed in place, the nylon mesh filter 210 can make a cylinder disposed circumferentially about and coaxially with the auger 230, and can contain the fat cell solution.

In some examples, the auger 230 can comprise a variable pitch auger 230. The variable pitch of the auger 230 can optimize the pumping of semi solids as liquid is extracted. A formula can be used to estimate volumetric flow through the auger by using a linear rate of change in the auger pitch, using variables for total flow through the auger:

$$Q = \frac{π}{4} [ {OD}^{2} - {ID}^{2} ] ω \overline{P}$$

$$\overline{P} = \frac{1}{L} [ \frac{α {L}^{2}}{2} + {P}_{1} L ] = \frac{α L}{2} + {P}_{1}$$

$$α = \frac{{P}_{1} - {P}_{2}}{L}$$

Where Q is the volumetric flow rate (mL/s), P₁ is the pitch of the auger at the inlet (inches/revolution), P₂ is the pitch of the auger at the outlet (inches/revolution), L is the length of the auger (inches), ω is the motor speed (revolution per minute), OD is the outer diameter of the auger (inches), and ID is the inner diameter of the auger (inches).

This equation assumes a linear pitch rate change over the auger length. The two biggest contributors to the volumetric flow is the annular area controlled by the outer diameter (OD) and inner diameter (ID) of the auger, and the rotational speed of the auger. The average pitch can be used to calculate the speed of the media through the auger.

An example of a variable pitch auger 230 is shown in FIG. 6.

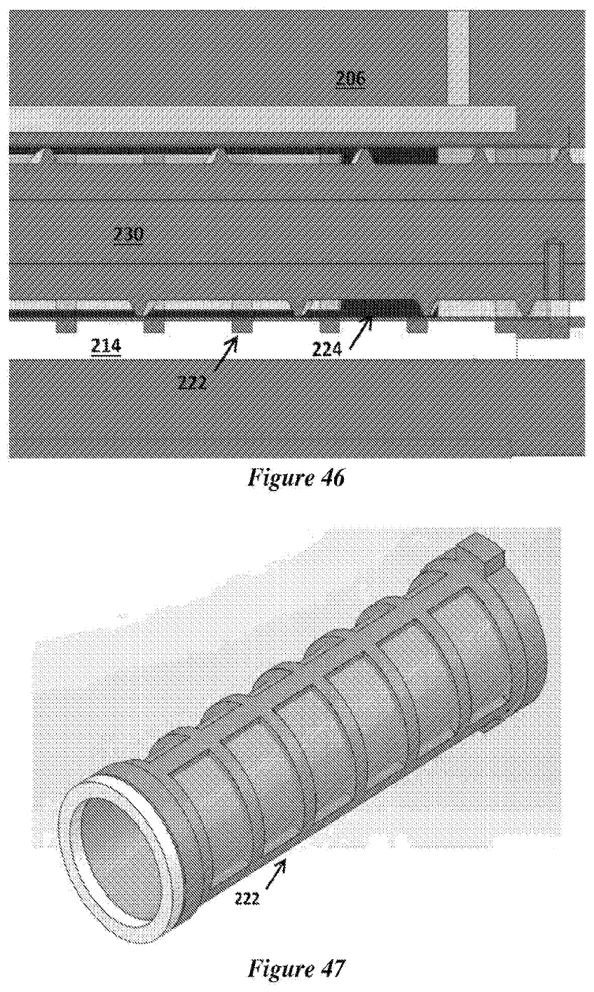

An optional sliding ring 224 can be added to the fat filtration module 200 to facilitate the clearing action of the auger 230. An example fat filtration module 200 including the sliding ring 224 is shown in FIG. 13, FIG. 45, and FIG. 46.

A cage 222 surrounds the auger 230 that holds the filter 210, which can be metal mesh or any filter material. This permits the use of a variety of different filter sizes. Variable filter sizes can be easily changed to the preference of a user. For example, different micron sized filters can be selected for types of fat in different body locations (legs, abdomen, back, etc.), as these different body areas can have different textures of fat. The filter material (polymer or metal, such as titanium) can be an flexible or pliable material that is used in conjunction with the cage 222 for support.

The cage 222 around the filter 210 can be customized. For example, the cage 222 can be a flexible polymer cage, a rigid cage, etc. An example of a filter cage 222 is shown in FIG. 47.

The use of the filter cage makes the filter a cartridge style set up. For example, the use of the filter cage provide the ability to change the filter during a treatment intraoperatively in the sterile field. This feature can allow operative staff to quickly change filters during a treatment in case different micron sizes were needed or in the event that the filter became clogged. This enables the filter to be changed out as many times as needed in any given surgical case.

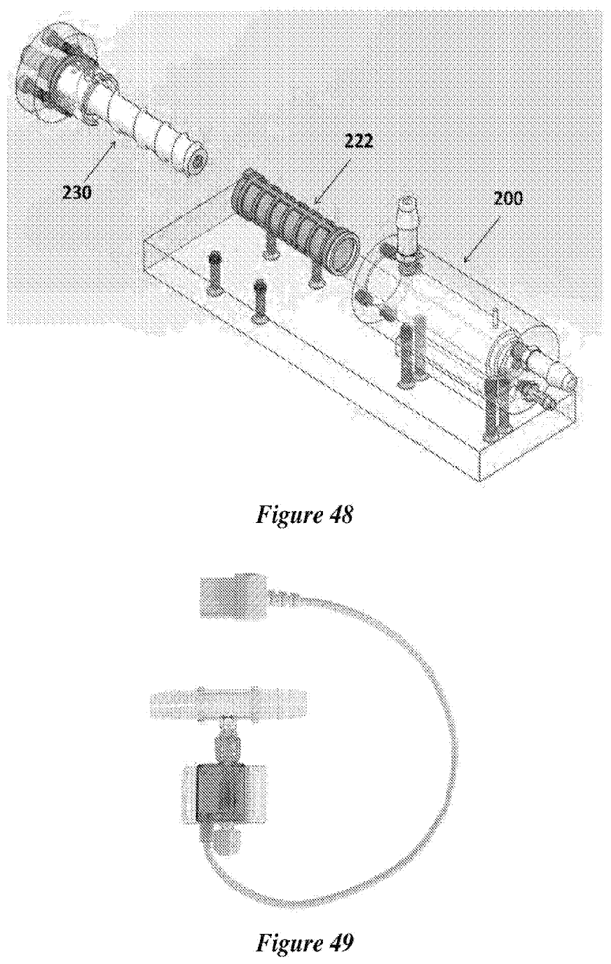

An example exploded view of the fat filtration module 200 including the auger 230 and filter cage 222 is shown in FIG. 48.

The systems also include the ability to add a secondary filter (e.g., an effluent separation module 300) to process the effluent with a smaller pore filter and capture additional PRP and/or stem cells and add them back to the fat cells for injection to optimize their take.

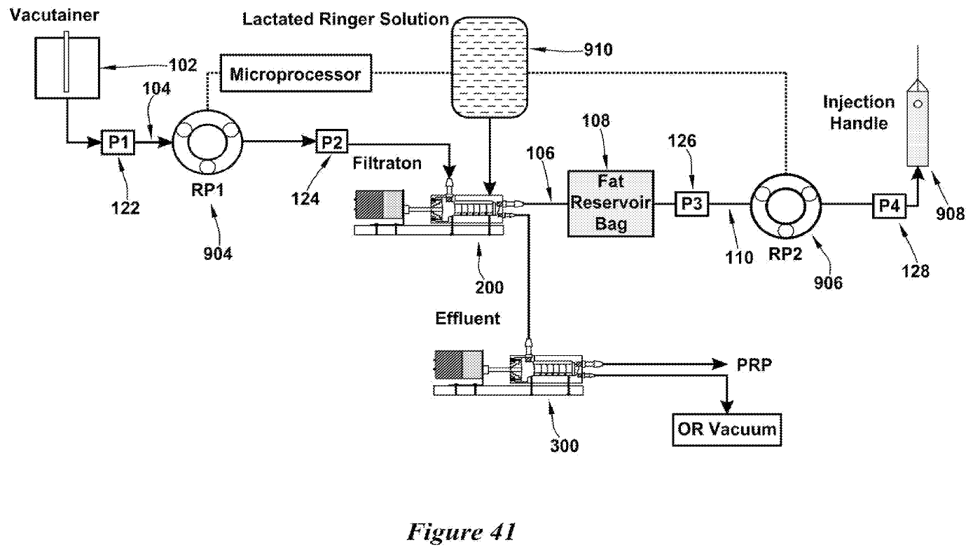

The fat filtration module 200 includes two outflow components, a port 203 which goes to a collection bag for PRP and stem cells, and an outlet 204 which takes the fat to the injector handle 208. An example system including an effluent separation module 300 is shown in FIG. 41. The whole system can be two stages, the first for fat concentration and the second for filtering of the effluent. The second stage filter can be an effluent separation module including an auger, or a membrane filter.

The fat concentration can be controlled by the differential speed between the first roller pump 904 and the auger 230, thereby controlling the viscosity of the transplanted fat.

The system can include pressure sensors, such as a first pressure sensor 122, a second pressure sensor 124, a third pressure sensor 126, and a fourth pressure sensor 128 as shown in FIG. 40 and FIG. 41.

The first pressure sensor 122 can be located between the collection canister 102 and the first roller pump 904. The first pressure sensor 122 can detect whether or not there is liposuction material in the collection canister 102. Pressure bounds can be set so that the fat cells are not damaged by excessive negative pressure. The system can modulate the speed of the first roller pump 904 to stay within the pressure bounds.

The second pressure sensor 124 can be located between the first roller pump 904 and the fat filtration module 200. The second pressure sensor 124 can help the system determine the change in pressure over time, and also help regulate the speed ratio of the first roller pump 904 to the auger 230 so there is always sufficient material to keep the filtration at the correct operating speed. The second pressure sensor 124 can act as a safety mechanism; if there is no place for liquid to go (e.g., fat filtration module 200 is clogged or collection reservoir 108 is full), the system will slow down or stop the first roller pump 904.

The third pressure sensor 126 can be located between the collection reservoir 108 and the second roller pump 906. The third pressure sensor 126 can detect if there is concentrated fat available to be dispensed by the surgeon. The third pressure sensor 126 can also detect if the collection reservoir 108 is full.

The fourth pressure sensor 128 can be located between the second roller pump 906 and the injector 908. The fourth pressure sensor 128 can detect and control the injection pressure of the fat. Closed loop control on this pressure can be implemented to maintain constant injection pressure to provide precise dispensing control.

The system can further include a pressure sensor that detects the tissue interstitial pressure at the injection site. Pressure sensors/control at the injection site can prevent injecting into the muscle or vasculature. This is a safety feature that can prevent dangerous fat emboli. The pressure sensor can be built into the injector handle, which can further display the detected pressure.

The pressure sensors can, for example, be teed off of the tubing so that it does not clog. An image of an example pressure sensor is shown in FIG. 49.

A pressure system logic diagram is shown in FIG. 39.

An instrumentation control panel serves as a digital dashboard for the fat grafting system. The thickness of the fat (wet, dry, etc.) is controlled by the speed of the fat filtration module. The rate of filtration also influences the concentration of the fat. Two filters can be set up in parallel, and the first and second filtrate can be controlled. Pressures can be visualized with data from the pressure sensors. Different gauges can show pressure and volume readings at different locations. The panel can show the entire system is working and what is occurring at various locations throughout the system.

Example user interfaces for the controllers are shown in FIG. 36 and FIG. 37.

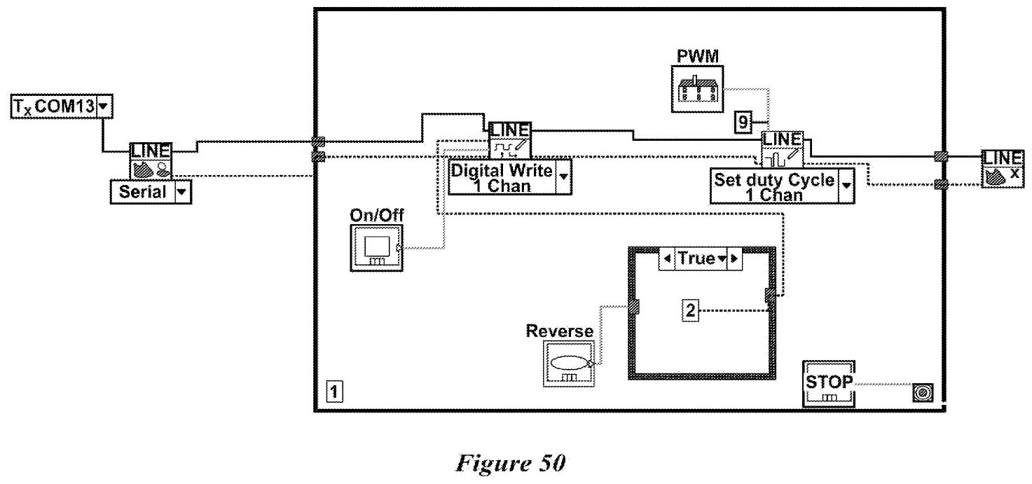

An example block diagram for motor/pump control is shown in FIG. 50. The software can control the speed of the motor/pump. Various digital and analog signals can be taken from pressure transducers and volume detectors. All variables can be inserted into a block diagram, like the one shown in FIG. 50, to automate how much fat cell solution and filtered solution is going through the fat-cell separator 200.

Rates of processing can be controlled, which controls the fat concentration: rate of processing (speed of first roller pump and auger), control of concentration (ratio of first roller pump and auger speed), rate of injection (second roller pump), closed loop control of interstitial injection pressure (second roller pump and fourth pressure sensor), safety thresholds of pressure sensors (tissue pressure, fourth pressure sensor), and safety thresholds for the first, second, and third pressure sensors (not too high or too low, to avoid damaging fat cells).

In some examples, the injector 908 comprises an ergonomic injection handle, such as the one shown in FIG. 35. The injection handle can be ergonomically designed to optimize the tactile control and precision for fat cell injection. The ergonomic injection handle can include a single button control for automatic injection with feedback for pressure threshold control. The feedback could be a vibration motor inside of the handle (haptic feedback), an audible annunciator (auditory feedback), or LED (visual feedback). The second roller pump generates pressure so that injection with a traditional syringe is not required by the surgeon, thereby lessening the physical burden that is currently required. An option for retractable injector needle with pressure fail safes is possible. The surgeon will also have the ability to lock the retractable needle in place. Safety features using pressure sensors can be important for preventing fat embolism in patients, and does not currently exist in any system on the market.

The ergonomic injection handle can be designed to be used with standard available cannulas. The ergonomic design can be optimized for surgical performance. A single button can control the second roller pump.

### Example 2

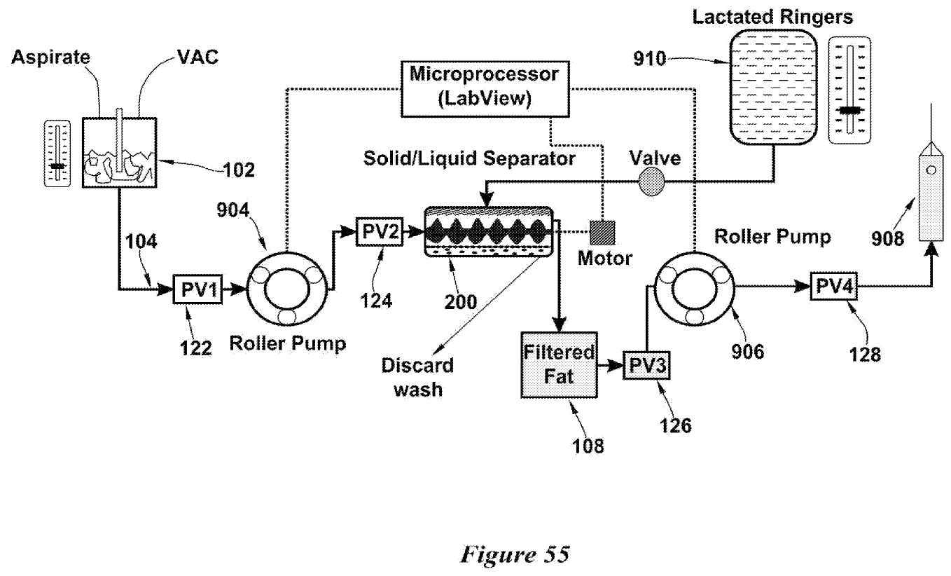

Disclosed herein are fat-cell transplant systems, for example as shown in FIG. 55. The fat-cell solution can be suctioned out of a patient through a cannula 900, with the suction pressure created by a roller pump that is controlled, for example, through LabVIEW. A roller pump 904 can then transport the fat solution into the fat filtration module 200. An auger can rotate, for example in a clockwise direction, transporting the fat solution and applying pressure on a filtration net that surrounds the auger, while receiving lactate solution to keep the fat solution moist and flush out the non-fat cell materials. The filtration net 210 can then separate the fat cells from the solution and dispose of the excess waste. The filtered fat cells can then be suctioned out by a second roller pump 906 and inserted into an injector 908 that can inject the fat cells into the desired anatomical location of the patient.

The roller pumps and motor rotating the auger can be controlled through LabVIEW. Sterile pressure transducers can be in line at locations PV1, PV2, PV3, etc. Based on the pressure detected at each location, the speed of the pumps and motors can be automatically controlled through LabVIEW software. At location PV4, the value of the pressure detected can trigger a switch that allows the injector to begin exporting the filtered fat cells.

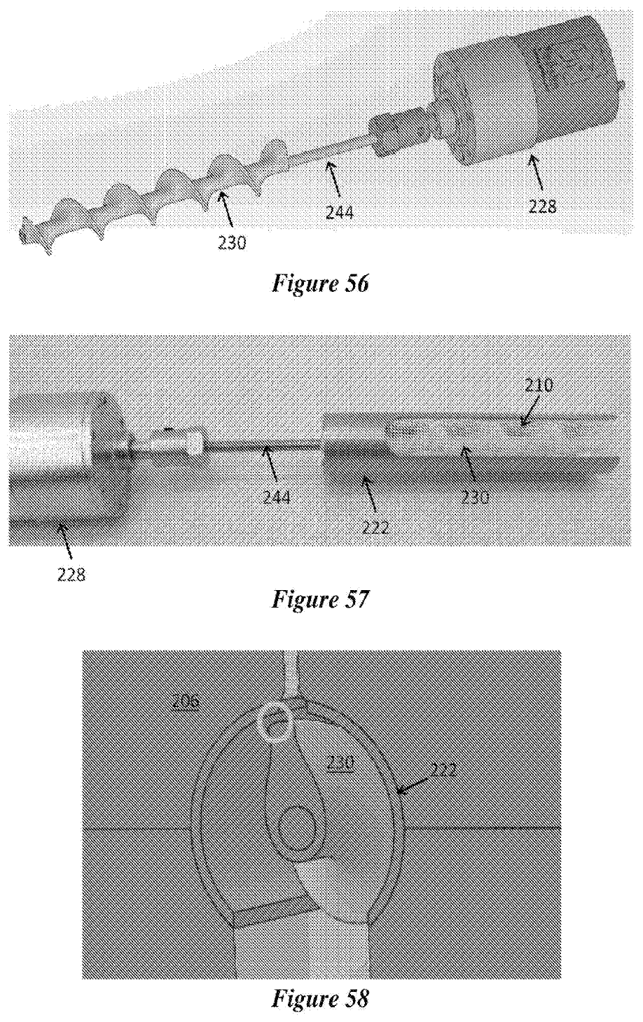

The auger 230 can, for example, be 3-D printed from Accura Xtreme White Normal-Resolution Stereolithography (SLA) material and can be attached to a M3-threaded titanium rod 244, which can then be attached to a motor hex coupler that securely holds the motor shaft and auger, as depicted in FIG. 56. The motor 228 can, for example, be a 30 RPM gear motor, which can also be controlled by LabVIEW.

The auger-motor assembly can be placed within a ¾″ titanium cage 222, as depicted in FIG. 57, where the mesh-filter 210 can be attached to the interior of the cage 222 to filter out the fat cells. The length of the cage 222 can be as long as the auger 230 itself, for example 10 cm. The near-tight fitting of the auger 230 inside the cage 222 can allow the fat cells to move with enough pressure to force out the excess waste from the fat solution, as well as not too much pressure to where the fat cells will break. This small clearance is depicted in FIG. 58, where the green circle depicts the location of the clearance where the filter 210 will be placed, The clearance can, for example, be 1 millimeter (mm) or less.

The titanium cage 222 can sit inside a polycarbonate housing 206 for the filtration mechanism 200. There are various inlet and outlet ports on the system 200.

The adipose separation module 200 can include a second compartment 214 where the excess solution (e.g., effluent) that was filtered out by the auger and mesh filter is collected. The module 200 can include a port 203 fluidly connected to the second compartment 214 for removal of the effluent.

The adipose separation module 200 can include an orifice 205 for introducing a wash liquid, such as lactated ringers, into the mixture as it goes through the auger. While the auger rotates the solution around to apply pressure to filter out excess waste, the lactate solution will slowly drip throughout the fat mixture to keep the solution moist, easing the flow through the auger and making the filtration process easier.

The Lactate solution and fat-cell solution can be inserted into the filtration system 200 through ¼″ standard tubing, while the effluent and filtered fat cells can exit through 3/16″ tubing. The primary reason for different sizes is to help identify where each piece of the system goes and to take caution by not allowing too easy of a flow or too tight of low, which could damage the fat cells. The 3/16″ tubing can go into a leur lock that connects to an injector and cannula needle that will reinsert the fat cells into the host patient.

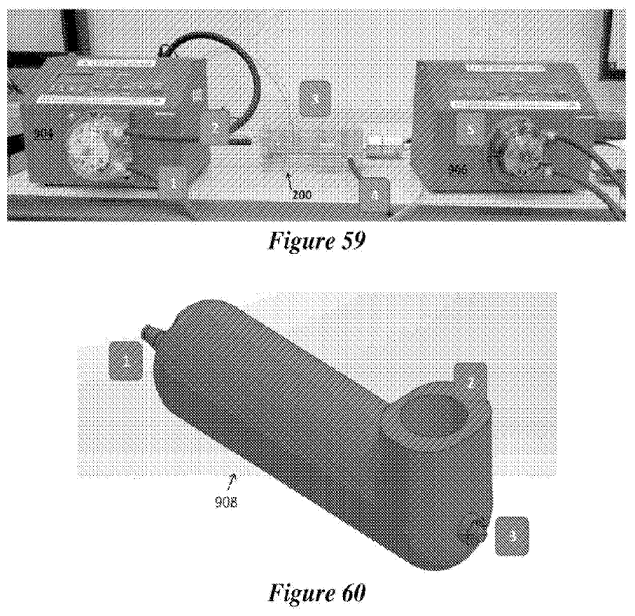

FIG. 59 depicts the entire system with detailed labels for each location shown. Location descriptions: (1) fat cell solution can be pumped 904 into here, (2) a pressure transducer can detect the fat solution, initiating the release of lactate solution and rotation of auger, (3) Filtration System 200, (4) excess waste can leave this direction, and (5) filtered fat cells can be suctioned through this pump 906 into the injector.

FIG. 60 is a schematic diagram for the injector 908 that can be used be used to reinsert the fat cells, which can be 3-D printed. Location descriptions: (1) 3/16 ID Tubing Barbed fitting, inlet hole for filtered Fat Solution, (2) location of an on/off switch, user control fat cell pump, and (3) leur locking mechanism for cannula of choice. The needle is approximately 1.2 inches in diameter, with finger grooves at the bottom to allow ease of hold. The injector can also include a switch, which can allow the user to control when to pump in the filtered fat cells.

### Example 3

Described herein are systems and kits for fat grafting, such as autologous fat grafting. Autologous fat grafting is a surgical procedure that involves harvesting, processing, and transferring adipose tissue from one anatomical region of the patient to another.

Fat grafting is mainly used for the treatment of volume and contour abnormalities and congenital breast deformities. It is a technically demanding and time-consuming procedure involving several steps to harvest, process, and transfer fat. Current fat grafting techniques lack integration of the components in each step.

A whole closed system could potentially optimize operating times and automation of the injection of the cells that control both the injection rate and corresponding needle retraction to improve the ease and efficacy of fat grafting.

Disclosed herein is a closed system for fat cell transplant including: collection of fat tissue through liposuction, washing the mixture to remove non-fat cell biologic waste, concentrating only the fat cells, and then re-injecting the concentrated fat cells back into the patient where they are needed. The specific device that washes and concentrates the fat cells can include an auger mechanism within a filter screen where the fat cells are moved along the auger while the other cells and washing fluid fall through the filter screen.

Commercial systems require manually squeezing the waste material through a screen embedded in a IV-like bag. This is a subjective process, requires additional personal and replacement of bags and tubing exposing the concentrated fat cells to the open air. The closed system disclosed herein can be motor driven, reduce the clinical supported required, allow continuous liposuction and fat cell transplant, and minimize the chances of contamination of the concentrated fat cells.

### Example 4

Fat grafting has increased in popularity as is a major adjunct in breast reconstruction and breast augmentation. Currently there are few devices to facilitate fat graft transfer, all have significant limitations. These limitations include intraoperative inefficiency, inability to graft and process at the same time, ergonomics, manual injection, and quality of fat harvested. To solve these problems, an all in one closed system is described herein that allows processing and harvesting fat with an automatic injection handle that has a pressure sensor to fine tune the fat injection process. This can allow concurrent harvesting and processing of fat with precise control of pressure which can be important for fat cell integrity.

Autologous fat grafting is a surgical procedure that involves harvesting, processing, and transferring adipose tissue from an anatomical region of the patient to another. Over the past decade, autologous fat grafting has become a widespread procedure, according to the American Society of Plastic Surgeons (ASPS), 31,862 fat grafting procedures were performed for breast reconstruction during 2017 in the U.S. Furthermore, it is considered the plastic surgery technique which has evolved the most over the past 30 years (American Society of Plastic Surgeons (ASPS). 2017 Plastic Surgery Statistics Report. Available from: https://www.plasticsurgery.org/news/plastic-surgery-statistics. Published 2017).

Specifically, in breast reconstruction, fat grafting is mainly used for the treatment of volume and contour abnormalities, as well as for more severe cases of congenital breast deformities. It also improves skin texture and scarring especially in post-radiated reconstruction.

Although, it is a safe procedure with low complication rates, it is technically demanding and time-consuming since it involves several steps to transfer the adipose tissue to the desire anatomical location successfully. The steps involved are as follows: (1) Harvesting: with the help of a liposuction cannula, the desired amount of fat is suctioned from the patient. (2) Processing: This involves different techniques to filter the viable adipose tissue from plasma, blood remnants, and lysed adipocyte cells. (3) Fat transfer: This is the last step and involves the use of a syringe that controls the flow rate and volume of fat that is transferred.

Current fat grafting technologies lack integration in the components of each step, meaning that technology has been developed for each element rather than for a whole closed system that could potentially optimize operating times and the plastic surgeon fat grafting experience. Disclosed herein is a closed fat grafting system that can optimize operating times during fat grafting procedures.

The bioavailability of the processed fat grafting using the systems and methods herein can be assessed using a well-described mouse model performing xenografts using human fat (Lujan-Hernandez J et al. Experimental in-vivo models used in fat grafting research for volume augmentation in soft tissue reconstruction. Arch Plast Surg. 2017; 44(5):361-369).

### Example 5

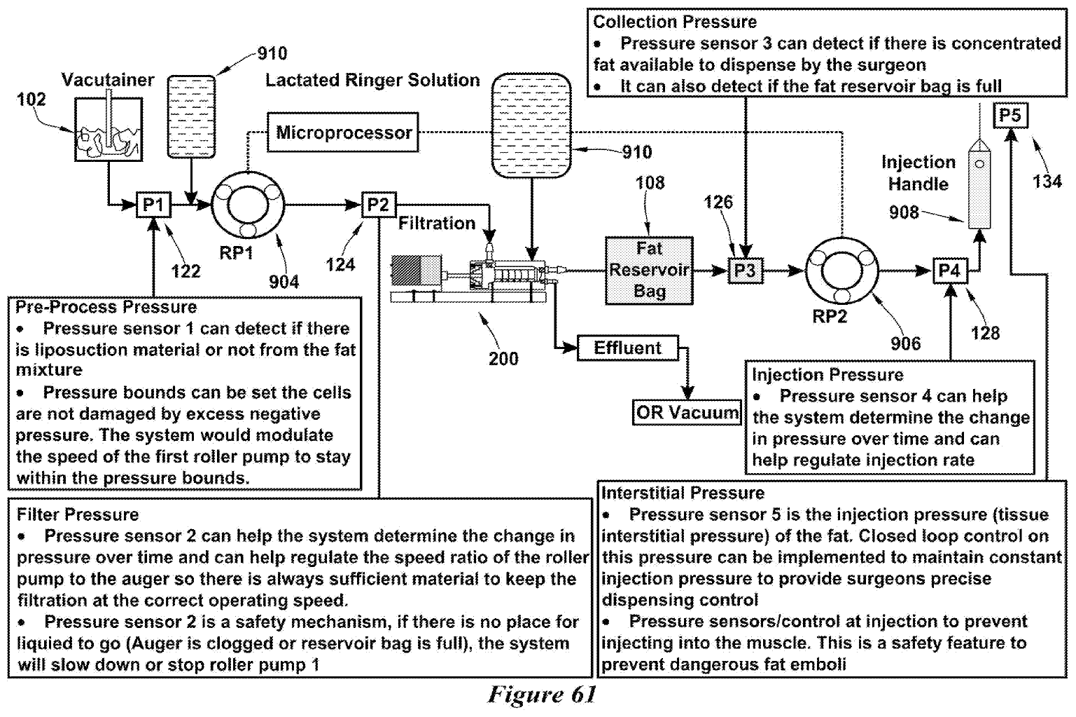

An example system is shown in FIG. 61. The fat-cell solution can be suctioned out of a patient through a cannula and transferred into a collection cannister 102.

A roller pump 904 can then transport the fat solution into the fat filtration module 200. In some examples, the fat solution can be washed with a wash liquid between the collection cannister and the fat filtration module, for example prior to the first roller pump.

In the fat filtration module 200, an rotary implement can rotate, for example in a clockwise direction, transporting the fat solution and applying pressure on a filter that surrounds the rotary implement, while receiving lactate solution to keep the fat solution moist and flush out the non-fat cell materials. The filter 210 can then separate the fat cells from the solution and dispose of the excess waste. The filtered fat cells can then be suctioned out by a second roller pump 906 and inserted into an injector 908 that can inject the fat cells into the desired anatomical location of the patient.

The systems also include the ability to add a secondary filter (e.g., an effluent separation module 300) to process the effluent with a smaller pore filter and capture additional PRP and/or stem cells and add them back to the fat cells for injection to optimize their take.

The system can further include a pressure sensor (P5) 134 that detects the tissue interstitial pressure at the injection site. Pressure sensors/control at the injection site can prevent injecting into the muscle or vasculature. This is a safety feature that can prevent dangerous fat emboli. The pressure sensor can be built into the injector handle, which can further display the detected pressure.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A kit of consumable parts for a closed-loop adipose transplant system including a liposuction cannula, a first tube, a first pump, a second pump, and an injector, the kit comprising:
a collection canister;
a second tube;
an adipose separation module having an inlet and an outlet;
a collection reservoir;
a third tube; and
a fourth tube;
wherein the second tube is configured to fluidly connect the collection canister to the inlet of the adipose separation module;
wherein the third tube is configured to fluidly connect the outlet of the adipose separation module to the collection reservoir;
wherein the fourth tube is configured to fluidly connect the collection reservoir to the injector;
wherein the collection canister is configured to receive and collect a mixture comprising adipose tissue harvested from a first anatomical region by the liposuction cannula via the first tube;
wherein the adipose separation module is configured to: receive the mixture from the collection canister through the inlet and separate the adipose tissue from the mixture, thereby concentrating the adipose tissue from the mixture;
wherein the collection reservoir is configured to receive and collect the concentrated adipose tissue from outlet of the adipose separation module via the third tube; and
wherein the second tube is configured to fluidly connect the first pump to the collection canister and the adipose separation module, such that the second tube communicates a pressure applied by the first pump, the pressure being sufficient to: transport the mixture through the second tube from the collection canister to the inlet of the adipose separation module;
wherein the fourth tube is configured to fluidly connect the second pump to the collection reservoir and the injector, such that the fourth tube communicates a pressure applied by the second pump, the pressure being sufficient to: transport the concentrated adipose through the fourth tube from the collection reservoir to the injector and inject the concentrated adipose tissue into a second anatomical region;
wherein the consumable parts together form a continuous, closed fluid pathway for the adipose tissue from the liposuction cannula to the collection canister through the first tube, from the collection canister to the inlet of the adipose separation module through the second tube, from the inlet to the outlet through the adipose separation module, from the outlet of the adipose separation module to the collection reservoir through the third tube, and from the collection reservoir to the injector through the fourth tube.

2. (canceled)

3. (canceled)

4. (canceled)

5. (canceled)

6. The kit of claim 1, wherein the adipose separation module is further configured to contact the mixture with a wash liquid, thereby washing the mixture, and to separate the adipose tissue from the mixture and the wash liquid, thereby concentrating the adipose tissue.

7-11. (canceled)

12. The kit of claim 1, wherein the adipose separation module further comprises:
a housing defining an interior cavity, the inlet, and the outlet;
a filter configured to be disposed within the interior cavity, such that the filter is configured to define a first compartment and a second compartment within the interior cavity, the first compartment being a portion of the interior cavity encompassed by the filter and the second compartment being a portion of the interior cavity outside the filter;
the first compartment having a proximal end and a distal end;
the inlet of the adipose separation module being fluidly connected to the first compartment at or near the proximal end of the first compartment;
the outlet of the adipose separation module being fluidly connected to the first compartment at or near the distal end of the first compartment;
the orifice of the fat separation module, when present, being fluidly connected to the first compartment;
the filter being configured to separate the adipose tissue from the wash liquid (when present) and other components in the mixture by passing the wash liquid (when present) and the other components from the mixture through the filter into the second compartment as the mixture is transported through the housing, thereby concentrating the adipose tissue within the first compartment and forming an effluent in the second compartment, the effluent comprising the wash liquid (when present) and the other components from the mixture.

13-24. (canceled)

25. The kit of claim 12, wherein the first compartment is disposed coaxially with the filter.

26-38. (canceled)

39. The kit of claim 12, wherein the adipose separation module further comprises a filter cage disposed circumferentially around and coaxially with the filter within the interior cavity.

40. (canceled)

41. (canceled)

42. (canceled)

43. The kit of claim 12, wherein the adipose separation module further comprises:
a rotary implement having a proximal end and a distal end, the rotary implement comprising a central shaft and a blade extending from the central shaft;
wherein the filter is configured to be disposed circumferentially around and coaxially with the rotary implement within the interior cavity, such that the rotary implement is configured to be rotatably disposed within the first compartment with the proximal end of the rotary implement disposed towards the proximal end of the first compartment and the distal end of the rotary implement being disposed towards the distal end of the first compartment; and
wherein the rotary implement is configured to agitate the mixture within the first compartment via rotation of the rotary implement.

44. (canceled)

45. (canceled)

46. (canceled)

47. (canceled)

48. The kit of claim 43, wherein the blade of the rotary implement has an edge, and wherein the edge and the filter are radially spaced apart from each other by a distance of from 0 μm to 2 mm.

49-61. (canceled)

62. The kit of claim 43, wherein the adipose separation module further comprises a sliding ring, the sliding ring being slidably disposed between the rotary implement and an inner surface of the filter, such that the sliding ring is disposed circumferentially around and coaxially with the rotary implement and the inner surface of the filter is disposed circumferentially around and coaxially with the sliding ring, wherein the sliding ring is configured to slide axially to clear the inner surface of the filter.

63-70. (canceled)

71. The kit of claim 12, wherein the adipose separation module further comprises a port defined by the housing, the port being fluidly connected to the second compartment.

72. (canceled)

73. (canceled)

74. The kit of claim 71, wherein the kit further comprises a sixth tube and an effluent receptacle, wherein the sixth tube is configured to fluidly connect the port of the adipose separation module to the effluent receptacle, the effluent receptacle being configured to receive the effluent from the port of the adipose separation module.

75. The kit of claim 71, wherein the kit further comprises a seventh tube and an effluent separation module having an inlet and an outlet, wherein the seventh tube is configured to fluidly connect the inlet of the effluent separation module to the port of the adipose separation module, the effluent separation module being configured to receive the effluent from the port of the adipose separation module and separate a desired component from the effluent.

76. (canceled)

77. (canceled)

78. (canceled)

79. (canceled)

80. The kit of claim 75, wherein the effluent separation module comprises a membrane filter, the membrane filter being configured to separate the desired component from other components in the effluent by passing the desired component through the membrane filter and blocking the passage of the other components through the membrane filter.

81. The kit of claim 75, wherein the effluent separation module comprises:
a housing defining an interior cavity, the inlet, and the outlet;
a filter configured to be disposed within the interior cavity, such that the filter is configured to define a first compartment and a second compartment within the interior cavity, the first compartment being a portion of the interior cavity encompassed by the filter and the second compartment being a portion of the interior cavity outside the filter;
the first compartment having a proximal end and a distal end;
the inlet of the effluent separation module being fluidly connected to the first compartment at or near the proximal end of the first compartment;
the outlet of the effluent separation module being fluidly connected to the first compartment at or near the distal end of the first compartment;
the filter being configured to separate the desired component from the other components in the effluent by passing the other components from the effluent through the filter into the second compartment as the effluent is transported through the housing, thereby concentrating the desired component within the first compartment and forming a waste solution in the second compartment, the waste solution comprising the other components from the effluent.

82-145. (canceled)

146. The kit of claim 75, wherein the outlet of the effluent separation module is configured to be fluidly connected to a container via a tenth tube, and wherein the container is configured to be fluidly connected to the collection reservoir via an eleventh tube.

147-166. (canceled)

167. The kit of claim 1, wherein the kit further comprises:
a first pressure sensor configured to be fluidly connected to the second tube between the first pump and the collection canister, wherein the first pressure sensor is configured to detect the pressure between the first pump and the collection canister;
a second pressure sensor configured to be fluidly connected to the second tube between the first pump and the adipose separation module, wherein the second pressure sensor is configured to detect the pressure between the first pump and the adipose separation module;
a third pressure sensor configured to be fluidly connected to the fourth tube between the collection reservoir and the second pump, wherein the third pressure sensor is configured to detect the pressure between the collection reservoir and the second pump;
a fourth pressure sensor configured to be fluidly connected to the fourth tube between the second pump and the injector, wherein the fourth pressure sensor is configured to detect the pressure between the second pump and the injector;
or a combination thereof.

168-174. (canceled)

175. The kit of claim 1, wherein the kit further comprises:
a first volume sensor configured to be connected to the collection canister and to detect the volume of the mixture within the collection canister;
a second volume sensor configured to be connected to the collection reservoir and to detect the volume of the concentrated adipose tissue within the collection reservoir;
or a combination thereof.

176. (canceled)

177. (canceled)

178. (canceled)

179. The kit of claim 1, wherein the kit further comprises the injector, wherein the injector is configured to receive the concentrated adipose tissue from the collection reservoir via the fourth tube and inject the concentrated adipose tissue into the second anatomical region.

180. (canceled)

181. (canceled)

182. The kit of claim 1, wherein the injector is configured to be fluidly connected to a fifth pressure sensor, wherein the fifth pressure sensor is configured to detect the pressure at which the injector injects the concentrated adipose tissue into the second anatomical region.

183-203. (canceled)

204. A method of use of the kit of claim 1, the method comprising transplanting adipose tissue from the first anatomical region to the second anatomical region using the kit.

205. (canceled)

206. (canceled)

207. A controller for a closed-loop adipose transplant system, wherein
the controller is configured to be communicatively coupled to one or more components of the kit of claim 1, the controller comprising:
a user interface comprising a display showing real-time operating parameters and a control selection panel;
wherein the real-time operating parameters comprise: the volume detected by the first volume sensor, the pressure detected by the first pressure sensor, the pressure detected by the second pressure sensor, the pressure detected by the third pressure sensor, the pressure detected by the fourth pressure sensor, the volume detected by the second volume sensor, the pressure detected by the fifth pressure sensor, or a combination thereof;
wherein the control selection panel displays control parameters and includes: a selector for starting and stopping an adipose transplant procedure upon selection by a user; and one or more selectors for allowing the user to modify one or more of the control parameters;
wherein the control parameters comprise: a minimum volume for the first volume sensor, a maximum volume for the first volume sensor, a minimum pressure for the first pressure sensor, a maximum pressure for the first pressure sensor, a minimum pressure for the second pressure sensor, a maximum pressure for the second pressure sensor, a minimum pressure for the third pressure sensor, a maximum pressure for the third pressure sensor, a minimum pressure for the fourth pressure sensor, a maximum pressure for the fourth pressure sensor, a minimum volume for the second volume sensor, a maximum volume for the second volume sensor, or a combination thereof.

208-217. (canceled)
